(19) United States
(12) Patent Application Publication
(10) Pub. No.: US 2017/0119814 A1
(43) Pub. Date: May 4, 2017

(54) THERAPEUTIC NANOPARTICLES AND METHODS THEREOF

(71) Applicant: ALBERT EINSTEIN COLLEGE OF MEDICINE, INC., Bronx, NY (US)

(72) Inventors: Joel M. FRIEDMAN, South Orange, NJ (US); Mahantesh S. NAVATI, Bronx, NY (US); Adam J. FRIEDMAN, New York, NY (US); Parimala NACHARAJU, Staten Island, NY (US); Aimee KRAUSZ, Bronx, NY (US)

(73) Assignee: Albert Einstein College of Medicine, Inc., Bronx, NY (US)

(21) Appl. No.: 15/318,947

(22) PCT Filed: Jun. 11, 2015
(86) PCT No.: PCT/US2015/035299

§ 371 Date: Dec. 14, 2016

Related U.S. Application Data
(60) Provisional application No. 62/013,259, filed on Jun. 17, 2014.
(60) Provisional application No. 62/032,850, filed on Aug. 4, 2014.
(60) Provisional application No. 62/036,886, filed on Aug. 13, 2014.
(60) Provisional application No. 62/059,226, filed on Oct. 3, 2014.
(60) Provisional application No. 62/074,382, filed on Nov. 3, 2014.

(51) Int. Cl. A61K 33/00; A61K 9/19; A61K 49/00; A61K 41/00; A61K 31/20; A61K 9/16; A61J 3/02; A61K 31/401; A61K 31/198; A61K 31/12; A61K 9/06; A61K 9/00

(52) U.S. Cl. CPC A61K 33/00; A61K 9/06; A61K 9/19; A61K 49/0043; A61K 41/00; A61K 47/4893; A61K 9/0009; A61K 9/1617; A61K 9/1641; A61K 9/1694; A61J 3/02; A61K 9/1652; A61K 31/401; A61K 31/198; A61K 31/12; A61K 41/0057; A61K 31/20

(57) ABSTRACT

Described herein is a method of preparing a hybrid hydrogel paramagnetic nanoparticle. In certain embodiments, the hybrid hydrogel paramagnetic nanoparticle comprises a therapeutic agent. In certain embodiments, the nanoparticle contains alcohol. In certain embodiments, the nanoparticles incorporate fatty acids. Also described herein, is a method of preparing a hybrid hydrogel NO-releasing nanoparticle. In another embodiment, provided herein is a method of preparing a S-nitrosocaptopril hydrogel nanoparticle. Also described herein is a method of preparing a curcumin-based hydrogel nanoparticle. Further, described herein is a method for treating a bacterial infection in a burn wound using curcumin-based hydrogel nanoparticles. Also provided herein is a method of treating a fungal infection using photoactivated curcumin-based hydrogel nanoparticles. In certain embodiments, the fungal infection is caused by dermatophytic fungi.

### RELATED APPLICATIONS

This application claims the benefit of U.S. provisional application serial number U.S. provisional application Ser. No. 62/013,259, filed Jun. 17, 2014, U.S. provisional application Ser. No. 62/032,850, filed Aug. 4, 2014, U.S. provisional application Ser. No. 62/036,886, filed Aug. 13, 2014, U.S. provisional application Ser. No. 62/059,226, filed Oct. 3, 2014, and U.S. provisional application Ser. No. 62/074,382, filed Nov. 3, 2014, which are hereby incorporated by reference in their entireties.

### 1. INTRODUCTION

Disclosed herein is a platform for the preparation of hybrid-hydrogel based nanoparticles that can be: i) loaded with drugs (e.g., chemotherapeutics), nutraceuticals (e.g. curcumin), nitric oxide (NO), nitric oxide precursors, nitrosothiols, imaging agents (e.g., MRI, CT, PET, fluorescence), melanin, plasmids, siRNA, nitro fatty acids, salts and ions (metal and rare earth); and ii) coated with polyethylene glycol (PEG) including derivatized PEG and/or cell/tissue targeting molecules. In certain embodiments, the hybrid-hydrogel nanoparticles are paramagnetic.

Also disclosed herein is a method of enhancing delivery of therapeutic agents in nanoparticles via the use of fatty acids.

Also disclosed herein is a platform for the preparation of nitric oxide (NO) releasing nanoparticles. In certain embodiments, the NO-releasing nanoparticles can be loaded with NO-responsive fluorophores (e.g., diamino fluorescein [DAF]). In certain embodiments, the nanoparticles can be hybrid hydrogel-based nanoparticles. In certain embodiments, the nanoparticles can be paramagnetic nanoparticles. In certain embodiments, the nanoparticles can incorporate an angiotensin converting enzyme (ACE) inhibitor (e.g., captopril).

Also disclosed herein is a platform for the preparation of curcumin-encapsulated nanoparticles. In certain embodiments, the curcumin-encapsulated nanoparticles are hydrogel-based nanoparticles.

Also disclosed herein are methods of treatment with the aforementioned nanoparticles.

### 2. BACKGROUND

Targeted drug delivery is a high priority medical objective. Many drugs are highly effective with respect to “treating” the pathological site (e.g., tumors) but the dosing necessary to achieve efficacy often results in systemic effects that negatively impact the patient to a degree that can range from moderate discomfort to life threatening. A large percentage of drugs fail clinical development due to their inability to be delivered to the disease site at the proper concentration, or because of severe toxic side effects. For example, the majority of individuals with cancer are treated with non-specific chemotherapeutics which have nasty side effects, as they kill not only cancer cells but healthy normal cells as well. A drug delivery mechanism which could specifically transport a therapeutic at high concentration to only cancerous cells while avoiding healthy cells would not only increase the effectiveness of older chemotherapeutics, but could potentially rescue countless drug compounds currently in development and be integrated into new drug designs.

A general approach that allows for delivery of therapeutically effective drug dosing exclusively to the diseased tissue would accomplish two important goals: i) increase the amount of drug delivered to the targeted site while reducing the amount of administered drug; and ii) minimize toxic systemic consequence. Tissue targeting with respect to imaging is another important objective in that the ability to target contrast agents to a specific site allows for an enhancement of diagnostic capability. The combination of contrast and drug delivery (theranostic) in a platform that allows for targeting would provide a synergistic enhanced diagnostic and treatment capability.

Presently, there are three major approaches for targeting the pathological site. The first is the attachment of targeting molecules to either a drug/therapeutic or a drug-loaded nanoparticle. This approach has met with some success but is limited largely due to two factors: 1) the requirement that the drug or nanoparticle remain circulating for sufficient time to allow for accumulation in the target site; and 2) the loss of targeting capability especially for the nanoparticles because of a progressive buildup of adherent plasma proteins on the surface of the nanoparticle that inhibit site recognition by the targeting molecule.

The second major approach is the use of PEGylation. Many disease tissues including many types of tumors have inflamed vasculature that results in “leaky” blood vessels at those sites. Nanoparticles circulating in the blood stream can become trapped at the site of leaky vessels, which can allow for more targeted drug delivery. PEGylation of nanoparticles greatly enhances the probability of the nanoparticles getting trapped in tissues with leaky vessels. PEGylation of nanoparticles has also been shown to enhance crossing of the blood brain barrier. Despite the advantages of PEGylation, there still is a long time window during which the PEGylated nanoparticles must continue to circulate in order to build up enough of a trapped population to achieve therapeutic levels of drug delivery.

The third major approach is the use of coated paramagnetic nanoparticles. This approach uses an external magnet to rapidly localize IV infused paramagnetic nanoparticles (PMNPs) at the target site, thus overcoming the issue of extended circulation times and loss of targeting capability due to progressive buildup of plasma proteins on the surface. For instance, the localized PMNPs can become trapped an extended time at the target site when the target site contains tissues manifesting leaky vasculature as occurs in many tumor and inflamed tissues. These PMNPs are comprised of a solid paramagnetic core (can be iron oxide or gadolinium oxide based) that are coated in order to load a deliverable. The requirement for having to coat the paramagnetic core in order to provide the deliverable, however, limits the applicability of this promising method to molecules that can be loaded onto the surface layer of the PMNP.

As such, there is a need for approaches to targeted drug delivery that increase the amount of drug delivered to the targeted site without increase the amount of administered drug, as well as minimize the systemic toxicity of the drug delivered.

Another high medical objective is the discovery of novel antimicrobial therapies. One such potential antimicrobial therapy is nitric oxide. Nitric oxide (NO), a diatomic gaseous molecule, has an exceedingly short half-life but it has diverse, powerful roles in vivo. Of relevance, it is an essential agent of the innate immune system and is generated and released by macrophages, neutrophils, eosinophils, fibroblasts, epithelial cells, endothelial cells, and glial cells as a method of killing or inhibiting the replication of bacteria, fungi, parasites and viruses. NO exerts antimicrobial activity via reactivity with superoxide anion (forming cytotoxic peroxynitrite), S-nitrosylation of thiol residues in proteins (conformational change), inactivation of enzymes by disruption of iron centers (ribonucleotide reductase, aconitase, ubiquinone reductase), DNA damage, and peroxidation of membrane lipids. NO may also exert indirect antimicrobial effects by upregulating IFNγ, as well as superoxide and hydrogen peroxide release by neutrophils, and its hydrophobic nature allows it to readily traverse cell membranes. In the context of skin and soft tissue infections (SSTIs), NO's vasodilating properties enable necessary components of the immune system to reach the site of infection, further aiding the overall effort to eradicate the invading organism. Thus, with the application of molecules such as NO, which exert antimicrobial effects by a variety of mechanisms, it is unlikely that microbes will develop resistance, as multiple simultaneous gene mutations would be required to develop in the same microbial cell.

Due to the great potential of a multi-mechanistic antimicrobial, a considerable effort has been undertaken to harness NO as a therapeutic. In vivo, NO can be donated from NO-containing molecules and proteins such as S-nitrosoglutathione (GSNO), S-nitrosoalbumin, S-nitrosylated hemoglobin, and even iron nitrosyl hemoglobin via transnitrosylation. Inspired by transnitrosylation in vivo, a variety of S-nitrosothiol (RSNO) therapeutics have emerged (i.e., S-nitroso-N-acetylcysteine, S-nitroso-N-acetyl-penicillamine), which exert effects by transferring NO from one thiol group to another. RSNO therapeutics exhibit similar activity to NO by acting as long-lasting vasodilators (without drug tolerance), preventing platelet aggregation, and exhibiting antimicrobial effects.

Sustained generation of GSNO from a nitric oxide releasing nanoparticle platform (NO-np) in combination with solubilized glutathione (GSH) has been shown to be highly effective against bacterial species in vivo (Pseudomonas aeruginosa) and in vitro (methicillin Resistant Staphylococcus aureus (MRSA), Escherichia coli, P. aeruginosa, and Klebsiella pneumoniae). Interestingly, when exposed to an aliquot of GSNO at the same concentration generated from the nanoparticles, no antibacterial activity was observed. Thus, it is likely that sustained levels of GSNO generated by the nanoparticle platform are necessary for bactericidal activity.

Thus, while the combination of NO-np and GSH was found to be effective both in vitro and in vivo, the practical utility of this combination is negated by the instability of GSH in light and ambient temperature, as well as the requirement of this combination to be in suspension, which will ultimately exhaust generated GSNO over time. Therefore, there is a need for a platform that itself can both release NO and facilitate transnitrosylation.

Another foremost medical objective is the discovery of novel treatment regimens for traumatic injuries, such as burns. Among traumatic injuries, burns represent a significant source of morbidity and mortality. The avascular wound bed provides an ideal environment for microbial growth, facilitating penetration of pathogens into underlying tissue, with potential for hematogenous dissemination. Up to 75% of deaths following burn injury relate to infection, most commonly caused by methicillin-resistant Staphylococcus aureus (MRSA) and Pseudomonas aeruginosa. Currently employed antimicrobial agents possess limited utility due to toxicity, incomplete antimicrobial coverage, inadequate wound bed penetration, and growing bacterial resistance. In addition, mainline treatments such as silver sulfadiazine may delay burn wound healing. As such, there is a need for a new strategy for treating infections following burn injuries.

Finally, another important medical objective encompasses finding new treatments for fungal infections. For example, dermatophytic fungi utilize nutrients from keratinized tissue, such as skin, hair and nails, and the incidence of dermatophytic fungal infections has increased due to the growing number of immunocompromised individuals and rising antimicrobial resistance rates. Fungal resistance has been particularly pronounced for Trichopyton rubrum, the most common organism implicated in cutaneous fungal infections, and the cause of invasive infections like Majocci's granuloma as well as onychomycosis. Currently utilized therapeutics effectively target metabolically active organisms but do not eliminate the dormant spores, leading to treatment failure despite systemic therapy. As such, there is a need for a new strategy for treating fungal infections.

### 3. SUMMARY

Described herein is a method of preparing a hybrid hydrogel paramagnetic nanoparticle. In one aspect, the method comprises the steps of: (a) hydrolyzing tetramethyl orthosilicate (TMOS); (b) sonicating the hydrolyzed TMOS to form a TMOS solution; (c) mixing deionized water with gadolinium chloride hexahydrate, europium chloride hexahydrate, PEG, chitosan, and methanol to form a mixture; (d) vortexing the mixture; (e) mixing the TMOS solution, an amine-containing silane, and ammonium hydroxide with the mixture to form a hydrogel mixture; (f) vortexing the hydrogel mixture to form a hydrogel; (g) lyophilizing the resulting hydrogel to form a dry material; (h) ball-milling the dry material to form a powder; and (i) mixing the resulting powder with an amine-binding PEG. In certain embodiment, the amine-containing silane is 3-aminopropylmethoxysilane. In one or more embodiments, the hybrid hydrogel paramagnetic nanoparticle comprises a therapeutic agent, such as a chemotherapeutic, a nutraceutical, nitric oxide, a nitrosothiol, an imaging agent, melanin, a plasmid, siRNA, a nitro fatty acid, salts and ions or a combination thereof.

Also described herein, in at least one embodiment, is a method of preparing a hybrid hydrogel NO-releasing nanoparticle comprising the steps of: (a) hydrolyzing TMOS; (b) sonicating the hydrolyzed TMOS to form a TMOS solution; (c) mixing an unsaturated fatty acid, with sodium nitrite, a buffer solution, PEG, chitosan, and methanol to form a mixture; (d) vortexing the mixture; (e) mixing the TMOS solution and an amine-containing silane with the mixture to form a hydrogel mixture; (f) vortexing the hydrogel mixture to form a hydrogel; (g) lyophilizing the resulting hydrogel to form a dry material; and (h) ball-milling the dry material to form a powder. In certain embodiments, the unsaturated fatty acid is a oleic acid, linoleic acid, or conjugated linoleic acid.

In another embodiment, provided herein is a method of preparing a S-nitrosocaptopril hydrogel nanoparticle comprising the steps of: (a) hydrolyzing TMOS to form a mixture; (b) sonicating the mixture; (c) mixing the sonicated mixture with a buffer mixture, PEG, and phosphate containing nitrite and captopril to form a hydrogel; (d) lyophilizing the resulting hydrogel to form a dry material; and (e) ball-milling the dry material to form a powder. Further, provided herein is a composition comprising the S-nitrosocaptopril hydrogel nanoparticles, wherein the concentration of the nanoparticles in the composition is 1-10 mg/mL.

In one embodiment, provided herein is a method of treating a bacterial infection, comprising at least the step of administering to patient a therapeutically effective amount of a composition comprising the S-nitrosocaptopril hydrogel nanoparticles. In certain embodiments, the bacterial infection is caused by E. coli. In at least one embodiment, the bacterial infection is caused by MRSA.

Also described herein, in at least one embodiment, is a method of preparing a curcumin-based hydrogel nanoparticle comprising the steps of: (a) hydrolyzing TMOS to form a mixture; (b) sonicating the mixture on ice; (c) mixing a buffer solution, PEG, and curcumin dissolved in methanol to form a mixture; (d) vortexing the mixture; (e) mixing the TMOS solution with the mixture to form a hydrogel mixture; (f) vortexing the hydrogel mixture to form a hydrogel; (g) lyophilizing the resulting hydrogel to form a dry material; and (h) ball-milling the dry material to form a powder. Also provided herein is a method of treating a fungal infection, comprising at least the steps of: administering to a patient a therapeutically effective amount of the curcumin-based hydrogel nanoparticles; and photoactivating the curcumin-based hydrogel nanoparticles with a dose of a light source. In at least one embodiment, the light source emits blue light. In certain embodiments, the light is a full spectrum light. In certain embodiments, the blue light is at a wavelength of 400 to 440 nm. In certain embodiments, the blue light is at a wavelength of 408 to 434 nm. In at least one embodiment, the dose of light is 10-15, 15-20, 20-25, 25-30, 30-35, 35-40, 40-50 J/cm². In one or more embodiments, the concentration of curcumin in the nanoparticles is 1.0-1.5, 1.5-2, 2-2.5, 2.5-3, 3-3.5, 3.5-4, 4-4.5, 4.5-5, 5-5.5, 5.5-6, 6-6.5, 6.5-7, 7-7.5, 7.5-8, 8-8.5, 8.5-9, 9-9.5, 9.5-10, 10-20, 20-30, 30-40 μg/mL. In certain embodiments, the fungal infection is caused by a dermatophytic fungus. In certain embodiments, the fungal infection is caused by Trichopyton rubrum.

Also provided herein is a method of treating a bacterial infection in a burn wound, comprising at least the step of administering to a patient a therapeutically effective amount of a curcumin-based hydrogel nanoparticles. In certain embodiments, the bacterial infection is caused by MRSA. In certain embodiments, the bacterial infection is caused by Pseudomonas aeruginosa. Further provided herein, in at least one embodiment, is a method of treating a burn wound, comprising at least the step of administering to a patient a therapeutically effective amount of curcumin-based hydrogel nanoparticles. In certain embodiments, the curcumin-based hydrogel nanoparticles are administered to the wound via coconut oil.

In one or more embodiments, provided herein is a method of reducing blood pressure and controlling inflammation, comprising at least the step of administering to a patient a therapeutically effective amount of a curcumin-based hydrogel nanoparticles. In certain embodiments, the curcumin-based hydrogel nanoparticles are administered to the wound via coconut oil.

### 4. BRIEF DESCRIPTION OF THE FIGURES

FIG. 1A. Structure of nitric oxide-releasing hybrid hydrogel nanoparticles as displayed by a scanning electron microscopy (SEM; bar 100 nm);

FIG. 1B. Graphical representation of the analytical sizing of nitric oxide-releasing hybrid hydrogel nanoparticles performed using dynamic light scattering (DLS).

FIG. 1C. Graphical representation of the release of nitric oxide from the nitric oxide-releasing hybrid hydrogel nanoparticles once placed in an aqueous environment over the course of 8 hours.

FIG. 2. Size characterization of S-nitrosocaptopril nanoparticles (SNO-CAP-np). (A) Graphical representation of SNO-CAP-np diameter, measured via dynamic light scattering (DLS). The average diameter weighted by intensity was 377.8±16.4 nm, and the curve represents 40 acquisition attempts. Since SNO-CAP-np swell with moisture, the diameter is likely an overestimate of dry size. (B) SNO-CAP-np were visualized via scanning electron microscopy (accelerating voltage 3 kV).

FIG. 3. Graphical representation of NO release from SNO-CAP-np in PBS (1 mg/mL), evaluated over 12 hours via chemiluminescent NO analyzer (Sievers NO analyzer, Model 280i).

FIG. 4. Graphical representation of GSNO formation reaction. (A) Revere-Phase High Performance Liquid Chromatography (RPHPLC) analysis of the SNO-CAP-np+GSH reaction. Twenty mg/mL SNO-CAP-np with 20 mM GSH was incubated at room temperature, as was a control suspension of SNO-CAP-np. Their respective chromatograms represent aliquots taken after one minute and diluted 50×. GSH and GSNO standards were analyzed by RPHPLC at 0.1 mM. Peaks 1 and 2 in the SNO-CAP-np+GSH reaction were identified as GSH and GSNO, respectively. (B) Time course of GSNO formation. GSNO peak area was evaluated for SNO-CAP-np (20 mg/mL)+GSH (20 mM) reaction mixture at various time points and compared to the GSNO standard to determine real quantities of GSNO formation over time.

FIG. 5. Graphical representation of E. coli and MRSA susceptibility to SNO-CAP-np. (A) E. coli with SNO-CAP-np (B) MRSA with SNO-CAP-np (C) E. coli with captopril (D) MRSA with captopril. Error bars represent SEM.

FIG. 6. Graphical representation of CFU assay. (A) E. coli with SNO-CAP-np (B) MRSA with SNO-CAP-np (C) E. coli with captopril (D) MRSA with captopril. After E. coli and MRSA were incubated at 37° C. for 24 h with either SNO-CAP-np or captopril in TSB (one colony/mL diluted 200-fold), 10 μL was aspirated and further diluted 100-fold in PBS. The dilutions were plated in 100 μL aliquots on TSA, and colony forming units (CFU's) were quantified following 24 h incubation at 37° C. The highest concentration of SNO-CAP-np (10 mg/mL) contained 2.76 mM captopril. Symbols denote p-value significance compared to untreated controls (*P=0.0007, **P<0.0001, †P=0.02, ††P=0.0003, #P=0.026) as calculated by unpaired t-test analysis.

FIG. 7. SNO-CAP-np are non-toxic in vivo. (A) Graphical representation of percent mortality as a function of exposure concentration and treatment material (N=24). (B) Zebrafish embryos (120 hpf) exposed to 250 ppm of nanomaterial. (i) Untreated, (ii) Control-np, (iii) Alexa 568-np, and (iv) SNO-CAP-np. Photographs demonstrate the absence of all malformations in zebrafish exposed to control-np, Alexa 568-np, or SNO-CAP-np as indicated by reference to unexposed control zebrafish.

FIG. 8. Optimization of aPI conditions. (A) Graphical representation of the effect of varying the PS concentration on fungal growth, as determined by colony forming units (CFU), using a constant light source of 40 J/cm2. (B) Graphical representation of the effect of varying the light dose using a constant PS concentration of 10 μg/mL. Untreated T. rubrum (C), Blue light alone (B.L.) and PS without photoactivation were used as controls. ***Compared to untreated, blue light and PS without photoactivation and compared to lowest PS concentration of same group. †Compared to untreated control. ***p<0.0001; †p<0.05. Data are a composite of three independent experiments with each treatment group performed in triplicate. The results are expressed as the mean±SEM.

FIG. 9. Fungal growth curves after incubation with ground-state and photoactivated curcumin (A-B) Incubation of T. rubrum with a range of (A) curcumin (curc) and (B) curc-np concentrations in the ground-state. (C) Fungal growth after aPI using a PS concentration of 10 μg/mL. Each treatment per group was performed in triplicate and data are a composite of two independent experiments. The results are expressed as the mean±SEM.

FIG. 10. Evaluation of ROS and RNS production after aPI. Detection of ROS levels using H2DCFDA probe, expressed as a (A) representative histogram and (D) cumulative bar plot. Detection of NO. levels using DAF-FM probe, expressed as a (B) representative histogram and (E) cumulative bar plot. Detection of ONOO. levels using DHR 123 probe, expressed as a (C) representative histogram and (F) cumulative bar plot. Dark toxicity controls did not differ significantly from untreated T. rubrum (data not represented). ***Compared to untreated control. ###Compared to cure group. MFI. Mean fluorescence intensity. ***,###p<0.0001. Each treatment per group was performed in triplicate and are a composite of two independent experiments. The results are expressed as the mean±SEM.

FIG. 11. Evaluation of aPI mechanism of action. (A and B) Graphical representation of photodynamic inhibition performed in the presence of ROS and RNS scavengers, with degree of fungal growth evaluated by colony forming unit (CFU) quantification. (A) Treatment with ONOO. scavenger (FeTPPs). (B) Treatment with NO. scavenger (Carboxy-PTIO). (C) Graphical representation of apoptosis assay performed after aPI therapy. ***Compared to aPI treatment in the absence of incubation with scavengers. *Compared to untreated T. rubrum control. *p<0.05, ***p<0.0001. Each treatment per group was performed in triplicate and data is a composite of two independent experiments. The results are expressed as mean±SEM.

FIG. 12. Graphical representations of phagocytosis assay and in vivo study. (a) CFU quantification of macrophages challenged with T. rubrum cells and treated with aPI therapy. (b) BALB/c mice treated with aPDT. # Compared to untreated control (UTC), dark toxicity and blue light 10 J/cm2 (B.L.) controls. *,**Compared to all other groups. B.L. Blue light 10 J/cm2 (17 minutes). *,#p<0.05. **p<0.01. Each treatment per group was performed in triplicate and data is a composite of two independent experiments. The results are expressed as the mean±SEM.

FIG. 13. Clinical site of T. rubrum infection, Majocci's granuloma.

FIG. 14. Characterization and toxicity of curcumin-encapsulated nanoparticles (curc-np). (A) Scanning electron microscopy revealed distinct spherical nanoparticles (left bar=200 nm, right bar=100 nm). (B) Graphical representation of monomodal size distribution quantified by dynamic light scattering indicated a narrow size range with average diameter 222±14 nm (C) Graphical representation of release %, which occurred in a controlled and sustained fashion, reaching 81.5% after 24 hours. (D) Graphical representation of percent mortality at 120 hours post-fertilization (hpf) as a function of exposure concentration. Mortality was not significant for embryos exposed to curc-np in comparison to fish water control. (E) Representative images of zebrafish embryos at 120 hpf: control (top) and exposed to curc-np (bottom). No significant differences were observed in larval morphology or behavioral endpoints (p≦0.05 for each endpoint evaluated, Fisher's Exact test). Error bars denote SEM.

FIG. 15. Curc-np inhibit planktonic growth of Gram-positive and -negative organisms. Representative 24-hour growth curves demonstrate susceptibility of (A) MRSA isolates (n=8) and (B) Pseudomonas aeruginosa isolates (n=4) to 5 mg/ml of curc-np and control np (np). Time points average results for 3 measurements. Statistical analysis conducted using 2-way ANOVA. Error bars denote SEM.

FIG. 16. Curc-np induce cellular damage of MRSA. High-power transmission electron microscopy demonstrated interaction of nanoparticles (arrows) with MRSA cells. (A) Untreated MRSA showed uniform cytoplasmic density and central cross wall surrounding a highly contrasting splitting system. (B) After 24 hours, cells incubated with control np 5 mg/ml did not exhibit any changes in cellular morphology as compared to the untreated control. (C) After 6 hours, cells incubated with curc-np 5 mg/ml exhibited distortion of cellular architecture and edema, followed by lysis and extrusion of cytoplasmic contents after 24 hours (D). Error bars denote SEM. All scale bars=500 nm.

FIG. 17. Curc-np decrease bacterial burden of full-thickness burns. Graphical representation of wound bacterial burden (CFU; colony forming unit) in mice infected intradermally with 5×10⁸ MRSA cells was determined by amount of CFU growth (n=10 wounds per group). On day 3 (A) and day 7 (B) after infection, bacterial burden of curc-np-treated wounds was significantly lower than untreated, coconut oil (CO)-treated, and control np (np)-treated wounds (***p≦0.001, Student's t-test). Error bars denote SEM.

FIG. 18. Curc-np accelerate wound healing in a murine burn model. (A) Graphical representation of wound size analysis (relative area versus initial area), which revealed statistically significant acceleration of wound healing in mice treated with curc-np as compared to untreated, coconut oil control (CO), silver sulfadiazine (SS), and control np (np; p≦0.0001, 2-way ANOVA). Time points are the averages of the results for 10 measurements, and error bars denote SEM. (B) Representative images of wound healing from days 2-14. Topical administration with curc-np decreased eschar size and qualitatively accelerated healing compared to all other groups. CO (vehicle) control did not differ significantly from untreated control (data not shown). Error bars denote SEM. Scale bar=5 mm.

FIG. 19. Curc-np enhance formation of granulation tissue, collagen deposition and neoangiogenesis. (A) Histologic analysis of wound tissue from day 13 using hematoxylin and eosin (H&E) and Masson's trichrome staining. On H&E (magnification 4×, bar=500 um; 10×, bar=100 um), untreated control, silver sulfadiazine (SS), and control np (np)-treated wounds exhibited fibrinous debris and inflammatory granulation tissue compared to the accelerated maturation of curc-np-treated wounds. On trichrome (magnification 40×, bar=100 um), increased collagen deposition, more orderly orientation of fibers, and decreased necrosis were appreciated in curc-np-treated wounds compared to all other groups. (B) Graphical representation of quantitative measurement of collagen intensity in 10 representative fields of the same size (in arbitrary units, A.U.). (C) Graphical representation of quantitative measurement of microvessels based on CD34 staining of excised tissue in 10 representative fields of the same size (magnification 40×). ***p≦0.0001, Student's t-test. Error bars denote SEM.

FIG. 20. Nano-curcumin-treated mice exhibited a lower OA histologic score (using the OARSI scoring system) compared to OA mice treated with vehicle. *p<0.05. n=3/group.

FIG. 21. Safranin O staining of OA mice cartilage treated with nano-encapsulated curcumin compared with vehicle treatment alone (coconut oil).

FIG. 22. Distance traveled by nano-curcumin-treated mice in an open box assay, compared with vehicle-treated and treatment-naïve mice. *p<0.05, n=3/group.

FIG. 23. Frequency of rearing (standing on hind limbs) by nano-curcumin-treated mice compared with vehicle-treated and treatment-naïve mice in an open box assay. *p<0.05, n=3/group.

FIG. 24. Blood pressure (mean artery pressure [MAP]) over time. Effect of treating hamsters with NO-nanoparticles with myristic acid compared with nanoparticles without myristic acid and untreated. Groups: 1) NO-nanoparticles with myristic acid (n=3) [NO-np-C14H28O2]; 2) NO-no without myristic acid (n=3) [NO-np]; and 3) untreated (n=5).

FIG. 25. Heart rate (beats per minute [bpm])) over time. Effect of treating hamsters with NO-nanoparticles with myristic acid compared with nanoparticles without myristic acid and untreated. Groups: 1) NO-nanoparticles with myristic acid (n=3) [NO-np-C14H28O2]; 2) NO-no without myristic acid (n=3) [NO-np]; and 3) untreated (n=5).

FIG. 26. Levels of NO-related products (S-nitrothiols [A], nitrite [B], and nitrate [C]) in the bloodstream following treatment. Groups: 1) NO-nanoparticles with myristic acid (n=3) [NO-np-C14H28O2]; 2) No-no without myristic acid (n=3) [NO-np]; and 3) untreated (n=5).

### 4.1 DEFINITIONS

When referring to the compounds and methods provided herein, the following terms have the following meanings unless otherwise indicated.

As used herein, the term “agent” refers to any molecule, compound, and/or substance for use in the prevention, treatment, management and/or diagnosis of a disease, including but not limited to cancer.

As used herein, the term “amount,” as used in the context of the amount of a particular cell population or cells, refers to the frequency, quantity, percentage, relative amount, or number of the particular cell population or cells.

As used herein, the term “bind” or “bind(s)” refers to any interaction, whether direct or indirect, that affects the specified receptor (target) or receptor (target) subunit.

As used herein, the term “cancer” refers to a neoplasm or tumor resulting from abnormal uncontrolled growth of cells. The term “cancer” encompasses a disease involving both pre-malignant and malignant cancer cells. In some embodiments, cancer refers to a localized overgrowth of cells that has not spread to other parts of a subject, i.e., a benign tumor. In other embodiments, cancer refers to a malignant tumor, which has invaded and destroyed neighboring body structures and spread to distant sites. In yet other embodiments, the cancer is associated with a specific cancer antigen.

As used herein, the term “cancer cells” refers to cells that acquire a characteristic set of functional capabilities during their development, including the ability to evade apoptosis, self-sufficiency in growth signals, insensitivity to anti-growth signals, tissue invasion/metastasis, significant growth potential, and/or sustained angiogenesis. The term “cancer cell” is meant to encompass both pre-malignant and malignant cancer cells.

As used herein, the term “cytotoxic” or the phrase “cytotoxicity” refers to the quality in a compound of causing adverse effects on cell growth or viability. The “adverse effects” included in this definition are cell death and impairment of cells with respect to growth, longevity, or proliferative activity.

As used herein, the terms “disorder” and “disease” are used interchangeably to refer to a pathological condition in a subject.

As used herein, the term “effective amount” refers to the amount of a therapy that is sufficient to result in the prevention of the development, recurrence, or onset of a disease and one or more symptoms thereof, to enhance or improve the prophylactic effect(s) of another therapy, reduce the severity, the duration of a disease, ameliorate one or more symptoms of a disease, prevent the advancement of a disease, cause regression of a disease, and/or enhance or improve the therapeutic effect(s) of another therapy.

As used herein, the phrase “elderly human” refers to a human 65 years old or older, preferably 70 years old or older.

As used herein, the phrase “human adult” refers to a human 18 years of age or older.

As used herein, the phrase “human child” refers to a human between 24 months of age and 18 years of age.

As used herein, the phrase “human infant” refers to a human less than 24 months of age, preferably less than 12 months of age, less than 6 months of age, less than 3 months of age, less than 2 months of age, or less than 1 month of age.

As used herein, the term “in combination” in the context of the administration of a therapy to a subject refers to the use of more than one therapy (e.g., prophylactic and/or therapeutic). The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

As used herein, the terms “manage,” “managing,” and “management” in the context of the administration of a therapy to a subject refer to the beneficial effects that a subject derives from a therapy (e.g., a prophylactic or therapeutic agent) or a combination of therapies, while not resulting in a cure of cancer. In certain embodiments, a subject is administered one or more therapies (e.g., one or more prophylactic or therapeutic agents) to “manage” cancer so as to prevent the progression or worsening of the condition.

As used herein, the phrase “pharmaceutically acceptable” means approved by a regulatory agency of the federal or a state government, or listed in the United States Pharmacopeia, European Pharmacopeia, or other generally recognized pharmacopeia for use in animals, and more particularly, in humans.

In certain embodiments, the compositions comprising the modified nanoparticles are administered to a patient, preferably a human, as a preventative measure against such diseases. As used herein, “prevention” or “preventing” refers to a reduction of the risk of acquiring a given disease or disorder. In a preferred mode of the embodiment, the compositions comprising the modified nanoparticles are administered as a preventative measure to a patient, preferably a human, having a genetic predisposition to the above identified conditions. In another preferred mode of the embodiment, the compositions comprising the modified nanoparticles are administered as a preventative measure to a patient having a non-genetic predisposition to the above-identified conditions.

As used herein, the terms “purified” and “isolated” when used in the context of a compound or agent (including proteinaceous agents such as antibodies) that can be obtained from a natural source, e.g., cells, refers to a compound or agent that is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells.

As used herein, the phrase “small molecule(s)” and analogous terms include, but are not limited to, peptides, peptidomimetics, amino acids, amino acid analogs, polynucleotides, polynucleotide analogs, nucleotides, nucleotide analogs, and other organic and inorganic compounds (i.e., including hetero-organic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, organic or inorganic compounds having a molecular weight less than about 100 grams per mole, and salts, esters, and other pharmaceutically acceptable forms of such compounds.

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

In at least one embodiment, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

Concentrations, amounts, cell counts, percentages, and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 5. DETAILED DESCRIPTION

In the following detailed description, numerous specific details are set forth to provide a thorough understanding of claimed subject matter. However, it will be understood by those skilled in the art that claimed subject matter may be practiced without these specific details. In other instances, methods, apparatuses, or systems that would be known by one of ordinary skill in the art have not been described in detail so as not to obscure claimed subject matter. It is to be understood that particular features, structures, or characteristics described may be combined in various ways in one or more implementations.

In general, the present application relates to the preparation and administration of modified nanoparticles and/or pharmaceutical compositions comprising modified nanoparticles. In one or more embodiments, methods of preparing modified nanoparticles and/or pharmaceutical compositions comprising modified nanoparticles are provided. In one or more embodiments, methods of treating or preventing or managing a disease or disorder in humans by administering a pharmaceutical composition comprising an amount of modified nanoparticles are provided. Also provided herein is a method of treatment comprising administering to the subject an effective amount of one or more of the nanoparticles disclosed herein and a pharmaceutically acceptable carrier. Further, provided herein is a pharmaceutical composition comprising any of the nanoparticles disclosed herein and a pharmaceutically acceptable carrier.

In certain embodiments, the modified nanoparticles comprises 10-20, 20-30, 30-40, 40-50, 50-60, 60-70, 70-80, 80-90, 90-100 μg of therapeutic agent per mg of nanoparticle. In certain embodiments, the modified nanoparticles comprise 22-44, 24-40, 50-60 μg of therapeutic agent per mg of nanoparticle.

In certain embodiments, the modified nanoparticles comprise 10-20, 20-30, 30-40, 40-50, 50-60, 60-70, 70-80, 80-90, 90-100 μg of therapeutic agent per mg of nanoparticle per unit time. In certain embodiments, the modified nanoparticles comprises 22-44, 24-40, 50-60 μg of therapeutic agent per mg of nanoparticle per unit time. In certain embodiment, the unit time is 1-5, 5-10, 10-15, 15-20, 20-25, 25-30, 30-35, 35-40, 40-45, 45-50, 50-60 secs, 1-2 mins, 2-5 mins, 5-10 mins, 10-30 mins, 30-60 mins.

In certain embodiments, the modified nanoparticles have a core size of 50-60, 60-70, 70-80, 80-90, 90-100, 100-110, 110-120, 120-130, 130-140, 140-150, 150-160, 160-170, 170-180, 180-190, 190-200, 200-300, 300-400, and 400-500 nm. In certain embodiment, modified nanoparticles have a core size of 70-150 nm.

In certain embodiments, the modified nanoparticles comprises 2, 3, 4, 5, 6, 7, 8, 9, 10, 20, 30, 40, 50 folds more therapeutic agents than nanoparticles that do not have the modification(s) described in the present disclosure.

In certain embodiments, the modified nanoparticles as disclosed herein have improved permeability crossing the blood brain barrier as compared to other nanoparticles having similar size. In certain embodiments, the modified nanoparticles have a nanoparticle core that has similar size as other previously known nanoparticles and yet has an increased permeability crossing the blood brain barrier by the order of at least 10, 10-10², 10²-10³, 10³-10⁴, 10⁴-10⁵. In certain embodiments, the modified nanoparticles are 2, 3, 4, 5, 6, 7, 8, 9, 10, 20, 30, 40, 50 folds more efficient in penetration across the blood brain barrier than nanoparticles that does not have the modification(s) described in the present disclosure.

In certain embodiments, the modified nanoparticles are 2, 3, 4, 5, 6, 7, 8, 9, 10, 20, 30, 40, 50 folds more efficient in entering a cell at the location that the nanoparticles are targeted in a subject than nanoparticles that do not have the modification(s) described in the present disclosure. In certain embodiments, the cells are cancer cells. In certain embodiments, the cells are glioblastoma cells. In certain embodiments, the cells are cardiac cells, blood vessel cells and capillary cells. In certain embodiments, the cells are bone marrow, spleen, brain, bone, etc.

In certain embodiments, the modified nanoparticles have a size dispersion of 0-5%, 5-15%, 15-20%, 20-25% and 25-30%. In certain embodiments, the modified nanoparticles have a size dispersion of less than 1%. In certain embodiments, the modified nanoparticles have a size dispersion of less than 0.1%.

In certain embodiments, the modified nanoparticles of the present application can be formed in sizes having a diameter in dry form, for example, of 10 nm to 1000 μm, preferably 10 nm to 100 μm, or 10 nm to 1 μm, or 10 nm to 500 nm, or 10 nm to 100 nm. Preferably, the nanoparticles have an average diameter of less than 500 nm.

### 5.1 Paramagnetic and Non-Paramagnetic Hybrid-Hydrogel Based Nanoparticles

As described herein, a platform has been developed for the preparation of hybrid-hydrogel based nanoparticles. In certain embodiments, the nanoparticles are paramagnetic. In certain embodiments, the nanoparticles can be loaded with therapeutic agents including, but not limited to: drugs (e.g. chemotherapeutics), nutraceuticals (e.g. curcumin), peptides, thiol-containing small molecules, anti-inflammatories, nitric oxide (NO), NO precursors, nitrosothiols, NACSNO (the S-nitrosothiol derivative of N-acetyl cysteine), imaging agents (MRI, CT, PET, fluorescence), melanin, plasmids, tadalophil, doxorubicin, siRNA, nitro fatty acids, and salts and ions (metal and rare earth). In one or more embodiments, the nanoparticles can be coated with PEG including derivatized PEG and/or cell or tissue targeting molecules. The nanoparticles can be used for both topical and systemic applications. In one or more embodiments, the nanoparticles can form a very fine powder when dry and a uniform suspension when added to liquid solvents (e.g., water, alcohol, DMSO).

For the hybrid-hydrogel based nanoparticles of the present application, the use of the label “hybrid” refers to the combination of a hydrogel with a glass-like interior matrix. Here, glass is used to refer to the amorphous network of hydrogen bonds. This hydrogen bonding network loosens in the presence of water, which initiates the release of the deliverable encapsulated in this matrix.

In certain embodiments, the hybrid-hydrogel based nanoparticles of the present application have the ability to load a wide variety of deliverables into the interior of the nanoparticle with control over release profiles. The nanoparticle platform utilizes a hydrogel technology with additives that created a glass like interior derived from a strong hydrogen bonding network derived from the interaction of chitosan with the side chains of the polymers comprising the hydrogel. This combination provides both a robust nanoparticle framework and an interior that loosen upon exposure to moisture thus allowing for slow sustained release of drugs. The nature of the preparative phase allows for easy loading of virtually any type of biological or therapeutic agent of the appropriate dimensions.

In one or more embodiments, the nanoparticle platform has the flexibility of allowing for tuning of the interior by doping the hydrogel using different trimethoxysilane derivatives added to the tetramethoxy or tetraethoxy silane (Tetramethyl orthosilicate [TMOS] and Tetraethyl orthosilicate [TEOS], respectively) that is used to create the hydrogel network. For example, TMOS or TEOS can be doped with trimethoxysilane derivatives that, at their fourth conjugation site (i.e., Si(OCH3)3(X)), contains derivatives such as a thiol-containing side chain, a lipid-containing side chain, a PEG-containing side chain, or an alkyl side chain of variable length. This doping allows for the introduction of side chains that can modify the over charge of the nanoparticles, tune the hydrophobicity and polarity of the interior, and introduce reactive groups that allow for chemical modifications on the surface (e.g., thiols, amines). This capability allows for control of customize loading and release properties of the nanoparticles to match the deliverable and the therapeutic application.

In one or more embodiments, the nanoparticle platform also allows for the introduction of different size PEGs into the hydrogel matrix. The size of the introduced PEG can be used to control the rate of release of the loaded drugs.

As mentioned above, the nanoparticles of the present application can be paramagnetic. In one or more embodiments, the hybrid hydrogel platform of the nanoparticle is transformed into one that is paramagnetic by the incorporation of gadolinium and/or europium salts into the hydrogel platform. This results in a highly paramagnetic nanoparticle with all the benefits and drug delivery capabilities of a non-paramagnetic hydrogel platform. The paramagnetic capability of the nanoparticle allows for the use of an external magnet to create rapid localization of the nanoparticles at the site of magnet. In at least one embodiment, the resulting paramagnetic nanoparticles can be further modified by attaching PEG (including derivatized PEG) and/or cell-targeting molecules to the surface.

In one or more embodiments, the hydrogel nanoparticle platform allows for the generation and slow release of nitric oxide from within the nanoparticle. This capability allows for slow, sustained release of nitric oxide at the site of the targeted tissues.

In one or more embodiments, the hybrid-hydrogel nanoparticles of the present application are also designed to make the resulting nanoparticles more uniform with respect to size distribution and more compact with respect to the internal polymeric network (resulting in a slower release profile). In at least one embodiment, the nanoparticle platform includes alcohol, which reduces water content (decreases the internal water content) and thus enhance the hydrogen bonding network of the nanoparticles. The use of increased fractions of alcohol in the preparation phase can result in smaller nanoparticles with a narrower distribution of sizes, and slower release profiles. Toxicity due to the use of alcohol is not an issue because of the lyophilization process, which removes all volatile liquids including free water and alcohol.

Further, in one or more embodiments, one or more amine groups can be incorporated into the polymeric network of the nanoparticle through the addition of amine-containing silanes (e.g., aminopropyltrimethoxysilane) with TMOS or TEOS for example, which accelerates the polymerization process and also contributes to a tighter internal hydrogen bonding network. The addition of amine-containing silanes can also contribute to general improvement in the suspension qualities of the nanoparticles. Moreover, the addition of amine groups can help in the attachment of PEGs, peptides, and other amine-binding molecules on the surface of the nanoparticles as a means of extending systemic circulation time and increasing the probability of localization at a target site with leaky vasculature. The net effect of these additions are nanoparticles that release drugs and additives more slowly and more uniform in size distribution. Further, these modifications improve the suspension properties of the nanoparticles (e.g., minimize aggregation), allow for tuning of the average size of the nanoparticles, and allow for delivery of nitro fatty acids and highly lipophilic molecules.

In at least one aspect, the present application provides for a method of enhancing the delivery of therapeutic agents, imaging agents, and theranostics in nanoparticles via the use of fatty acids. In one or more embodiments, the method comprises incorporating fatty acids such as myristic acid, oleic acid, and other conjugated fatty acids (e.g., linoleic acid, conjugated linoleic acid) individually or in combination into the platform for hybrid-hydrogel based nanoparticles. When these are included in the nanoparticle, the resulting nanoparticles can contain nitro fatty acids, which are highly anti-inflammatory and potentially chemotherapeutic. Alternatively, nitro fatty acids can be prepared and then incorporated into the recipe for generating the nanoparticles. The introduction of oleic acid or conjugated linoleic acid, and/or other unsaturated fatty acids into the nanoparticle also provides a lipophilic interior to the nanoparticles that will enhance loading of lipophilic deliverables. The incorporation of one or more fatty acids into the nanoparticle platform can enhance skin penetration, sublingual and suppository-based (e.g., rectal, vaginal) delivery, and systemic delivery via uptake from the gut subsequent to oral ingestion. Specifically, the incorporation of myristic acid into the nanoparticle platform can facilitate improvements in cardiovascular endpoints (e.g., blood pressure, heart rate), and erectile dysfunction. In an alternative embodiment, the one or more fatty acids can be applied to the coatings of gadolinium oxide-based paramagnetic nanoparticles as a means of facilitating systemic delivery via oral, sublingual, or suppository routes.

Another modification to the hybrid-hydrogel nanoparticles include doping the TMOS or TEOS with trimethoxy silane derivates that at their fourth conjugation site (e.g., Si(OCH3)3(X)) contains derivatives such as a thiol-containing side chain, a lipid-containing side chain, a PEG-containing side chain, or an alkyl side chain of variable length. Other additives can also be added to the nanoparticles to enhance its physical properties, such as polyvinyl alcohols.

As mentioned above, in at least one embodiment, the hybrid-hydrogel nanoparticles can be loaded with melanin as a therapeutic agent. This embodiment can be used to demonstrate (via photo-acoustic imaging) magnet-induced localization of the nanoparticles in a tumor with no evidence of systemic toxicity.

As mentioned herein, in one or more embodiments, paramagnetic nanoparticles of the present application can allow for the effective delivery of nitro fatty acids. Nitro fatty acids have been shown to have significant therapeutic potential due to their efficacy both as potent, long-lasting anti-inflammatories and as anti-tumor agents. Prior to the present application, their therapeutic potential has been limited due to issues regarding how to delivery these materials to the target site.

In one or more embodiments, the present application provides for paramagnetic nanoparticles that can transport nitro fatty acids to the targeted site. As explained herein, paramagnetic nanoparticles derived from doped gadolinium oxide nanocrystals can be effectively coated with unsaturated fatty acids such as oleic acid and conjugated linoleic acid. A similar method is employed for coating the nanoparticles with nitro fatty acids. Specifically, the paramagnetic nanoparticles can be coated with nitro fatty acids by either converting a fatty acid coating to nitro fatty acids or using nitro fatty acids as starting material when coating the nanoparticles. Nitro fatty acids are generated by exposing the unsaturated fatty acid to a combination of nitric oxide and oxygen which produces NO₂, the free radical that drives the nitration process. In an alternative embodiment, nitro fatty acids can be directly incorporated into a paramagnetic hybrid-hydrogel nanoparticle platform based on silane plus chitosan derived hydrogels with dispersed gadolinium/europium hydroxide nanoclusters uniformly distributed throughout the hydrogel-based nanoparticles.

One method for preparing a paramagnetic hybrid-hydrogel nanoparticle of the present application comprises, for example: (a) hydrolyzing TMOS; (b) mixing the sol-gel (hydrogel) components; (c) lyophilizing the sol-gel; (d) ball-milling the lyophilized sol-gel particles; and (e) PEGylating the nanoparticles. Specifically, stock of 5 ml of TMOS, 600 μl of deioinized water, and 560 μl of 2 mM hydrochloric acid are added to a small vial. The contents of the vial are then sonicated approximately 20-30 minutes to get a clear solution and placed on ice. A separate solution of 800 mg of gadolinium chloride hexahydrate and 200 mg of europium chloride hexahydrate are then solubilized in 6-8 ml of deionized water followed by sequential addition and mixing of 1 ml of PEG-200, 1 ml (1 mg/ml) of either chitosan or water soluble chitosan (depending on the application and usage), and 30 ml of methanol. The resulting mixture is then vortexed thoroughly. Then, 2 ml of the previously hydrolyzed TMOS is added to the solution along with approximately 75-150 μl of 3-aminopropyltrimethoxysilane followed by constant stirring. 4 to 6 ml of ammonium hydroxide is added to the above admixture to form gel, followed by vigorous vortexing until complete gelation. The hydroxide creates paramagnetic gadolinium/europium hydroxide that is distributed throughout the resulting hydrogel. The hydroxide also accelerates polymerization which favors small polymers leading to smaller nanoparticles. The resulting gelled material is then lyophilized for 24-48 hours, which removes all volatile components including the methanol. Following lyophilization, the dry material is ball milled at 150 rpm for 8 hours. The resulting material is a very fine white powder. Finally, PEGylation of the paramagnetic nanoparticles is achieved by mixing a suspension of the nanoparticles with an amine-binding PEG. Similarly, peptides can be bound to the surface via reaction with the amines on the surface of the nanoparticle. This process can be carried out in water, alcohol or DMSO depending on the nature of the deliverable. Water will initiate release for nitric oxide, and thus in embodiments in which NO is included in the nanoparticle, the PEGylation needs to be carried out in DMSO, which does not result in release of NO. Once the reaction is complete, the PEGylated nanoparticles can be redried and then stored as a dry powder. The nanoparticle platform can be slightly altered depending on the desired properties and the materials to be loaded. For example, in an alternative embodiment, thiols can be incorporated into the nanoparticle by using thiol-containing silanes in a manner similar to the process of introducing amines. This approach allows covalent attachment of the silane hydrogel backbone thiol binding fluorescent probes such as BADAN.

In certain embodiments, modified paramagnetic nanoparticles of the present application can be utilized to treat patients with one or more diseases or disorders. In at least one embodiment, a patient is administered an effective amount of the modified paramagnetic nanoparticles and a magnetic field is then applied to the subject at the location of the disease or disorder (e.g., inflammation) such that the magnetic field is at sufficient strength to attract the nanoparticles to the location of the disease or disorder.

A method for preparing a hybrid-hydrogel nitro oxide-releasing nanoparticle with added conjugated linoleic acid comprises, for example: (a) hydrolyzing TMOS; (b) mixing the sol-gel components; (c) lyophilizing the sol-gel; and (d) ball-milling the sol-gel particles. Specifically, 5 ml of TMOS, 600 μl of deioinized water, and 560 μl of 2 mM hydrochloric acid are added to a small vial. The contents of the vial are then sonicated approximately 20-30 minutes to get a clear solution and placed on ice. 1 ml of conjugated linoleic acid (sigma) in DMSO (1:19 v/v ratio in stock), 1.49 g of sodium nitrite (dissolved in 4 ml of PBS buffer at pH 7.5), 1 ml of PEG-200, 800 μl of chitosan (1 mg/ml), and 28 ml of methanol are then mixed in the above order and vortexed thoroughly. Then, 2 ml of previously hydrolyzed TMOS is added to the solution, and 50-75 μl of 3-aminopropyltrimethoxysilane is added followed by vigorous vortexing until complete gelation. The gel was then lyophilized for 24-48 hrs, and the resulting particles were ball milled at 150 rpm for 8 hours.

A method for preparing a hybrid-hydrogel nitric oxide-releasing nanoparticle with a polyvinyl acid additive comprises, for example: (a) hydrolyzing TMOS; (b) mixing the sol-gel components; (c) washing the sol-gel; (d) lyophilizing the sol-gel; and (e) ball-milling the sol-gel particles. Specifically, 5 ml of TMOS, 600 μl of deioinized water, and 560 μl of 2 mM hydrochloric acid are added to a small vial. The contents of the vial are then sonicated approximately 20-30 minutes to get a clear solution and placed on ice. 28 ml of methanol, 1 mL of polyvinyl alcohol (PVA) from stock solution (10 mg/mL in deionized water), 2 ml of 300 mM Tris (HCl) buffer at pH 7.5, 1 ml of glycerol, 4 ml of chitosan (1 mg/ml), and 2.76 g of sodium nitrite are then dissolved in the mixture in the above order, and vortexed thoroughly. Then, 4 ml of previously hydrolyzed TMOS is added to the tube, and the contents are vortexed for about two minutes. The tube is allowed to sit undisturbed for gelation. It forms gel in 5 to 10 min. The resulting sol-gel is crushed and deionized water is added until the tube is nearly full. The contents are then vortexed until the mixture is relatively homogeneous. Then, the mixture is centrifuged at 6,000 rpm for 25 minutes, and the supernatant is removed. The gel is then lyophilized for 24-48 hrs. Finally, the resulting particles were ball milled at 150 rpm for 3 hours.

In another aspect, the present application provides for a method of enhancing of nitric oxide (NO) levels in the body via the use of hybrid-hydrogel based nanoparticles prepared with NO-responsive fluorophores (e.g., diamino fluorescein [DAF]). NO is a critically important part of innumerable physiological processes. As such, systemic and targeted delivery of NO as a therapeutic modality is an important and timely biomedical objective. Further, it is important to monitor NO levels in response to administration of therapeutics that are designed to enhance NO levels in specific tissues. For example, in pursuit of strategies for topical administration of vehicles such as NO-releasing nanoparticles and other NO releasing or producing agents, it is of importance to be able to monitor the enhancement of NO levels as a function of skin depth to assess penetration. This information is particularly critical with respect to developing topical treatments for peripheral vascular disease and erectile dysfunction.

Continuing with this aspect of the present application, hybrid-hydrogel based nanoparticles can be prepared with NO-responsive fluorophores, which undergo a several order magnitude enhancement in fluorescence when they react with NO. These loaded nanoparticles can either be optimized for maximum skin penetration or injected at multiple depths. The high local concentration of the probe containing the NO-responsive fluorophore within each nanoparticle will provide a significant advantage of the free fluorophore with respect to detecting NO at varying depths below the skin. Skin biopsies followed by evaluation in a fluorescence microscope can be used to assess the NO levels. Additionally the nanoparticles can be further modified with a second fluorescent probe (different emission wavelength) to provide a clear picture of where the nanoparticles are localized. In an alternative embodiment, the NO-responsive fluorophores can be applied to gadolinium-based paramagnetic nanoparticles, where the probe molecules containing the NO-responsive fluorophores can be loaded in a fatty acid coating of the gadolinium oxide core. This strategy would allow for magnetic localization of the systemically administered paramagnetic nanoparticles at target sites not accessible by topical delivery. Whole body fluorescence imaging can be used to follow the build of NO at the targeted site (e.g. tumor, localized inflammation, vascular obstruction, etc.).

In at least one aspect, the present application also provides for an NO-releasing nanoparticle that facilitates transnitrosylation. In particular, in one or more embodiments, the nanoparticle generates and releases NO, and incorporates an angiotensin converting enzyme inhibitor (ACE), captopril. Captopril contains a thiol group that can be nitrosylated to form S-nitrosocaptopril (SNO-CAP). SNO-CAP itself can have potent vasodilating and antiplatelet effect, and can maintain its ability to inhibit ACE. Thus, in at least one embodiment, the present application provides for a SNO-CAP nanoparticle. In this embodiment, as NO is generated and released from the SNO-CAP-containing nanoparticles, it is bound up by the captopril sulfhydryl moiety, providing a long lasting NO-donating technology. At the nanoscale, this technology has an increased ability to interact with its intended target and exert its biological impact over an extended period of time.

The SNO-CAP nanoparticles (SNO-CAP-np) of the present application have many therapeutic applications, including but not limited to sustained nitrosylation activity (e.g., via production of S-nitrosoglutathione [GSNO] in the presence of glutathione [GSH]), and antimicrobial activity against E. coli and MRSA.

### 5.2 Curcumin-Encapsulated Nanoparticles

In at least one aspect, the present application also provides for a curcumin-encapsulated nanoparticle. In another aspect, the present application provides for a curcumin-based composition. In one or more embodiments, the curcumin composition and the curcumin-encapsulated nanoparticle are treatments for dermatophytic fungi. Dermatophytic fungi utilize nutrients from keratinized tissue, such as skin, hair and nails, and are the etiologic agents of superficial skin mycoses, known as dermatophytoses. Given the superficial nature of these infections and ease of access by a light source, there has been renewed focus on antimicrobial photodynamic inhibition (aPI). aPI is a technique that generates reactive oxygen and nitrogen species by exciting a pharmacologically inert photosensitizer (PS) with light matched to its absorption wavelength, in the presence of oxygen. One such PS is curcumin (diferuloylmethane), which is a yellow crystalline compound isolated from the spice, turmeric. Curcumin absorbs in the 408-434 nm range, generally requiring blue light for photoactivation, and has been shown to exert strong phototoxic effects against bacterial and fungal species. Curcumin is commercially available in highly purified form and exhibits low dark toxicity, properties essential for optimal photosensitization. However, its therapeutic translation has previously been limited by low oral bioavailability, poor aqueous solubility, and rapid degradation at physiologic pH, creating a formulation challenge.

In accordance with at least one embodiment of the present application, the encapsulation of curcumin in nanoparticles stabilizes curcumin from degradation and allows for suspension in an aqueous solvent. Liposomes, cyclodextrins and micelles have previously been investigated as solubilizers and nanocarriers of curcumin for aPI against bacterial species. However, these previous methods have been hindered by preferential attraction of curcumin to the carrier rather than microbial surfaces and temporal instability, and, therefore, decreased efficacy following preparation. In one aspect of the present application, a hydrophilic matrix, which swells to release curcumin in an aqueous environment, is incorporated in the nanoparticle to overcome these limitations. In accordance with one or more embodiments, the curcumin-based composition and the curcumin-encapsulated nanoparticle, both in combination with blue light doses (aPI) can inhibit the growth of dermatophytic fungi, as explained further in Section 6 (Examples).

In accordance with one or more embodiments, the present application also provides for curcumin-encapsulated hybrid-hydrogel nanoparticles. In one or more embodiments, the curcumin curcumin-encapsulated hybrid-hydrogel nanoparticles are treatments for infected burn wounds. Among traumatic injuries, burns represent a significant source of morbidity and mortality. The curcumin-encapsulated hybrid-hydrogel nanoparticles, in accordance with one or more embodiments, exhibit antimicrobial activity against P. aeruginosa and MRSA, as further explained in Section 6 (Examples). Curcumin-encapsulated nanoparticles, in accordance with one or more embodiments, also facilitate improvements in osteoarthritis-related endpoints.

### 5.3 Composition Comprising Modified Nanoparticles

In certain embodiments, the modified nanoparticles of the present application can be incorporated into one or more compositions. These compositions can contain a therapeutically effective amount of a modified nanoparticle, optionally more than one modified nanoparticle, preferably in purified form, together with a suitable amount of a pharmaceutically acceptable vehicle so as to provide the form for proper administration to the patient. In certain embodiments, the composition contains 1-5%, 5-10%, 10-20%, 20-30%, 30-40% modified nanoparticle.

In certain embodiments, the modified nanoparticles are administered to a subject using a therapeutically effective regimen or protocol. In certain embodiments, the modified nanoparticles are also prophylactic agents. In certain embodiments, the modified nanoparticles are administered to a subject or patient using a prophylactically effective regimen or protocol.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. In certain embodiments, an elderly human, human adult, human child, human infant. The term “vehicle” refers to a diluent, adjuvant, excipient, or carrier with which a compound of the present application is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the modified nanoparticles and pharmaceutically acceptable vehicles are preferably sterile. Water is a preferred vehicle when the modified nanoparticle is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions comprising the modified nanoparticles, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. Other examples of suitable pharmaceutical vehicles are described in “Remington's Pharmaceutical Sciences” by E. W. Martin.

In a preferred embodiment, the compounds of the present application are formulated in accordance with routine procedures as a pharmaceutical composition adapted for intravenous administration to human beings. Typically, compounds of the present application for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the present application is to be administered by infusion, it can be dispensed, for example, with an infusion bottle containing sterile pharmaceutical grade water or saline. Where the modified PMNP is administered by injection, an ampoule of sterile water for injection or saline can be provided so that the ingredients may be mixed prior to administration.

Compositions for oral delivery may be in the form of tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds of the present application. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

### 5.4 Types of Disease and Disorders

The present disclosure provides methods of treating or preventing or managing a disease or disorder in humans by administering to humans in need of such treatment or prevention a pharmaceutical composition comprising an amount of modified nanoparticles effective to treat or prevent the disease or disorder. In other embodiments, the disease or disorder is an inflammatory disease or disorder.

The present application encompasses methods for preventing, treating, managing, and/or ameliorating an inflammatory disorder or one or more symptoms thereof as an alternative to other conventional therapies. In specific embodiments, the patient being managed or treated in accordance with the methods of the present application is refractory to other therapies or is susceptible to adverse reactions from such therapies. The patient may be a person with a suppressed immune system (e.g., post-operative patients, chemotherapy patients, and patients with immunodeficiency disease, patients with broncho-pulmonary dysplasia, patients with congenital heart disease, patients with cystic fibrosis, patients with acquired or congenital heart disease, and patients suffering from an infection), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, manage, treat, or ameliorate a viral respiratory infection or one or more symptoms thereof.

In certain embodiments, the present application provides a method of preventing, treating, managing, and/or ameliorating an autoimmune disorder or one or more symptoms thereof, said method comprising administering to a subject in need thereof a dose of an effective amount of one or more pharmaceutical compositions of the present application. In autoimmune disorders, the immune system triggers an immune response and the body's normally protective immune system causes damage to its own tissues by mistakenly attacking self. There are many different autoimmune disorders which affect the body in different ways. For example, the brain is affected in individuals with multiple sclerosis, the gut is affected in individuals with Crohn's disease, and the synovium, bone and cartilage of various joints are affected in individuals with rheumatoid arthritis. As autoimmune disorders progress, destruction of one or more types of body tissues, abnormal growth of an organ, or changes in organ function may result. The autoimmune disorder may affect only one organ or tissue type or may affect multiple organs and tissues. Organs and tissues commonly affected by autoimmune disorders include red blood cells, blood vessels, connective tissues, endocrine glands (e.g., the thyroid or pancreas), muscles, joints, and skin.

Examples of autoimmune disorders that can be prevented, treated, managed, and/or ameliorated by the methods of the present application include, but are not limited to, adrenergic drug resistance, alopecia areata, ankylosing spondylitis, antiphospholipid syndrome, autoimmune Addison's disease, autoimmune diseases of the adrenal gland, allergic encephalomyelitis, autoimmune hemolytic anemia, autoimmune hepatitis, autoimmune inflammatory eye disease, autoimmune neonatal thrombocytopenia, autoimmune neutropenia, autoimmune oophoritis and orchitis, autoimmune thrombocytopenia, autoimmune thyroiditis, Behcet's disease, bullous pemphigoid, cardiomyopathy, cardiotomy syndrome, celiac sprue-dermatitis, chronic active hepatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, dense deposit disease, discoid lupus, essential mixed cryoglobulinemia, fibromyalgia-fibromyositis, glomerulonephritis (e.g., IgA nephrophathy), gluten-sensitive enteropathy, Goodpasture's syndrome, Graves' disease, Guillain-Barre, hyperthyroidism (i.e., Hashimoto's thyroiditis), idiopathic pulmonary fibrosis, idiopathic Addison's disease, idiopathic thrombocytopenia purpura (ITP), IgA neuropathy, juvenile arthritis, lichen planus, lupus erythematosus, Meniere's disease, mixed connective tissue disease, multiple sclerosis, Myasthenia Gravis, myocarditis, type 1 or immune-mediated diabetes mellitus, neuritis, other endocrine gland failure, pemphigus vulgaris, pernicious anemia, polyarteritis nodosa, polychrondritis, Polyendocrinopathies, polyglandular syndromes, polymyalgia rheumatica, polymyositis and dermatomyositis, post-MI, primary agammaglobulinemia, primary biliary cirrhosis, psoriasis, psoriatic arthritis, Raynauld's phenomenon, relapsing polychondritis, Reiter's syndrome, rheumatic heart disease, rheumatoid arthritis, sarcoidosis, scleroderma, Sjogren's syndrome, stiff-man syndrome, systemic lupus erythematosus, takayasu arteritis, temporal arteritis/giant cell arteritis, ulcerative colitis, urticaria, uveitis, Uveitis Opthalmia, vasculitides such as dermatitis herpetiformis vasculitis, vitiligo, and Wegener's granulomatosis.

Any type of cancer can be prevented, treated, and/or managed in accordance with one or more embodiments of the present application. Non-limiting examples of cancers that can be prevented, treated, and/or managed in accordance with the present application include: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; dendritic cell cancer, including plasmacytoid dendritic cell cancer, NK blastic lymphoma (also known as cutaneous NK/T-cell lymphoma and agranular (CD4+/CD56+) dermatologic neoplasms); basophilic leukemia; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

The prophylactically and/or therapeutically effective regimens are also useful in the treatment, prevention and/or management of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. In some embodiments, cancers associated with aberrations in apoptosis are prevented, treated and/or managed in accordance with the methods of the present application. Such cancers may include, but not be limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders of the skin, lung, liver, bone, brain, stomach, colon, breast, prostate, bladder, kidney, pancreas, ovary, and/or uterus are prevented, treated and/or managed in accordance with the methods of the present application. In other specific embodiments, a sarcoma, melanoma, or leukemia is prevented, treated and/or managed in accordance with the methods of the present application. In certain embodiments, the subjects have acute myelogenous leukemia (AML). In certain other embodiments, the subjects have myelodysplastic syndrome (MDS). In other embodiments, the subjects have chronic myelomonocytic leukemia (CMML). In other specific embodiments, myelodysplastic syndrome is prevented, treated and/or managed in accordance with the methods of the present application.

### 5.4.1 Cancer Treatment

A major objective in treatment of cancers is to be able to target the tumor with sufficient levels of the appropriate therapeutic without systemic toxicity. The use of targeting molecules attached to either the therapeutic molecules directly or to nanoparticles containing the therapeutic molecule has not proven to be especially effective. A major pathway for localization of either the free therapeutic molecule or the drug delivery vehicle containing the therapeutic molecule is through the EPR effect (EPR=enhanced permeability and retention) resulting from the leaky vasculature associated with many (but not all) tumors. For the EPR effect to work the circulating drug or delivery vehicle must remain in a functional form in circulation for a sufficiently long time to allow for the build of local concentration at the tumor site via the EPR effect. This build up requires circulation times of at least 8 to 24 hours. Thus, over this several hour period, a drug-loaded nanoparticle has to both avoid being cleared and avoid releasing its therapeutic payload (resulting in potential systemic effects and decreased efficacy at the target site). Herein is disclosed an approach and a biocompatible nanoparticle platform that takes advantage of the EPR effect but drastically shortens the accumulation time from hours to minutes. Drug-loaded paramagnetic nanoparticles (PMNP) (e.g. gadolinium oxide-based) are infused intravenously and then localized at the target site using a strategically placed external magnetic field. Based on imaging studies (both MRI and whole body fluorescence), a several minute treatment with the externally placed magnetic field is sufficient to create persistent localization for many hours once the magnetic field is removed. The persistent retention only occurs for those tissues manifesting the EPR effect. This approach when applied to targeting one of many xenographed tumors with adriamycin-loaded PMNPs results in rapid and effective site specific reduction in tumor size without evidence of either systemic toxicity or tumor shrinkage in non-targeted tumors. The ability to easily modify the PMNP platform to accommodate a wide variety of chemotherapeutic and immunogenic molecules as well cell-specific targeting molecules (peptides, antibodies, bisphosphonates, aptamers), makes this very powerful. Also, the induction of leaky vasculature in EPR resistant tumors through targeted treatments with radiation will likely make these resistant tumors accessible to this approach.

Targeted drug delivery using nanoparticles is a major trend in cancer therapy. Targeted delivery can be expected to minimize systemic toxicity and enhance efficacy by being able to deliver much larger doses of chemotherapeutic drugs directly to the site of the tumor. Tumor targeting using nanoparticles coated with targeting molecules is not very effective in vivo in part due to plasma proteins adhering to the nanoparticles and interfering with the range of motions or accessibility of the targeting molecules. Instead the most promising approaches appear based on utilizing the EPR effect (enhanced penetration and perfusion) arising from the leaky vasculature associated with many tumor types. For those tumors without such vessels, radiation induced inflammation can be used to create “leakiness” and thus render such tumors susceptible to the EPR effect. The EPR effect allows for localized accumulation of circulating nanoparticles over a period of many hours during which time the nano's have to remain in circulation and not release their drug payload. This requirement poses a serious challenge for the design of suitable platforms. This laboratory has shown that the use of paramagnetic nanoparticles (PMNPs) allows for very rapid accumulation of the PMNP's at the tumor site targeted using an externally applied magnetic field. Once initially localized using the external magnetic field, the PMNP's remain trapped for what may well be an indefinite period (at least 24 hours) after the magnetic field is removed. Thus the several hour accumulation time is reduced to minutes using the external magnetic field which can then be removed without concern that the PMNP's will continue to circulate. The PMNP's do not appear to permanently (or even transiently) accumulate in tissues that do not have the leaky vasculature (with or without the externally applied magnetic field). In contrast, the PMNP's do appear to accumulate in EPR sensitive tissues even in the absence of the magnetic field but instead of minutes the accumulation time is much longer as anticipated from many studies on the EPR effect using other types of nanoparticles. Albumin-based nanoparticle appear to be a promising strategy that utilizes the EPR effect. Abraxane is a notable example whereby taxol loaded albumin nanoparticles diminish systemic effects and appear to enhance efficacy by preferentially accumulating in the tumor. Building upon all of the above concepts by developing a general platform that allows for the coating of PMNS's with drug loaded albumin thereby adding the following capabilities and advantages: i) very rapid targeting/localization; ii) imaging; iii) enhanced and more efficient drug loading; and iv) greater plasticity with respect to drugs, combination of drugs and physical properties of the nanoparticles.

Albumin forms a very tight shell/coating around a gadolinium oxide core PMNPs that remains intact in aqueous solutions. Several drugs (curcumin, Adriamycin but not taxol) directly bind to the surface of the PMNP's with high avidity. Albumin can coat the drug loaded PMNP's. Albumin is an effective carrier/transporter for many lipophilic drugs hence both the PMNP and the albumin can be used to carry drugs. Taxol loaded albumin (Abraxane) can be used to coat the PMNP's thus allowing for taxol and related drugs to participate in the targeted delivery. PEG can easily be attached to the surface of the PMNP using PEG-DSPE (1,2-distearoyl-sn-glycero-3-phosphoethanolamine-N-[amino(polyethylene glycol)-2000) derivative. The DSPE moiety has a very high electrostatic attraction for the surface of the gadolinium oxide (GdO) nanoparticles. PEG imparts a stealth quality to nanoparticles allowing them to evade scavenging by macrophages. PEG also enhances the EPR effect making capture in leaky vessels more probable. Bifunctional PEG with one end having the DSPE moiety and the other end a reactive species (e.g. maleimide, amine, thiol) can be used to attach to the PMNP's PEG with fluorophores, PET imaging agents, peptides, antibodies, aptamers, and additional MRI contrast agents (the GdO based PMNPs have intrinsic relaxativity properties that can be tuned and used for positive contrast MRI imaging).

In certain embodiments, the method of treating cancer includes: (i) a reduction of cancer cells, (ii) absence of increase of cancer cells; (iii) a decrease in viability of cancer cells; (iv) decrease in growth of cancer cells, in a subject.

In certain embodiments, the subject that is treated with the present method of the disclosure has been diagnosed with the disease and has undergone therapy. In certain embodiments, the subject that is treated with the present method of the disclosure has been diagnosed with cancer and has undergone cancer therapy.

In certain embodiments, the subject is in remission from cancer. In certain embodiments, the subject has relapsed from cancer. In certain embodiments, the subject has failed cancer treatment.

### 5.5 Mode of Administration

The present compositions, which comprise one or more modified nanoparticles, can be administered by infusion or bolus injection, by absorption through epithelial or mucocutaneous linings (e.g., oral mucosa, rectal and intestinal mucosa, etc.) or orally and may be administered together with another biologically active agent. Administration can be systemic or local. Various delivery systems are known. In certain embodiments, more than one modified nanoparticle is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the modified nanoparticle into the bloodstream.

In specific embodiments, it may be desirable to administer one or more compounds of the present application locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site).

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the present application can be formulated as a suppository, with traditional binders and vehicles such as triglycerides.

In yet another embodiment, the compounds of the present application can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the modified nanoparticle, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

### 5.6 Dosage

The amount of a modified nanoparticle that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.001 milligram to 200 milligrams of a compound of the present application per kilogram body weight. In specific preferred embodiments of the present application, the oral dose is 0.01 milligram to 70 milligrams per kilogram body weight, more preferably 0.1 milligram to 50 milligrams per kilogram body weight, more preferably 0.5 milligram to 20 milligrams per kilogram body weight, and yet more preferably 1 milligram to 10 milligrams per kilogram body weight. In another embodiment, the oral dose is 5 milligrams of modified nanoparticle per kilogram body weight. The dosage amounts described herein refer to total amounts administered; that is, if more than one modified nanoparticle is administered, the preferred dosages correspond to the total amount of the modified nanoparticles administered. Oral compositions preferably contain 10% to 95% active ingredient by weight.

Suitable dosage ranges for intravenous (i.v.) administration are 0.01 milligram to 100 milligrams per kilogram body weight, 0.1 milligram to 35 milligrams per kilogram body weight, and 1 milligram to 10 milligrams per kilogram body weight. Suitable dosage ranges for intranasal administration are generally about 0.01 pg/kg body weight to 1 mg/kg body weight. Suppositories generally contain 0.01 milligram to 50 milligrams of modified nanoparticles per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of the modified nanoparticles for topical administration are in the range of 0.001 milligram to 1 milligram, depending on the area to which the compound is administered. Effective doses may be extrapolated from dose-response curves derived from in vitro or animal model test systems. Such animal models and systems are well known in the art.

The present application also provides pharmaceutical packs or kits comprising one or more containers filled with one or more modified nanoparticles. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one modified nanoparticles. In another embodiment, the kit comprises a modified nanoparticles and a second therapeutic agent.

The modified nanoparticles are preferably assayed in vitro and in vivo, for the desired therapeutic or prophylactic activity, prior to use in humans. For example, in vitro assays can be used to determine whether administration of a specific modified nanoparticle or a combination of modified nanoparticles is preferred for lowering fatty acid synthesis. The modified nanoparticles may also be demonstrated to be effective and safe using animal model systems.

Other methods will be known to the skilled artisan and are within the scope of the present application.

### 5.7 Combination Therapy

In certain embodiments, the modified nanoparticles of the present application can be used in combination therapy with at least one other therapeutic agent. The modified nanoparticles and the therapeutic agent can act additively or, more preferably, synergistically. In a preferred embodiment, a composition comprising a modified nanoparticle is administered concurrently with the administration of another therapeutic agent, which can be part of the same composition as the modified nanoparticle or a different composition. In another embodiment, a composition comprising a modified nanoparticle is administered prior or subsequent to administration of another therapeutic agent. As many of the disorders for which the modified nanoparticles are useful in treating are chronic disorders, in one embodiment combination therapy involves alternating between administering a composition comprising a modified nanoparticle and a composition comprising another therapeutic agent, e.g., to minimize the toxicity associated with a particular drug. The duration of administration of each drug or therapeutic agent can be, e.g., one month, three months, six months, or a year. In certain embodiments, when a modified nanoparticle is administered concurrently with another therapeutic agent that potentially produces adverse side effects including but not limited to toxicity, the therapeutic agent can advantageously be administered at a dose that falls below the threshold at which the adverse side is elicited.

In certain embodiments, the modified nanoparticles of the present application can be administered together with one or more antifungal agents in the form of antifungal cocktails, or individually, but close enough in time to have a synergistic effect on the treatment of the infection. An antifungal cocktail is a mixture of any one of the compounds described herein with another antifungal drug. In one embodiment, a common administration vehicle (e.g., tablet, implants, injectable solution, injectable liposome solution, etc.) is used in for the compound as described herein and other antifungal agent(s).

Anti-fungal agents are useful for the treatment and prevention of infective fungi. Anti-fungal agents can be classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

Other antifungal agents include Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Cancidas (Caspofungin Acetate), Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofungin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafungin; Undecylenic Acid; Viridofulvin; Zinc Undecylenate; and Zinoconazole Hydrochloride.

In certain embodiments, the modified nanoparticles described herein can be used in combination with one or more antifungal compounds. These antifungal compounds include but are not limited to: polyenes (e.g., amphotericin b, candicidin, mepartricin, natamycin, and nystatin), allylamines (e.g., butenafine, and naftifine), imidazoles (e.g., bifonazole, butoconazole, chlordantoin, flutrimazole, isoconazole, ketoconazole, and lanoconazole), thiocarbamates (e.g., tolciclate, tolindate, and tolnaftate), triazoles (e.g., fluconazole, itraconazole, saperconazole, and terconazole), bromosalicylchloranilide, buclosamide, calcium propionate, chlorphenesin, ciclopirox, azaserine, griseofulvin, oligomycins, neomycin undecylenate, pyrrolnitrin, siccanin, tubercidin, and viridin. Additional examples of antifungal compounds include but are not limited to Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofingin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafuigin; Undecylenic Acid; Viridoflilvin; Zinc Undecylenate; and Zinoconazole Hydrochlorid

In certain embodiments, the modified nanoparticles of the present application can be administered together with treatment with irradiation or one or more chemotherapeutic agents. For irradiation treatment, the irradiation can be gamma rays or X-rays. For a general overview of radiation therapy, see Hellman, Chapter 12: Principles of Radiation Therapy Cancer, in: Principles and Practice of Oncology, DeVita et al., eds., 2.nd. Ed., J. B. Lippencott Company, Philadelphia. Useful chemotherapeutic agents include methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a specific embodiment, a composition comprising the modified nanoparticle further comprises one or more chemotherapeutic agents and/or is administered concurrently with radiation therapy. In another specific embodiment, chemotherapy or radiation therapy is administered prior or subsequent to administration of a present composition, preferably at least an hour, five hours, 12 hours, a day, a week, a month, more preferably several months (e.g., up to three months), subsequent to administration of a composition comprising the modified nanoparticle.

Any therapy (e.g., therapeutic or prophylactic agent) which is useful, has been used, or is currently being used for the prevention, treatment, and/or management of a disorder, e.g., cancer, can be used in compositions and methods of the present application. Therapies (e.g., therapeutic or prophylactic agents) include, but are not limited to, peptides, polypeptides, conjugates, nucleic acid molecules, small molecules, mimetic agents, synthetic drugs, inorganic molecules, and organic molecules. Non-limiting examples of cancer therapies include chemotherapies, radiation therapies, hormonal therapies, and/or biological therapies/immunotherapies and surgery. In certain embodiments, a prophylactically and/or therapeutically effective regimen of the present application comprises the administration of a combination of therapies.

Examples of cancer therapies include, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; EphA2 inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; anti-CD2 antibodies; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other examples of cancer therapies include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; Bcl-2 inhibitors; Bcl-2 family inhibitors, including ABT-737; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; vorozole; zanoterone; zeniplatin; zilascorb; and zinostatin stimalamer.

In some embodiments, the therapy(ies) used in combination with the modified nanoparticles is an immunomodulatory agent. Non-limiting examples of immunomodulatory agents include proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide). Other examples of immunomodulatory agents can be found, e.g., in U.S. Publ'n No. 2005/0002934 A1 at paragraphs 259-275 which is incorporated herein by reference in its entirety. In one embodiment, the immunomodulatory agent is a chemotherapeutic agent. In an alternative embodiment, the immunomodulatory agent is an immunomodulatory agent other than a chemotherapeutic agent. In some embodiments, the therapy(ies) used in accordance with the present application is not an immunomodulatory agent.

In some embodiments, the therapy(ies) used in combination with the modified nanoparticles is an anti-angiogenic agent. Non-limiting examples of anti-angiogenic agents include proteins, polypeptides, peptides, conjugates, antibodies (e.g., human, humanized, chimeric, monoclonal, polyclonal, Fvs, ScFvs, Fab fragments, F(ab)2 fragments, and antigen-binding fragments thereof) such as antibodies that bind to TNF-alpha, nucleic acid molecules (e.g., antisense molecules or triple helices), organic molecules, inorganic molecules, and small molecules that reduce or inhibit angiogenesis. Other examples of anti-angiogenic agents can be found, e.g., in U.S. Publ'n No. 2005/0002934 A1 at paragraphs 277-282, which is incorporated by reference in its entirety. In other embodiments, the therapy(ies) used in accordance with the present application is not an anti-angiogenic agent.

In some embodiments, the therapy(ies) used in combination with the modified nanoparticles is an inflammatory agent. Non-limiting examples of anti-inflammatory agents include any anti-inflammatory agent, including agents useful in therapies for inflammatory disorders, well-known to one of skill in the art. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide (ATROVENT™)), β2-agonists (e.g., abuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™)), and methylxanthines (e.g., theophylline (UNIPHYL™, THEO-DUR™, SLO-BID™, AND TEHO-42™)). Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), corticosteroids (e.g., methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (e.g., prostaglandins, thromboxanes, and leukotrienes. In other embodiments, the therapy(ies) used in accordance with the present application is not an anti-inflammatory agent.

In certain embodiments, the therapy(ies) used is an alkylating agent, a nitrosourea, an antimetabolite, and anthracyclin, a topoisomerase II inhibitor, or a mitotic inhibitor. Alkylating agents include, but are not limited to, busulfan, cisplatin, carboplatin, chlorambucil, cyclophosphamide, ifosfamide, decarbazine, mechlorethamine, melphalan, and themozolomide. Nitrosoureas include, but are not limited to carmustine (BCNU) and lomustine (CCNU). Antimetabolites include but are not limited to 5-fluorouracil, capecitabine, methotrexate, gemcitabine, cytarabine, and fludarabine. Anthracyclines include but are not limited to daunorubicin, doxorubicin, epirubicin, idarubicin, and mitoxantrone. Topoisomerase II inhibitors include, but are not limited to, topotecan, irinotecan, etoposide (VP-16), and teniposide. Mitotic inhibitors include, but are not limited to taxanes (paclitaxel, docetaxel), and the vinca alkaloids (vinblastine, vincristine, and vinorelbine).

In certain embodiments, the modified nanoparticles of the present application can be administered together with one or more antibiotic agents. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

In certain embodiments, modified nanoparticles are used in combination with topical agents that are contemplated to be selectably used for treatment of burns and wound healing. These topical agents can included, but are not limited to: albumin-based solutions, growth factors such as human recombinant epidermal growth factor, vascular endothelial growth factor, recombinant human basic fibroblast growth factor, keratocyte growth factor, platelet-derived growth factor, transforming growth factor beta, and nerve growth factor; anabolic hormones such as growth hormone and human insulin; any protease inhibitor such as nafamostat mesilate; any antibiotic compound at doses shown to safe and effective for human use such as a triple antibiotic (neomycin, polymyxin B, and bacitracin), neomycin, and mupirocin; and the gastric pentapeptide BPC 157.

In some embodiments, modified nanoparticle is used in combination with radiation therapy comprising the use of x-rays, gamma rays and other sources of radiation to destroy cancer stem cells and/or cancer cells. In specific embodiments, the radiation therapy is administered as external beam radiation or teletherapy, wherein the radiation is directed from a remote source. In other embodiments, the radiation therapy is administered as internal therapy or brachytherapy wherein a radioactive source is placed inside the body close to cancer stem cells, cancer cells and/or a tumor mass.

Currently available cancer therapies and their dosages, routes of administration and recommended usage are known in the art and have been described in such literature as the Physician's Desk Reference (60th ed., 2006). In accordance with the present application, the dosages and frequency of administration of chemotherapeutic agents are described supra.

### 6. EXAMPLES

The following example (Section 6.1) refers to the preparation and characterization of NO-releasing hybrid hydrogel nanoparticles in accordance with one or more embodiments of the present application.

### 6.1 Preparation and Characterization of NO-Releasing Hybrid Hydrogel Nanoparticles (NO-Np) with Both Alcohol and Added Aminosilane

In this example, the NO-np were prepared using the following sequence of steps. 1) Hydrolyzing Tetramethylorthosilicate (TMOS): Stock of 5 ml of TMOS, 600 μl of deioinized water, and 560 μl of 2 mM hydrochloric acid were added to a small vial. The contents of the vial were sonicated for approximately 20-30 minutes yielding a clear solution that was then placed on ice. 2) Mixing the sol-gel components: 1.49 g of sodium nitrite were dissolved in 4 ml of PBS buffer at pH 7.5 followed by sequential addition and mixing of 0.5 ml of PEG-200, 500 μl of chitosan (1 mg/ml), and 34 ml of methanol. The resulting mixture was then vortexed thoroughly. Then, 2 ml of previously hydrolyzed TMOS was added to the solution along with approximately 50-75 μl of 3-aminopropyltrimethoxysilane followed by vigorous vortexing until complete gelation. 3) Lyophilizing the sol-gel: The resulting gelled material was then lyophilized for 24-48 hrs which removed all volatile components including the methanol. 4) Ball-Milling the lyophilized sol-gel: Following lyophilization the dry material was ball milled at 150 rpm for 8 hours.

NO-Np Characterization of Platform with Alcohol and Added Aminosilane:

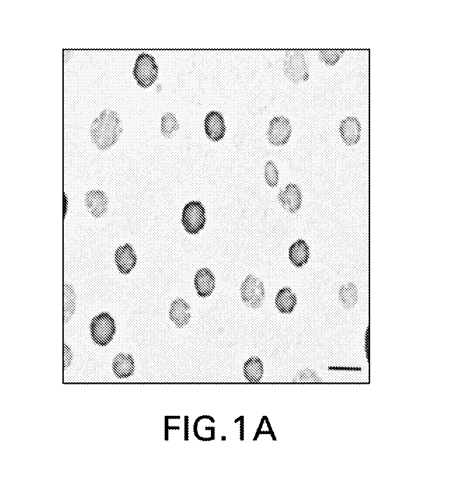

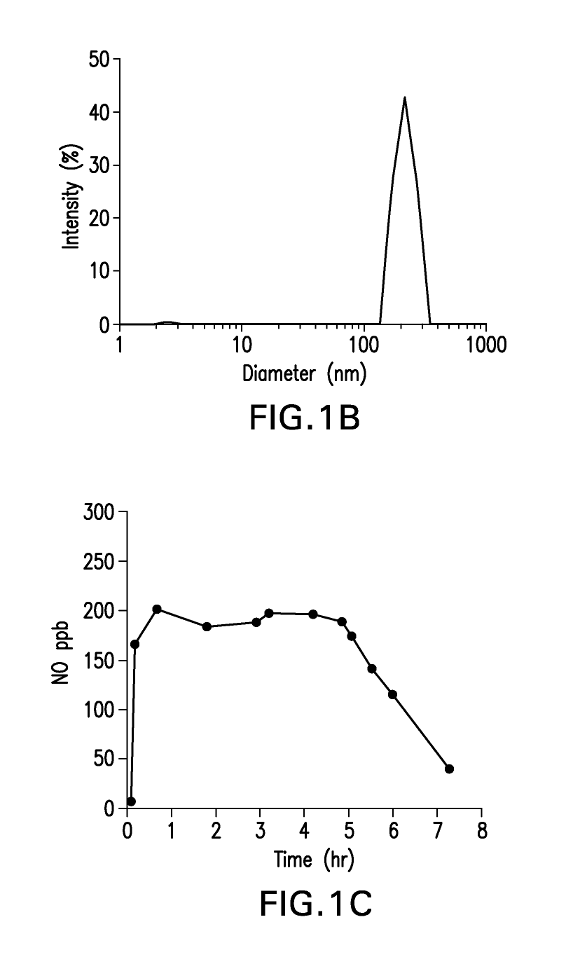

The resulting NO-np was a very fine white powder with no visible granularities. With scanning EM, results showed nanoparticles with a mean diameter of 55.6±14.8 nm (FIG. 1A). DLS analysis demonstrated a relatively narrow distribution of sizes for the NO-np, that is centered at 226.5 nm based on 40 acquisition attempts. The standard deviation is 8.9, showing that NO-np are homogenous in size. Since NO-np swell with moisture, the average diameter is likely an overestimate (FIG. 1B).

NO release from the NO-np is depicted in FIG. 1C. A peak release concentration was reached at 40.2 minutes, after which a steady state release ranging between 184-196 ppb NO was achieved, with subsequent decline of release rate extending to the end of the investigation at 7.2 hours. Measurements at lower pH values showed only very small changes in the releasing profiles, suggesting that very limited amounts of residual nitrite remain in the nanoparticles (nitrite converts to NO at low pH).

During the preparation (just prior to after gelation but prior to the lyophlization), evaluation of NO release via GSNO (the S-nitrosothiol derivative of glutathione) production from GSH (glutathione) showed no release of NO at this stage of preparation for the new platform when both alcohol and aminopropyltrimethoxysilane are used.

The following examples (Sections 6.2-6.7) refer to the preparation, characterization, efficacy and toxicity of S-nitrosocaptopril containing nanoparticles (SNO-CAP-np) in accordance with one or more embodiments of the present application.

### 6.2 Synthesis of SNO-CAP-Np Nanoparticles

In this example, a modified tetramethylorthosilicate (TMOS)-based sol-gel method was used to prepare SNO-CAP-np. Briefly, TMOS (3 mL) was hydrolyzed with 1 mM HCl (0.6 mL) by sonication on an ice-bath. The hydrolyzed TMOS (3 mL) was added to a buffer mixture of 1.5 mL of 0.5% chitosan, 1.5 mL of polyethylene glycol (PEG) 400, and 24 mL of 50 mM phosphate (pH 7.4) containing 0.225 M nitrite and 0.28 M captopril. The mixture was left at room temperature overnight in the dark for polymerization. A pink, opaque sol-gel formed, which was lyophilized and then ball milled in a Pulverisette 6 planetary ball-mill (Fritsch, Idar-Oberstein, Germany) into fine powder. The product was stored at −80° C. until use. In addition, nanoparticles synthesized for the in vivo toxicity assay also included nanoparticles without nitrite and captopril (control-np).

### 6.3 Size Characterization of SNO-CAP Nanoparticles

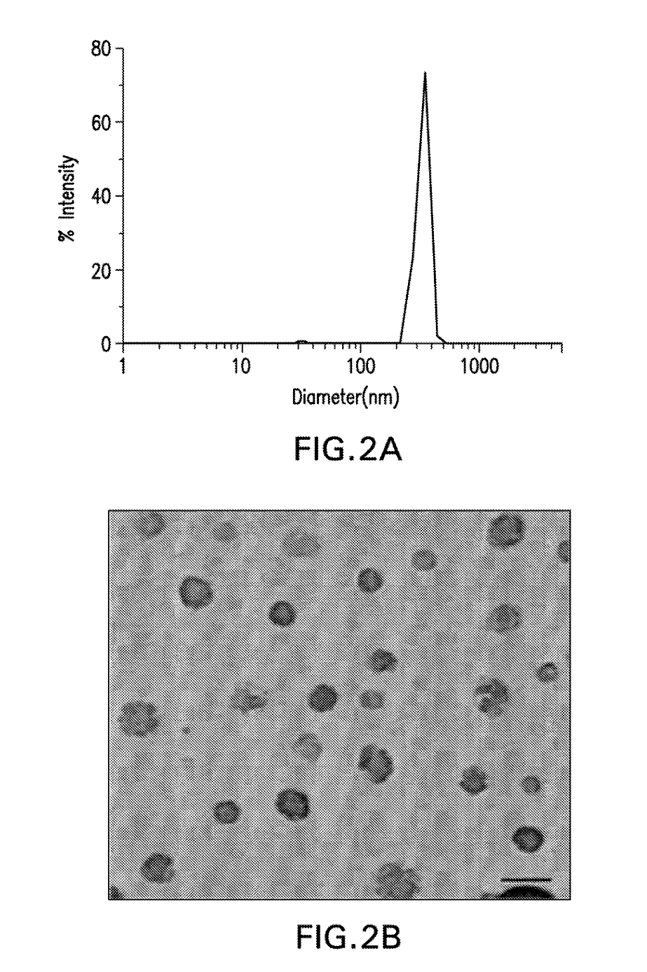

In this example, SNO-CAP-np size was determined by scanning electron microscopy (SEM), which was congruent with previous data in which our similarly-designed NO-np was measured via transmission electron microscopy (TEM). While previous TEM preparations were imaged to show individual nanoparticles of 10 nm in diameter, our current SEM preparations yielded nanoaggregates of 60-80 nm in diameter (measured from 100 nanoaggregates). However, individual nanoparticles could be visualized within many of the nanoaggregates which were also approximately 10 nm in diameter (FIG. 2B). The white scale bar represents 100 nm Dynamic light scattering (DLS) of 2.5 mg/mL SNO-CAP-np revealed an average hydrodynamic diameter of 377.8 nm based on 40 acquisition attempts (FIG. 2A). The standard deviation was 16.4 nm (4.3%), proving that Captopril-SNO-np are homogenous in size. Since SNO-CAP-np swell with moisture, the average diameter by DLS is likely an overestimation of their dry size, and is also likely to be a better approximation of their actual size in vivo.

### 6.4 NO Release Profile of SNO-CAP Nanoparticles

In this example, the time course of NO formation from SNO-CAP-np in PBS (1 mg/mL) was evaluated over 12 hours via chemiluminescent NO analyzer. More specifically, the rate of NO release from SNO-CAP-np was monitored using a chemiluminescent NO analyzer (Sievers NO analyzer, Model 280i, Boulder, Colo.). SNO-CAP-np were dispersed in 6 mL of PBS at 1 mg/mL concentration. This solution was continuously bubbled with pure nitrogen gas (0.2 L/min). The gas phase was collected into the NO analyzer and the signal was monitored via software.

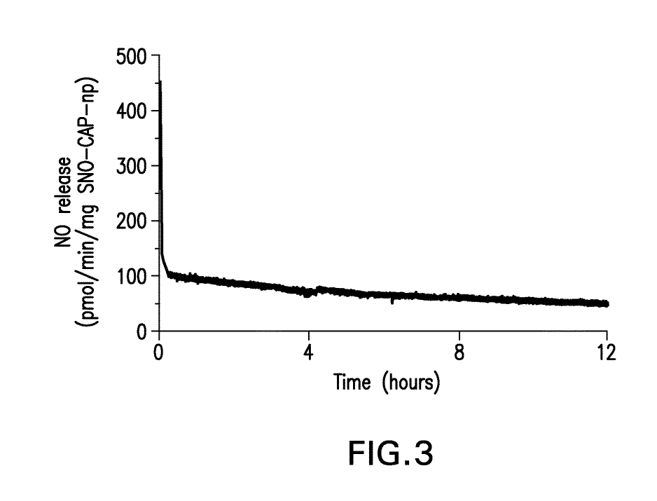

Within 2 minutes, the NO concentration peaked rapidly at 11.1 μM, and fell to levels below 4 μM after 4 minutes. NO concentration stabilized at about 2.4 μM after 19 minutes and decayed to a final concentration of about 1.2 μM after 12 h, thus demonstrating sustained NO release over at least 12 hours (FIG. 3).

### 6.5 GSNO Formation Reaction

In this example, SNO-CAP-np (20 mg/mL) were incubated with GSH (20 mM) during which aliquots were taken at 1, 30, 60, 120 and 240 minutes and characterized by RPHPLC. More specifically, SNO-CAP-np (20 mg/mL) were suspended in 20 mM GSH/0.5 mM EDTA/PBS solution at room temperature while mixing on a Lab Rotator shielded from light. At 1, 30, 60, 120 and 240 minutes, 10 μL aliquots were taken, diluted to 500 μL in 0.5 EDTA/PBS, and stored at −80° C. prior to RPHPLC analysis. Aliquots were also collected in the same fashion from a control suspension of SNO-CAP-np (20 mg/mL) in 0.5 mM EDTA/PBS.

RPHPLC analysis was performed with a Vydac 218TP C₁₈ equipped with a 5 μm analytical column (250 mm×4.6 mm, W.R. Grace & Co.-Conn., Columbia, Md.). Samples were run in an isocratic 10 mM dipotassium phosphate/10 mM tetrabutylammonium hydrogen sulfate, 5% acetonitrile buffer (pH 7.0) at a 0.5 mL/min flow rate, and were detected by UV absorbance at 210 nm Peak identities were confirmed by comparing the chromatogram of the GSNO formation reaction to chromatograms of the control reaction, as well as to individual chromatograms of GSH, GSNO, sodium nitrite, captopril, and SNO-CAP (non-np), diluted in 0.5 mM EDTA/PBS. GSNO concentrations were calculated by comparing peak areas during the GSNO formation reaction to the peak area of a GSNO standard of known concentration.

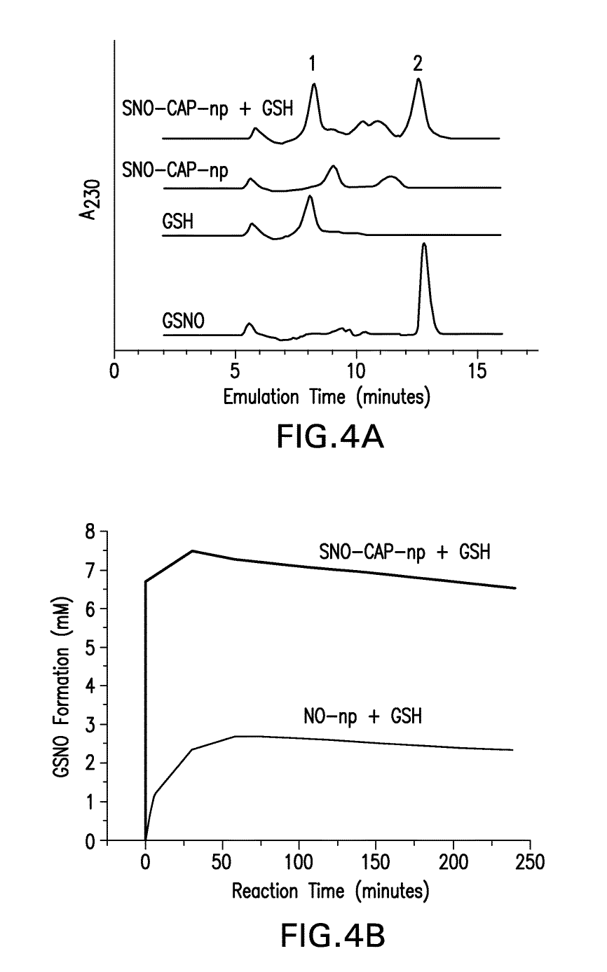

GSH and GSNO peaks (labeled 1 and 2) were identified in the chromatogram of the reaction mixture by comparing the individual components separately (FIG. 4A). Small unidentified peaks in the reaction mixture chromatogram are likely oxidized products of GSH and GSNO, such as glutathione disulfide (GSSG). Unreacted nitrite peaks were not found in the reaction mixture, as confirmed by RPHPLC analysis of sodium nitrite. Pure captopril and SNO-CAP (non-np) samples analyzed by RPHPLC did not yield any useful peaks, as we discovered that neither captopril nor SNO-CAP bound to the Vydac C18 column.

To demonstrate SNO-CAP-np transnitrosylation activity, the time course of GSNO formation from the SNO-CAP-np+GSH reaction mixture was determined by comparing the peak area of a GSNO standard to reaction mixture chromatograms at successive time points. Specifically, GSNO concentrations were plotted at 1, 30, 60 and 240 minutes (120 minute time point was omitted). The same data were also plotted for NO-np (20 mg/mL)+GSH (20 mM) to demonstrate SNO-CAP-np's increased transnitrosylation activity. GSNO formation by SNO-CAP-np in the presence of GSH was plotted alongside GSNO formation by NO-np in the presence of GSH, and SNO-CAP-np demonstrated more than 2.5-fold greater transnitrosylation activity compared to NO-np for the same concentration of nanoparticles (20 mg/mL). For SNO-CAP-np, the formation of GSNO levels greater than 6.5 mM was instantaneous, and reached peak levels of 7.49 mM GSNO within 30 minutes. In comparison, NO-np reached peak levels of 2.74 mM GSNO within 60 minutes. Transnitrosylation activity by SNO-CAP-np maintained relatively constant levels of GSNO for at least 4 h (FIG. 4B).

### 6.6 Susceptibility of E. coli and MRSA to SNO-CAP Nanoparticles and Captopril

In this example, 8 clinical strains each of MRSA and E. coli were evaluated. For each bacterial strain, one colony of bacteria grown on tryptic soy agar (TSA) was suspended in 1 mL tryptic soy broth (TSB). One μL aliquots were transferred to a 100-well honeycomb plate with 199 μL TSB. This TSB contained either 1, 2.5, 5, or 10 mg/mL SNO-CAP-np, or 2.5, 5, or 10 mM captopril, and controls included wells containing bacteria and TSB alone. The background absorbance of each SNO-CAP-np concentration was accounted for by wells containing SNO-CAP-np and TSB alone. No background absorbance was measured for captopril. Prior to plating, all SNO-CAP-np concentrations were sonicated for 1 minute on ice with a Fisher Sonic Dismembrator (model 100, Fisher Scientific, Pittsburgh, Pa.) to disperse the particles. All wells were incubated for 24 hours at 37° C. and growth was assessed by measuring optical density at 600 nm (0D600) with a microplate reader (Bioscreen C, Growth Curves USA, Piscataway, N.J.). Each condition was measured in triplicate, and averages were calculated along with standard error of the mean (SEM).

SNO-CAP-Np Inhibits E. coli and MRSA Growth.

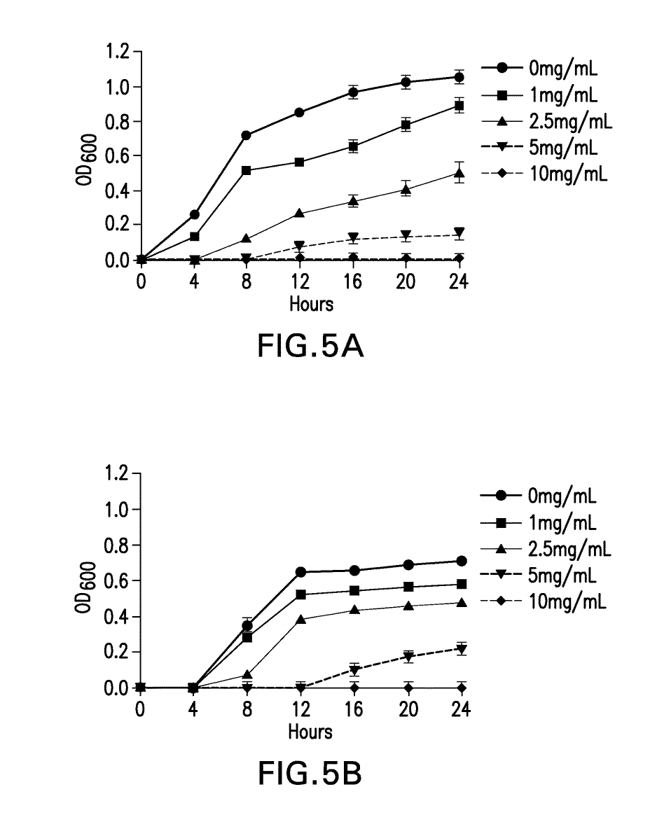

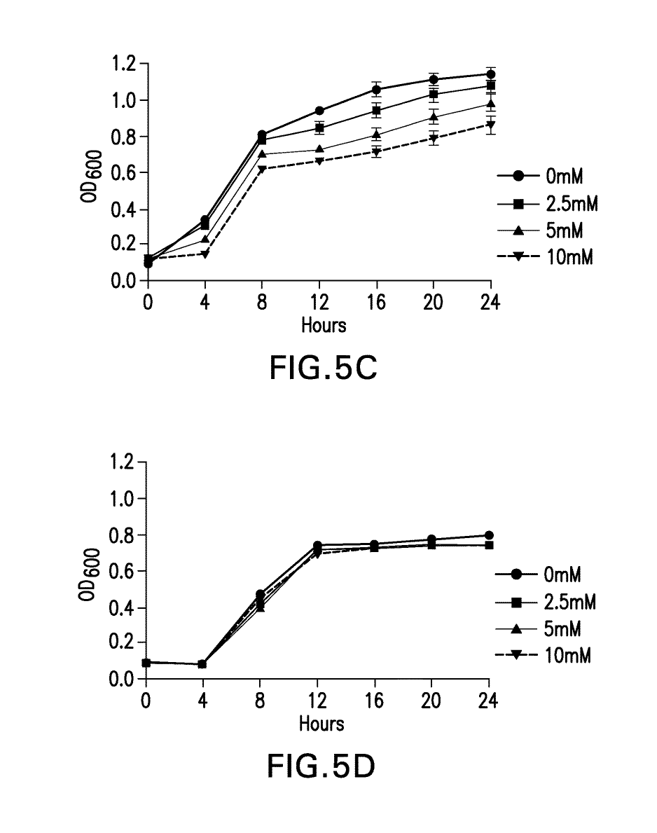

E. coli and MRSA strains were incubated at 37° C. with and without various concentrations of SNO-CAP-np (1, 2.5, 5, or 10 mg/mL) or captopril (2.5, 5, and 10 mg/mL) for 24 h. OD600 was plotted every 4 h, and background OD600 for SNO-CAP-np in TSB was subtracted. Each data set represents averages for 8 strains of either E. coli or MRSA, and conditions for each strain were measured in triplicate. For both species, all concentrations of SNO-CAP-np significantly inhibited bacterial growth compared to untreated controls in a dose-dependent manner for up to 24 h. Overall, E. coli was more sensitive than MRSA, and 10 mg/mL SNO-CAP-np lead to 100% growth reduction for both species (FIGS. 5A, 5B).

Based on theoretical calculation, the highest concentration of SNO-CAP-np (10 mg/mL) contained 2.76 mM captopril. Thus, captopril concentrations were titrated upwards (2.5, 5, or 10 mM) and likewise incubated with E. coli and MRSA. Interestingly, captopril showed an effect on E. coli growth in a dose dependent fashion (FIG. 5C), which was significant for all concentrations of captopril after 12 h. Captopril did not have an effect on the MRSA isolates tested (FIG. 5D).

Colony-Forming Units (CFU) Assay.

Following 24 h incubation of E. coli and MRSA, 10 μL aspirates from wells of the honeycomb plates were transferred to Eppendorf tubes with 990 μL PBS and gently vortexed. Controls were collected in the same fashion from wells containing only bacteria and TSB. The suspensions were serially diluted in PBS so that final concentrations were 10⁻⁶ of the incubated concentration, and 100 μL aliquots were plated on TSA plates for 24 h. As with the absorbance assays, 8 clinical strains each of MRSA and E. coli were evaluated (data represents an average for the 8 strains each of E. coli and MRSA), and all conditions were measured in triplicate. CFU's were counted and recorded. Percent survival was determined by comparing CFU counts of SNO-CAP-np- or captopril-treated bacteria to CFU counts of untreated bacteria. P-value <0.05 by unpaired t-test was considered significant.

SNO-CAP-Np are Bactericidal Against E. coli:

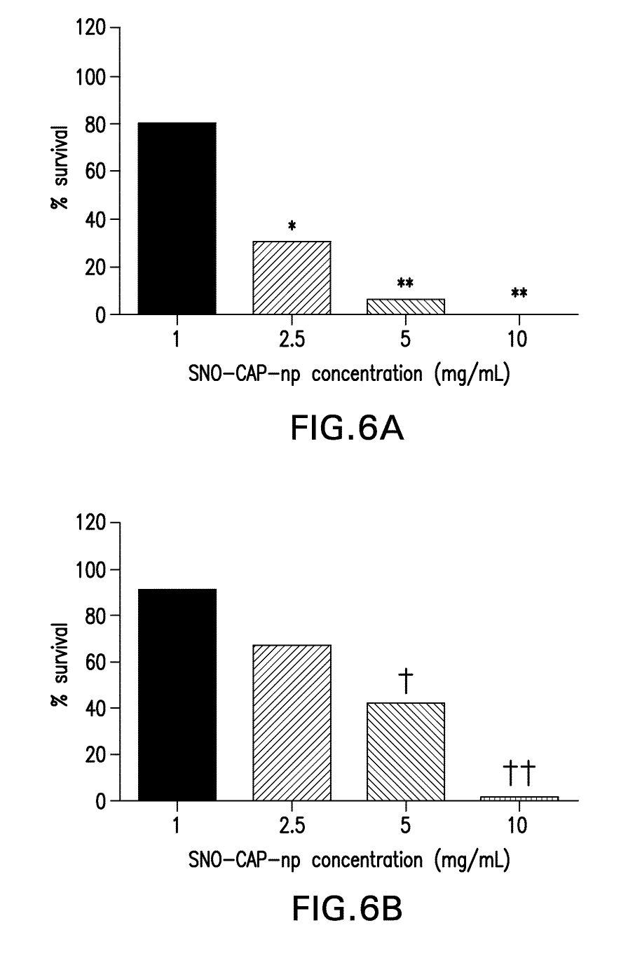

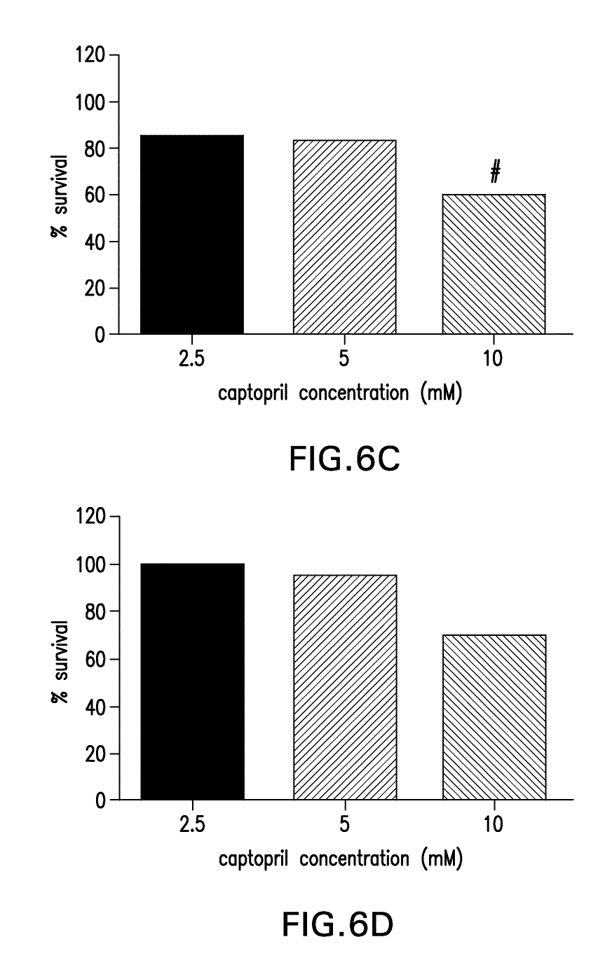

After incubation with either SNO-CAP-np or captopril, E. coli suspensions were diluted and plated on TSA, and CFU's were quantified after 24 h (FIGS. 6A, 6C). Average E. coli survival for 1, 2.5, 5 and 10 mg/mL SNO-CAP-np was 79.6, 30.2, 5.5 and 0.3% compared with untreated controls. Unpaired t-test analysis revealed that 2.5, 5 and 10 mg/mL SNO-CAP-np significantly inhibited E. coli growth (P=0.0007, <0.0001, <0.0001, respectively). The average E. coli survival for 2.5, 5 and 10 mM captopril was 85, 83.6 and 59.1% compared with untreated controls, and analysis via unpaired t-test revealed that only 10 mM captopril significantly inhibited E. coli growth (P=0.026).

SNO-CAP-Np are Bactericidal Against MRSA.

After incubation with either SNO-CAP-np or captopril, MRSA suspensions were diluted and plated on TSA, and CFU's were quantified after 24 h (FIGS. 6B, 6D). Average MRSA survival for 1, 2.5, 5 and 10 mg/mL SNO-CAP-np was 90.7, 67.1, 40.6 and 0.4% compared with untreated controls. Unpaired t-test analysis revealed that 5 and 10 mg/mL SNO-CAP-np significantly inhibited MRSA growth (P=0.02 and 0.0003, respectively). The average MRSA survival for 2.5, 5 and 10 mM captopril was 99.3, 95.6 and 69.5% compared with untreated controls. Unpaired t-test analysis revealed that none of these captopril concentrations significantly inhibited MRSA growth.

### 6.7 In Vivo Toxicity Assay of SNO-CAP Nanoparticles

In this example, zebrafish embryos (Danio rerio, wild type, 5D-Tropical strain) were obtained from Sinnhuber Aquatic Research Laboratory, Oregon State University, and exposures and evaluations were conducted according to Truong et al., 2011. Briefly, embryos were dechorionated at 6 hours post-fertilization (hpf) by Protease Type XIV (Sigma Aldrich). Control-np, Alexa 568-np, and SNO-CAP-np were each diluted to 0, 0.016, 0.08, 0.4, 2, 10, 50 and 250 ppm in fish water and vortexed. Each well of a 96-well plate was filled with 150 μL of a given dilution, in addition to one zebrafish embryo at 8 hpf (N=24 for each dilution). The plates were sealed with Parafilm and incubated at 26.5° C. on a 14 h light:10 h dark photoperiod.

Exposures were conducted over 5 days of development which encompasses gastrulation through organogenesis, the periods of development most conserved among vertebrates. All organ systems begin functioning during this time period and all of the molecular signaling pathways are active and necessary for normal development to occur. At 24 hpf, embryos were examined for mortality, developmental progression, notochord development, and spontaneous movement. At 120 hpf, the following larval morphology and behavioral endpoints were examined: body axis, eye, snout, jaw, otic vesicle, heart, brain, somite, pectoral fin, caudal fin, yolk sac, trunk, circulation, pigment, swim bladder, motility and tactile response. Effects were evaluated in binary notation as either present or not present. Untreated control and exposed groups were compared using Fisher's exact test for each endpoint, and p-value <0.05 for significance.

Results:

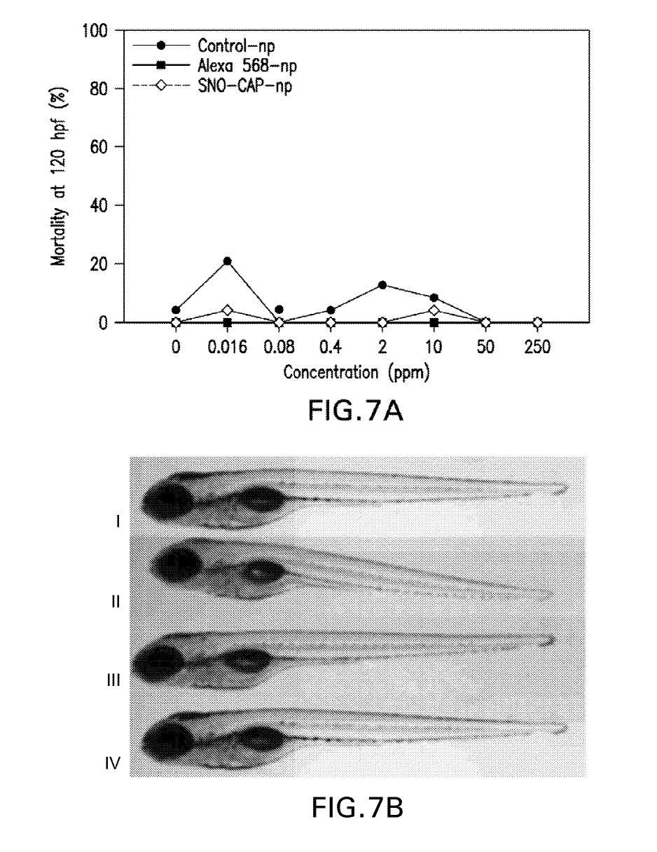

The results of the toxicity assay are shown at FIGS. 7A and 7B. The embryonic exposures did not elicit any toxic responses in the zebrafish after 5 days of exposure during a sensitive developmental time period. No nanoparticle treatments were significantly different from untreated controls with respect to mortality, morphology or behavior. Background mortality is maintained below 8.3% in the Harper Laboratory (Oregon State University), which is below the EPA ecological effects test guideline of 10%. Mortality did not differ between groups and was not significantly different than background for any exposure. There were no significant behavior abnormalities in the exposed zebrafish at 24 hpf or 120 hpf, as shown by normal patterns of spontaneous movement and standard touch responses.

The following examples (6.8-6.19) refer to the preparation, characterization, and efficacy of curcumin compositions and curcumin-encapsulated nanoparticles in accordance with one or more embodiments of the present application.

### 6.8 Preparation of Curcumin Composition and Synthesis of Curcumin Nanoparticles (curc-np)

In this example, a curcumin (Sigma-Aldrich, St. Louis, Mo., USA) stock solution was prepared at a concentration of 200 mg/mL in 100% of DMSO. For susceptibility testing, the stock was dilution in RPMI 1640 medium to a final concentration of 40 μg/mL. For aPI, the stock was diluted in PBS to concentrations of 1.0, 10 and 100 μg/mL. The final concentration of DMSO in both dilutions was less than 1%, such that the solvent did not contribute to observed fungicidal activity. A comparative concentration of curcumin incorporated in nanoparticles was used based on spectrophotometric release curves showing that each mg of curc-np contained 10 μg of curcumin. For susceptibility testing, 8 mg of curc-np was suspended in 1 mL of PBS and diluted in RPMI to a final concentration of 4.0 μg/mL (equivalent to 40 μg/mL of encapsulated curcumin) For aPI, 10 mg of curc-np was suspended in 1 mL of PBS and serially diluted to obtain 10 μg/mL, 100 μg/mL and 10 mg/L of curc-np (equivalent to 1.0, 10, and 100 μg/mL of encapsulated curcumin) The light source used was BLU-U® light model 4070 (DUSA pharmaceuticals, Wilmington, Mass., USA), which emits blue light at a wavelength of 417±5 nm. The doses used were 10 J/cm² (17 minutes), 20 J/cm² (34 minutes), and 40 J/cm² (68 minutes). BLU-U light was chosen as the light source due to its resonance with curcumin.

To create curc-np, tetramethyl orthosilicate (TMOS) was hydrolyzed by adding HCl, followed by sonication on ice. The mixture was refrigerated at 4° C. until monophasic. Curcumin was dissolved in methanol and combined with chitosan (4.4%), polyethylene glycol (4.4%) and TMOS-HCl (8.8%) to induce polymerization. The gel was lyophilized at ˜200 mTorr for 48-72 hours. The resulting powder was processed in a ball mill for ten 30-minute cycles to achieve smaller size and uniform distribution.

### 6.9 Antimicrobial Photodynamic Inhibition (aPI) with Curcumin and Curc-NP

For aPI optimization, fungal cells were submitted to different treatment conditions by varying the photosensitizer (PS) concentration and light dose, as described in Table 1, below. PS without photo activation and blue light alone served as dark toxicity and light controls, respectively. A 1% DMSO solution in control medium was evaluated for any contributing toxicity.

TABLE 1
Treatment Conditions for aPI Optimization

| | |
|---|---|
| Groups | Treatments |

Controls

| | |
|---|---|
| Untreated control (C) | T. rubrum microconidia only |
| Blue light (B.L.) | T. rubrum microconidia irradiated with |
| | blue light 417 ± 5 nm. |
| curcumin 10 μg/mL, | T. rubrum microconidia treated with curcumin 10 |
| | μg/mL for 10 minutes under light protection. |
| curcumin 1.0 μg/mL, | T. rubrum microconidia treated with curcumin 1.0 |
| | μg/mL for 10 minutes under light protection. |
| curcumin 0.1 μg/mL, | T. rubrum microconidia treated with curcumin 0.1 |
| | μg/mL for 10 minutes under light protection. |
| curc-np 10 μg/mL, | T. rubrum microconidia treated with curc-np 10 |
| | μg/mL for 10 minutes under light protection. |
| curc-np 1.0 μg/mL, | T. rubrum microconidia treated with curc-np 1.0 |
| | μg/mL for 10 minutes under light protection. |
| curc-np 0.1 μg/mL, | T. rubrum microconidia treated with curc-np 0.1 |
| | μg/mL for 10 minutes under light protection. |
| Blue light 40 J/cm² | T. rubrum microconidia irradiated with blue light |
| | dose of 40 J/cm² |
| Blue light 20 J/cm² | T. rubrum microconidia irradiated with blue light |
| | dose of 20 J/cm² |
| Blue light 10 J/cm² | T. rubrum microconidia irradiated with blue light |
| | dose of 10 J/cm² |

Treatments

| | |
|---|---|
| curcumin + Blue light | T. rubrum microconidia treated with curcumin 10 |
| 40 J/cm | mg/L, for 10 minutes under light protection, |
| | followed by irradiation with blue light |
| | dose of 40 J/cm². |
| curcumin + Blue light | T. rubrum microconidia treated with curcumin 10 |
| 20 J/cm² | mg/L, for 10 minutes under light protection, |
| | followed by irradiation with blue light |
| | dose of 20 J/cm². |
| curcumin + Blue light | T. rubrum microconidia treated with curcumin 10 |
| 10 J/cm² | μg/mL for 10 minutes under light protection, |
| | followed by irradiation with blue light dose of 10 |
| | J/cm². |
| curc-np + Blue light | T. rubrum microconidia treated with curc-np 10 |
| 40 J/cm | μg/mL for 10 minutes under light protection, |
| | followed by irradiation with blue light dose of 40 |
| | J/cm². |
| curc-np + Blue light | T. rubrum microconidia treated with curc-np 10 |
| 20 J/cm² | μg/mL for 10 minutes under light protection, |
| | followed by irradiation with blue light dose of 20 |
| | J/cm². |
| curc-np + Blue light | T. rubrum microconidia treated with curc-np 10 |
| 10 J/cm² | mg/L, for 10 minutes under light protection, |
| | followed by irradiation with blue light |
| | dose of 10 J/cm . |

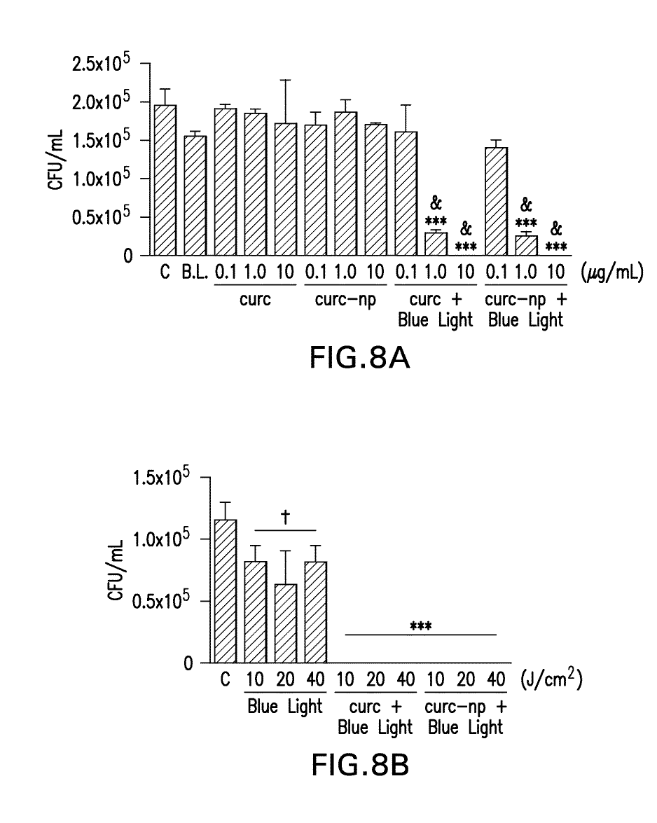

A range of curcumin concentrations and blue light doses were evaluated (Table 1). At a light dose of 40 J/cm², concentrations of 1.0 and 10 μg/mL of curcumin (cure) and curc-np significantly decreased fungal viability in a dose dependent manner compared to untreated control (p<0.0001), with the highest concentration achieving complete growth inhibition (FIG. 8a). The lowest PS concentration (0.1 μg/mL) did not differ significantly from untreated control. PS without photoactivation did not reduce fungal burden at the three concentrations tested (p<0.05), nor did the 1% DMSO solution (data not depicted). In combination with the most effective PS concentration, all three blue light doses completely inhibited T. rubrum growth (p<0.0001, FIG. 8b) and were significant compared to untreated and blue light controls. Blue light alone, without the addition of PS, exhibited fungicidal activity (p<0.05), but did not completely inhibit growth, with no differences observed between light fluences. Based on these results, a PS concentration of 10 μg/mL and a blue light dose of 10 J/cm² were chosen for all subsequent analyses.

### 6.10 Susceptibility Testing and aPI Growth Curve for Curcumin and Curc-NP

Susceptibility of T. rubrum to ground-state curcumin was tested by a microdilution method according to CLSI M38-A.49, 52 Itraconazole concentration ranged from 0.015 μg/mL to 8 μg/mL and curcumin and curc-np concentrations from 0.0012 μg/mL to 20 μg/mL. A 1% DMSO solution in control medium was evaluated. The MIC value was defined as the concentration required for 80% fungal growth compared to untreated control.12, 49 Growth kinetics of ground-state curcumin compared to aPI was also evaluated. Growth was evaluated for 7 days at 28° C. using a Bioscreen C growth curve system (Growth Curves USA, Piscataway, N.J., USA).

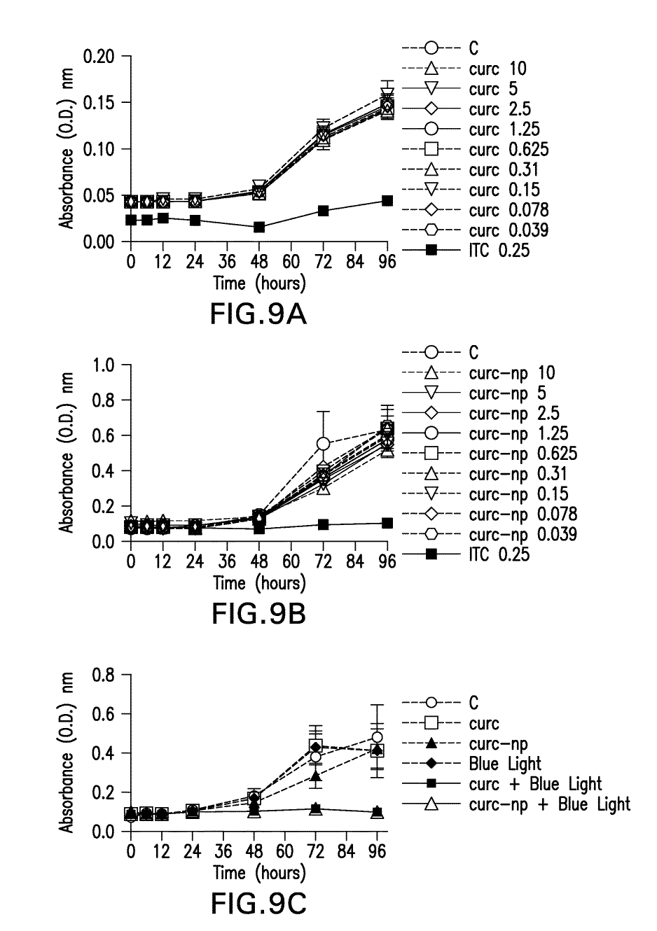

The intrinsic antifungal activity of ground-state curcumin was evaluated by incubating T. rubrum with a range of curc and curc-np concentrations (FIGS. 9A, 9B). Seven-day incubation with ground-state curcumin did not yield significant 80% reduction of fungal growth. A 1% DMSO solution did not exert any fungicidal activity (data not represented). Itraconazole was used as a comparative control to test the virulence of the clinical T. rubrum strain. The MIC value of itraconazole was 0.25 μg/mL, which is within the reported range. Differences in growth kinetics between T. rubrum treated with ground-state and photoactivated curcumin was observed at 48 hours of incubation (FIG. 9C). A steady increase of growth was observed for the PS control, while aPI completely inhibited growth for the full seven days (represented until 96 hours).

### 6.11 Measurement of Reactive Oxygen Species (ROS) and Reactive Nitrogen Species (RNS) for Curcumin and Curc-NP

Intracellular generation of ROS and RNS was evaluated using 50 μM of 2′,7′dichlorodihydrofluorescein diacetate (H₂DCFDA, Invitrogen) to quantify ROS, 10 μM of 4-amino-5-methylamino-2′,7′-difluorofluorescein (DAF-FM, Invitrogen) to quantify NO., and 50 μM dihydrorhodamine 123 (DHR 123, Invitrogen) to quantify ONOO.. Following aPI, samples were incubated with fluorescent probes for 30 minutes at 28° C., and subsequently analyzed with flow cytometry (Becton Dickinson™ LSRII, USA) using a 530/30 nm band pass filter for fluorescence detection. The Mean Fluorescence Intensity (MFI) was considered to determine radical production. Data analyses were performed using FlowJo 10.1 software.

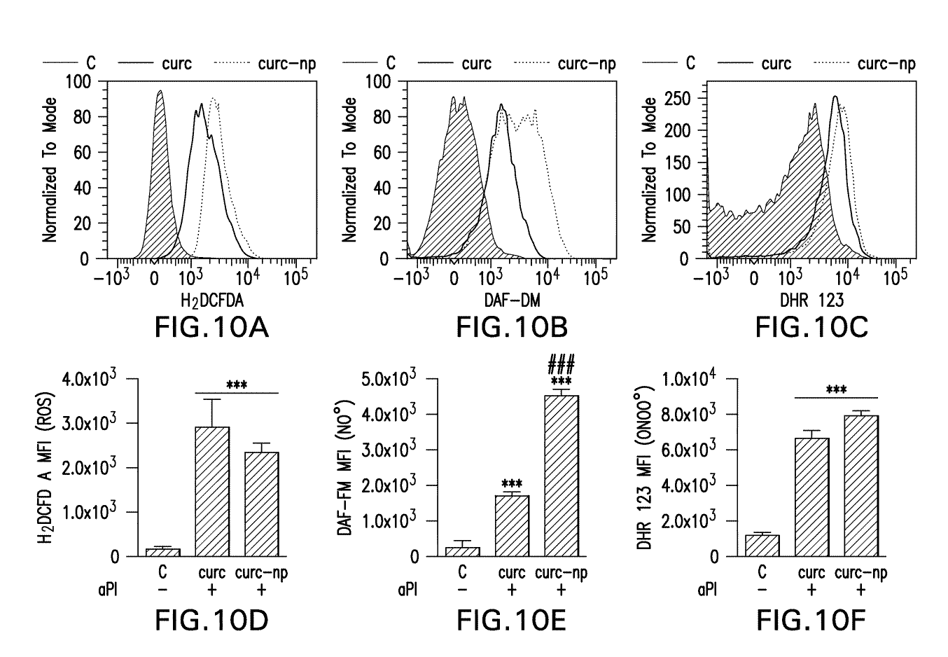

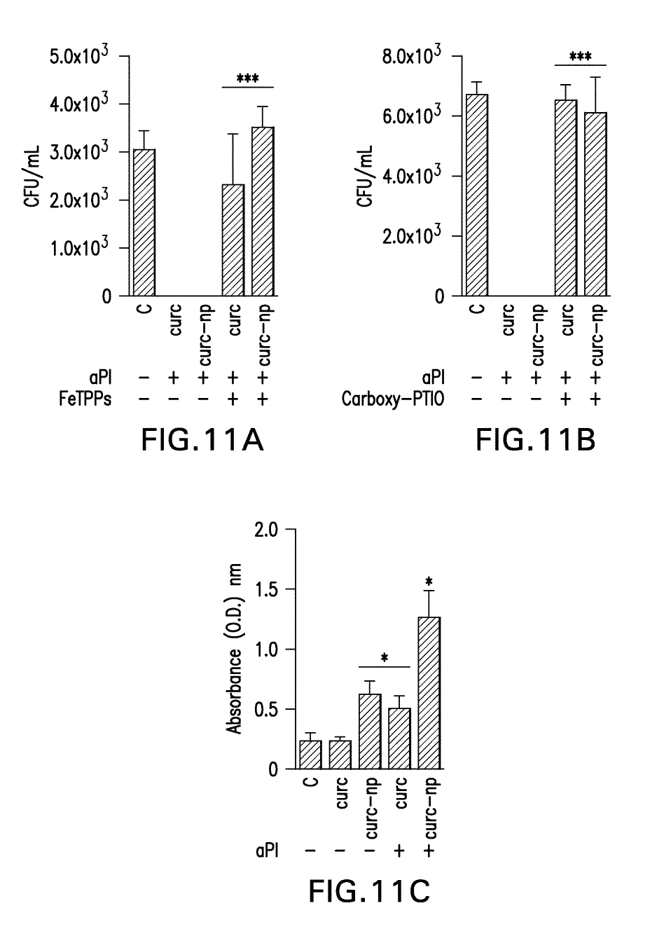

Compared to untreated control, photoactivated curcumin induced a significant increase in the generation of both species (p<0.0001, FIGS. 10A-F). Treatment with curc and curc-np induced a fold-change in ROS production by 17 and 13, respectively (FIGS. 10A, 10D). For NO. production, a greater disparity between curc and curc-np was observed, with a fold change of 6 and 16, respectively (FIGS. 10B, 10E). Measurement of ONOO. production demonstrated the smallest fold-change of 7 and 6 (FIGS. 10C, 10F).

### 6.12 Treatment with ROS and RNS Scavengers

Different ROS and RNS scavengers were used to evaluate the effect of radical stress inhibition on aPI efficacy. The scavengers included: 5,10,15,20-tetrakis-(4-sulfonatophenyl)-porphyrinato iron (III) chloride (FeTPPs) (1 and 0.1 mM, Calbiochem) as a ONOO. scavenger, 4,5-dihydroxy-1,3-benzenedisulfonic acid disodiumsalt hydrate (Tiron) (1.0 and 10 mM, Sigma-Aldrich, St. Louis, Mo., USA) as a O₂.⁻ scavenger, sodium pyruvate (0.1, 1.0 and 10 mM, Sigma-Aldrich, St. Louis, Mo., USA) as a hydrogen peroxide scavenger, carboxy-PTIO (2.0 and 0.2 mM, Cayman chemical, Ann Arbor, Mich., USA) as a NO. scavenger, D-mannitol (100 mM, Sigma-Aldrich, St. Louis, Mo., USA) as a hydroxyl radical scavenger and sodium azide (1.0, 10 mM and 1.0 M, Sigma-Aldrich, St. Louis, Mo., USA) as an ¹O₂ scavenger. Scavengers were added to fungal suspensions immediately before initiation of aPI and incubated for 1 h with RPMI 1640 without phenol red plus 2% glucose at 28° C. To evaluate fungal viability, 150 μL of the fungal suspensions were plated onto PDA, and incubated at 28° C. for 72 hours. The HT TitierTACS™ assay kit (Trevigen, Gaithersburg, Md., USA) was used to evaluate the occurrence of apoptosis after aPI.

The results showed that none of the concentrations of Tiron (superoxide anion scavenger), sodium pyruvate (hydrogen peroxide scavenger), D-mannitol (hydroxyl radical scavenger) or sodium azide (singlet oxygen) inhibited aPI efficacy. Interestingly, T. rubrum growth was relatively intact despite aPI only in the presence of RNS scavengers, particularly FeTPPs (ONOO. scavenger) and carboxy-PTIO (NO. scavenger) (FIGS. 11A and 11B). The apoptosis assay showed that curc alone did not induce apoptosis of T. rubrum cells compared to untreated control; however, after irradiation with blue light, there was a significant trend towards increased apoptosis (p<0.05, FIG. 11C). Curc-np, on the other hand, significantly increased the occurrence of apoptosis in comparison to untreated control (p<0.05). Additionally, an extreme augmentation of apoptotic fungal cells was observed after treatment with curc-np in combination with blue light (p<0.0001).

### 6.13 Phagocytosis Assay

Macrophages were challenged with T. rubrum and treated with aPI to investigate the efficacy against infected mammalian cells. Specifically, J774.16 macrophages were grown at 37° C. with 10% CO₂ in DMEM (Cellgro, Manassas, Va., USA). The fungal-macrophage cell proportion was 1:1, with 5.0×105 fungal cells to 5.0×105 macrophage cells. After challenging macrophages with T. rubrum microconidia, the cells were submitted to aPI, followed by incubation in the 10% CO2 chamber at 37° C. for 24 hours. The macrophages were lysed with cold distilled water and the lysate plated onto PDA and incubated at 28° C. for 72 hours.

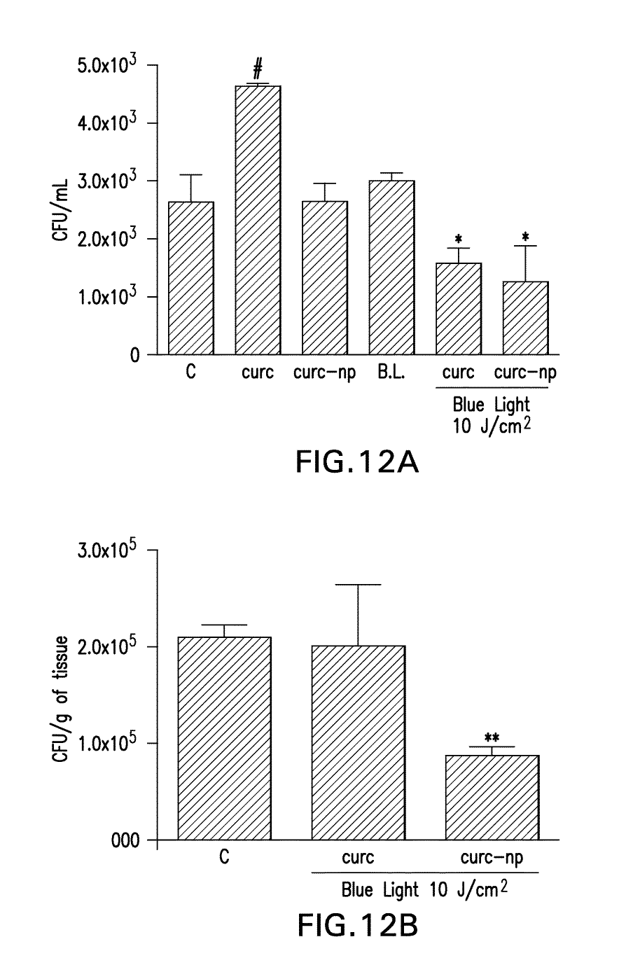

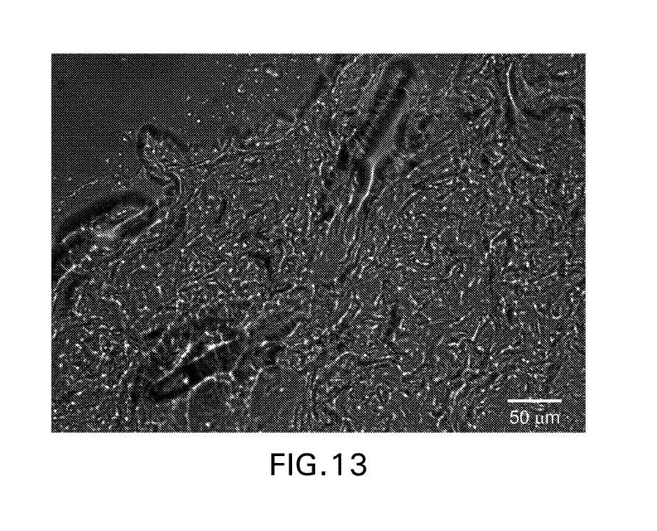

aPI with cure or curc-np significantly reduced fungal burden compared to untreated, dark toxicity and blue light controls (p<0.05, FIG. 12). Interestingly, ground-curcumin in the absence of aPI caused a decrease in macrophage-induced destruction of T. rubrum cells (p<0.05).

### 6.14 In Vivo Antimicrobial Photodynamic Therapy (aPDT) Pilot Study

BALB/c mice were subcutaneously infected with T. rubrum. Seven days post-infection, mice (n=2 per group) were submitted to one treatment of aPDT, using 500 μg/mL of curcumin and curc-np suspended in coconut oil and 10 J/cm² of blue light. Pre-irradiation incubation time was 30 minutes, under light protection. Tissue was homogenized and CFUs quantified three days post treatment.

The result of the pilot in vivo study revealed significant differences between the two curcumin formulations following a single treatment. While the cure group did not significantly reduce fungal burden compared to untreated control, the curc-np group demonstrated statistically significant reduction in fungal cell survival (58.3%) compared with untreated control and cure groups (p<0.0001).

The following examples (Sections 6.15-6.19) refer to the preparation, characterization, cytotoxicity, and efficacy of curcumin-encapsulated hybrid hydrogel nanoparticles in accordance with one or more embodiments of the present application.

### 6.15 Synthesis of Curcumin Hybrid Hydrogel Nanoparticles

To create the curcumin hybrid hydrogel nanoparticles, first Tetramethyl orthosilicate (TMOS) was hydrolyzed by adding HCl, followed by 20-minute sonication in ice water bath. Curcumin was dissolved in methanol and combined with chitosan (4.4%) (buffer), polyethylene glycol (4.4%), and then vortexed. The vortex mixture was then combined with the hydrolyzed TMOS (TMOS-HCl [8.8%]) to induce polymerization. The resulting gel was lyophilized at ˜200 mTorr for 48-72 hours, removing all traces of methanol. The resulting powder was processed in a ball mill for ten 30-minute cycles to achieve smaller size and uniform distribution. Results were consistently reproducible. Control nanoparticles were synthesized identically to curcumin hydrogel nanoparticles, without the addition of curcumin

Clinical Isolates:

clinical isolates were collected from patients' wounds at Montefiore Medical Center (Bronx, N.Y.). Twelve clinical isolates were evaluated, including 8 MRSA and 4 P. aeruginosa strains, and stored at 4° C. on tryptic soy agar (TSA).

### 6.16 Characterization of Curcumin Hybrid Hydrogel Nanoparticles

Scanning Electron Microscopy:

the nanoparticles were plated on poly-L-lysine-coated coverslips, critical point dried using liquid CO₂ in Samdri-795 Critical Point Dryer (Tousimis, Rockville, Md.), and sputter coated with chromium in Q150T ES Sputter Coater (Quorum Technologies Ltd, East Sussex, UK). Samples were examined under Supra Field Emission Scanning Electron Microscope (Carl Zeiss Microscopy, Peabody, M A) with 3 kV accelerating voltage.

Dynamic Light Scattering:

A suspension of curcumin hybrid hydrogel nanoparticles (1 mg/ml) was sonicated in distilled water, and size was measured using DynaPro NanoStar (Wyatt Technology, Santa Barbara, Calif.). Experiments were conducted in triplicate, with 40 acquisition attempts (acquisition length 5 seconds) per sample. Average nanoparticle hydrodynamic diameter and polydispersity index were calculated from results.

In Vitro Release Kinetics:

The amount of encapsulated curcumin was evaluated by comparing spectrophotometric absorbance of the curcumin hydrogel nanoparticles dissolved in methanol to a standard curve of curcumin using Lambda 2 UV/VIS spectrometer (PerkinElmer, Waltham, Mass.). Release over time was evaluated by dispersing individual aliquots of 2 mg/ml curc-np (n=4 per time point) in phosphate buffered saline (PBS, pH=7.4) and incubating at 37° C. under at 100 rpm using innova 2300 platform shaker (New Brunswick Scientific, Enfield, C T). At 2-hour intervals, individual samples were pelleted and dissolved in methanol to solubilize unreleased curcumin. The amount released was calculated by dividing the absorbance at each time point by the absorbance of the estimated encapsulated maximum.

Results:

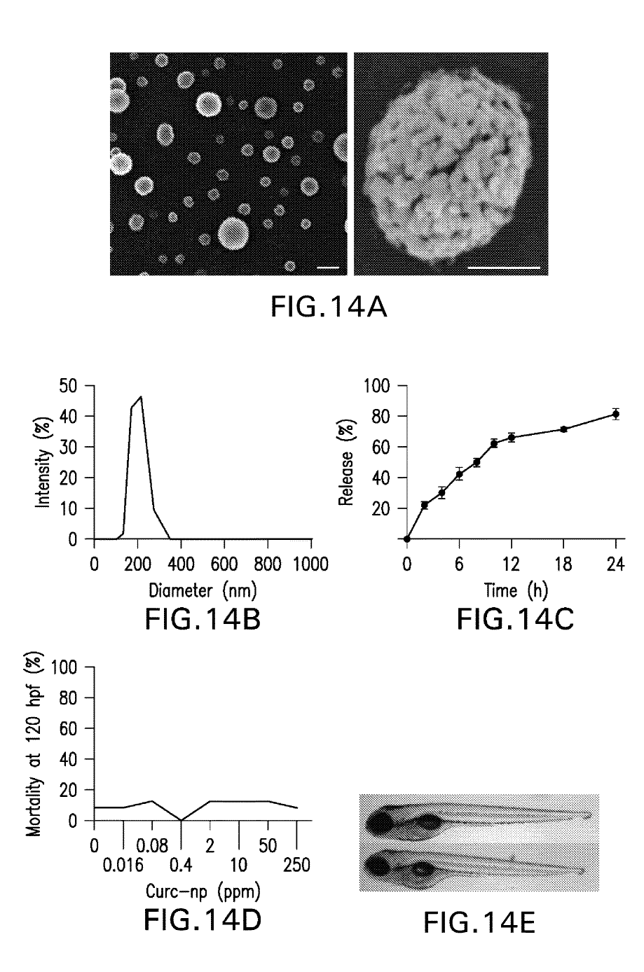

Scanning electron microscopy revealed distinct spherical nanoparticles with irregular surface structure indicative of the porous matrix lattice (FIG. 14A). Dynamic light scattering showed a narrow size range with average hydrodynamic diameter of 222±14 nm (FIG. 14B), likely an overestimate as nanoparticles swell with moisture. The total theoretical amount of encapsulated curcumin per mg of particle was calculated to be 10 ug. Release occurred in a controlled and sustained manner, with incomplete release of the calculated maximum after 24 hours (FIG. 14C). In the first 6 hours, 42.3% of curcumin was released, increasing to 81.5% after 24 hours, amounting to a total release of 8.15 μg per mg of particle (e.g., 1 mg/ml curc-np=8.15 μg/ml curcumin) Our results therefore indicate that complete release of encapsulated curcumin does not occur, and the therapeutic efficacy observed throughout this study occurred at concentrations less than the calculated theoretical maximum doses.

### 6.17 Cytotoxicity of Curcumin Hybrid Hydrogel Nanoparticles

Cellular cytotoxicity assay: Using the semiquantitative FDA (fluorescein diacetate) metabolic assay, the susceptibility of murine PAM212 keratinocytes to curcumin hydrogel nanoparticle was assessed. 2×10⁴ keratinocytes were plated in a 96-well plate and grown overnight in Dulbecco's modified Eagle's medium (DMEM) containing 10% fetal bovine serum (FBS), 1% HEPES, 1% nonessential amino acids, and 1% penicillin-streptomycin. Cells were incubated with 200 ul of media containing curcumin hydrogel nanoparticles 5 mg/ml for 24 hours at 37° C., 5% CO₂. Metabolic activity was measured by FDA assay and statistical analysis conducted using Student's t-test.

Zebrafish Cytotoxicity Assay:

Zebrafish embryos (Danio rerio, wild type, 5D-Tropical strain) were obtained. Curcumin hydrogel nanoparticles were dispersed in fish water at stock concentration of 1000 ppm prior to serial dilutions. Embryos were dechorionated at six hours post-fertilization (hpf) by pronase enzyme degradation and at eight hpf were transferred to 96-well plates, one embryo per well (n=24). Plates were incubated at 26.5° C. under a photoperiod of 14:10 hour light:dark cycle. Effects were evaluated in binary notation as either present or not present. Statistical analysis was performed using Fisher's exact test at p<0.05 for each endpoint.

Results:

The effect of curcumin hydrogel nanoparticles on viability of PAM212 keratinocytes was measured by FDA assay. Cells treated with curcumin hydrogel nanoparticles

5 mg/ml exhibited 81.7% cell viability as compared to untreated control (p≦0.005, data not shown). In vivo toxicological impact of curcumin hydrogel nanoparticles was assessed via embryonic zebrafish assay (FIGS. 14D, 14E). At 24 hours post-fertilization, embryos were examined for mortality, developmental progression, notochord development, and spontaneous movement. At 120 hpf, larval morphology and behavior were examined. Body axis, eye, snout, jaw, otic vesicle, heart, brain, somite, pectoral fin, caudal fin, yolk sac, trunk, circulation, pigment, and swim bladder malformations were recorded, as well as motility and tactile response. Exposure to curcumin hydrogel nanoparticles did not elicit any toxic responses after 5 days of exposure during a sensitive developmental time period. No statistical differences were appreciated from fish water control with respect to mortality, development, larval morphology, or behavioral endpoints.

### 6.18 Efficacy of Curcumin Hybrid Hydrogel Nanoparticles

Susceptibility of Bacterial Strains to Curcumin Hydrogel Nanoparticles:

For each bacterial strain, 1 μl aliquots of known bacterial suspension were transferred to 100-well honeycomb plates with 199 μl TSB, containing 2.5, 5, and 10 mg/ml of curcumin hydrogel nanoparticles and control nanoparticles. Background absorbance of each concentration was accounted for by wells containing nanoparticles and TSB alone. Optical density readings were acquired at 600 nm hourly for 24 hours using a microplate reader (Bioscreen C, Growth Curves USA, Piscataway, N.J.). Statistical significance of growth was assessed by 2-way ANOVA.

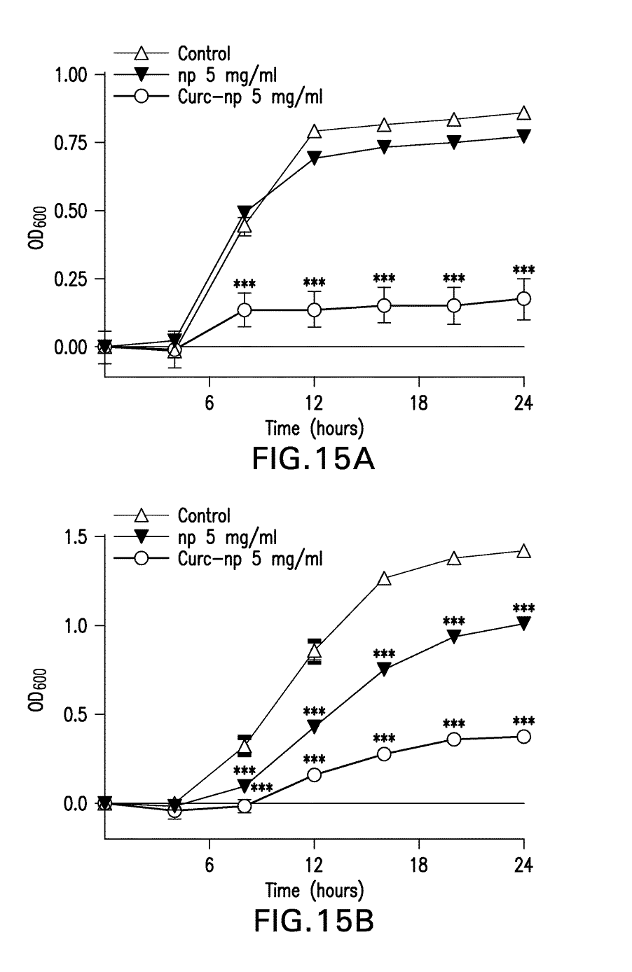

Based on curcumin hydrogel nanoparticles release kinetics, treatment with 5 mg/ml of curcumin hydrogel nanoparticles corresponded to approximately 40.75 μg/ml of curcumin released over 24 hours. For MRSA (FIG. 15A), curcumin hydrogel nanoparticles exhibited a significant antimicrobial effect from t=8 hours onward in comparison to both untreated control and control nanoparticles (np) (p≦0.0001). Control np did not exhibit any significant activity compared to untreated control (p>0.05). For P. aeruginosa (FIG. 15B), curcumin hybrid hydrogel nanoparticles exhibited a significant effect against control np (p≦0.05) and untreated control (p≦0.0001) from t=8 hours onward. Control np exhibited a significant effect compared to untreated control from t=8 hours onward (p≦0.0001), attributable to the physical presence of particles. The growth inhibition exhibited by control nanoparticles is consistent with prior studies conducted using this technology and can be attributed to the physical presence of particles, which interferes with cell-cell interactions, and intrinsic properties of nanoparticle components, e.g., chitosan. However, the significantly greater activity of curcumin hydrogel nanoparticles as compared to control np highlights curcumin's independent antimicrobial effects, notably more active against MRSA compared to P. aeruginosa.

Transmission Electron Microscopy (Mode of Action of Curcumin):

A suspension of 5×10⁸ MRSA cells was incubated for 6 and 24 hours with and without 5 mg/ml of control nanoparticles and curcumin hydrogel nanoparticles. Samples were fixed with 4.0% paraformaldehyde and 5% glutaraldehyde in 0.2 M sodium cacodylate buffer mixed 1:1 with serum free media, enrobed in 3% gelatin, postfixed with 1% osmium tetroxide followed by 1% uranyl acetate, dehydrated through a graded series of ethanol and embedded in Spurrs resin (Electron Microscopy Sciences, Hatfield, Pa.). Ultrathin sections were cut on Reichert Ultracut UCT, stained with uranyl acetate followed by lead citrate and viewed on 1200EX transmission electron microscope (JEOL, Peabody, M A) at 80 kV in order to explore the mode of action of curcumin hybrid hydrogel nanoparticles' antimicrobial activity.

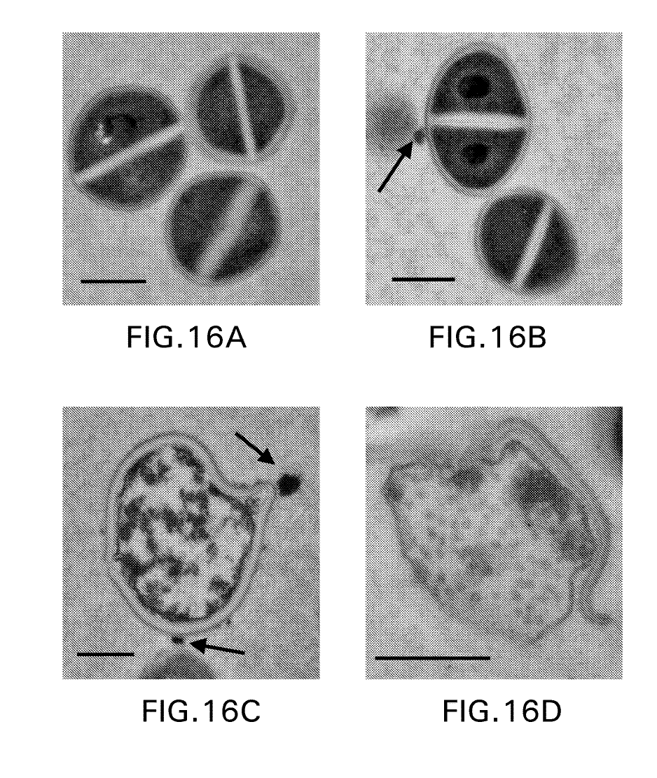

Untreated MRSA (FIG. 16A) showed intact cellular architecture with uniform cytoplasmic density and highly contrasting cross wall. After 24 hours, MRSA incubated with control np did not exhibit changes in cellular architecture compared to untreated control despite visible interaction with nanoparticles (FIG. 16B). In contrast, 6 hours after treatment with curc-np (FIG. 16C), MRSA cells displayed cellular edema and distortion in association with particles contacting the cell wall, with subsequent lysis and extrusion of cellular contents after 24 hours (FIG. 16D).

In Vivo Infected Murine Burn Model:

Dorsal hair of Balb/c mice (6-8 weeks; National Cancer Institute, Frederick, Md.) was shaved, and full-thickness 5-mm diameter burn injuries were created by applying a calibrated 160° C. heated bar to the backs for 10 seconds (n=10 wounds per group). A suspension of MRSA containing 5×10⁸ cells was inoculated onto each wound. Treatments were commenced 24 hours after infection. Wound tissue was excised on days 3 and 7, homogenized in 10 ml of PBS, and plated onto TSA. CFUs were quantified and analyzed for statistical significance using Student's t-test.

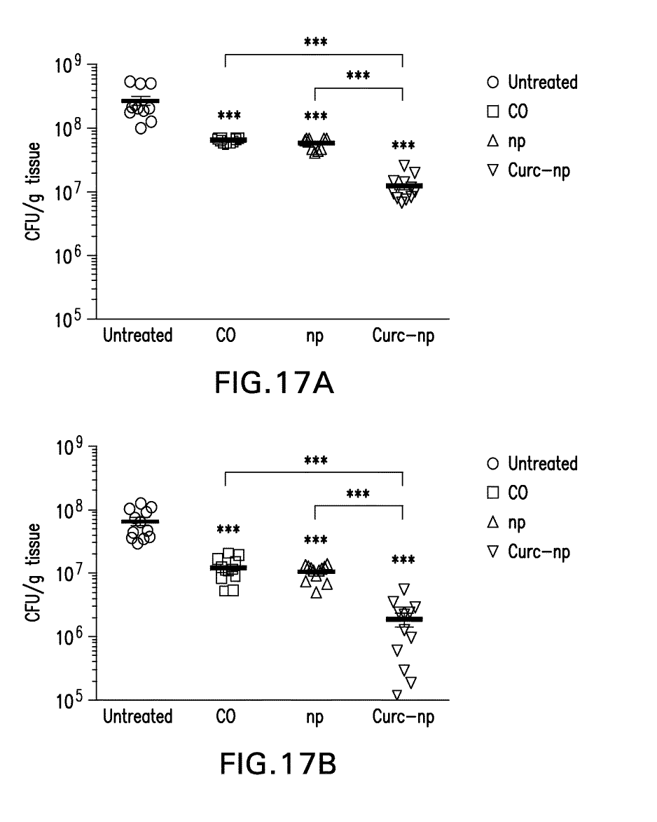

Wounds treated with curcumin hydrogel nanoparticles showed statistically significant reductions in bacterial counts on both days 3 (FIG. 17A) and 7 (FIG. 17B) compared to untreated infected control, coconut oil (delivery vehicle control), and control np wounds (p≦0.0001). Independent antimicrobial effects were exerted by coconut oil, as shown previously, but were significantly enhanced by addition of curcumin hybrid hydrogel nanoparticles.

In Vivo Murine Burn Model:

Burn wounds were created on Balb/c mice as detailed above (n=10 wounds per group) and treatment administered daily. Coconut oil was used as delivery vehicle for all treatment groups except silver sulfadiazine, and was evaluated independently. Daily photographs were taken and change in wound area relative to initial area was calculated using ImageJ software (National Institutes of Health, Bethesda, Md.), with statistical significance determined by 2-way ANOVA. On day 13, wounds were excised, fixed in 10% formalin, and embedded in paraffin. Four-micron vertical sections were stained with hematoxylin and eosin (H&E), Masson's trichrome, and CD34 to observe morphology, collagen deposition and angiogenesis (microvessels), respectively. Slides were observed under light microscopy and images were captured without further processing. Slides were numbered without indication of cohort to blind interpretation. Collagen deposition was measured by intensity using ImageJ. Ten HPFs (40×) were evaluated per section and analyzed for statistical significance using Student's t-test.

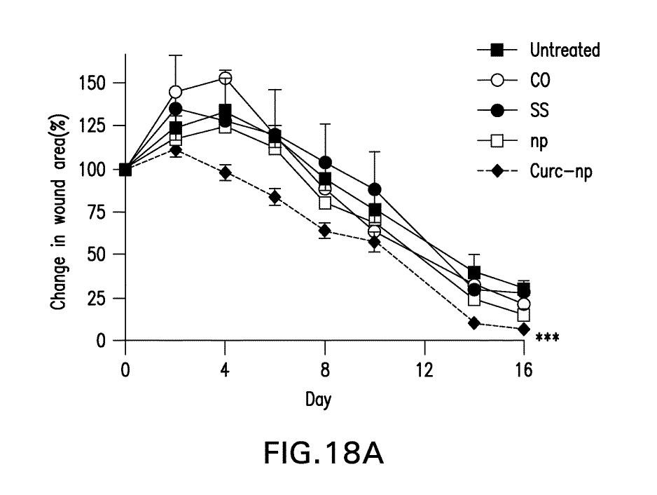

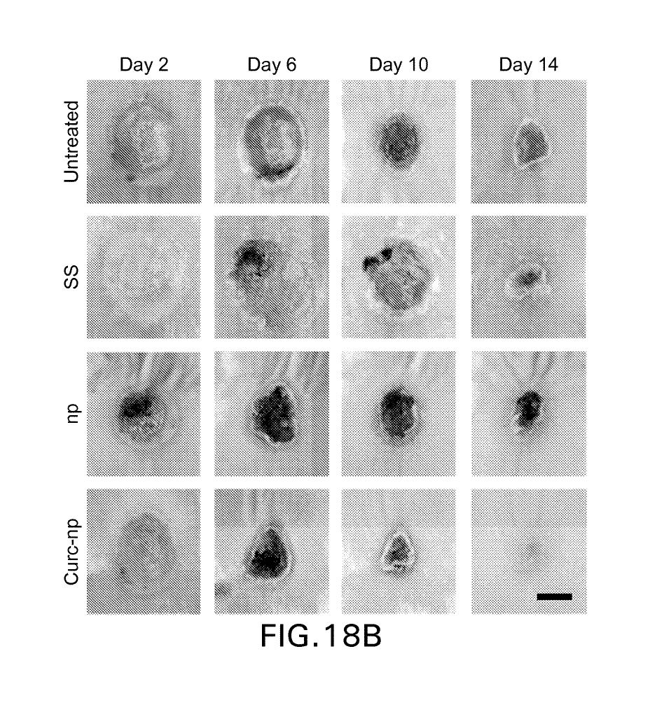

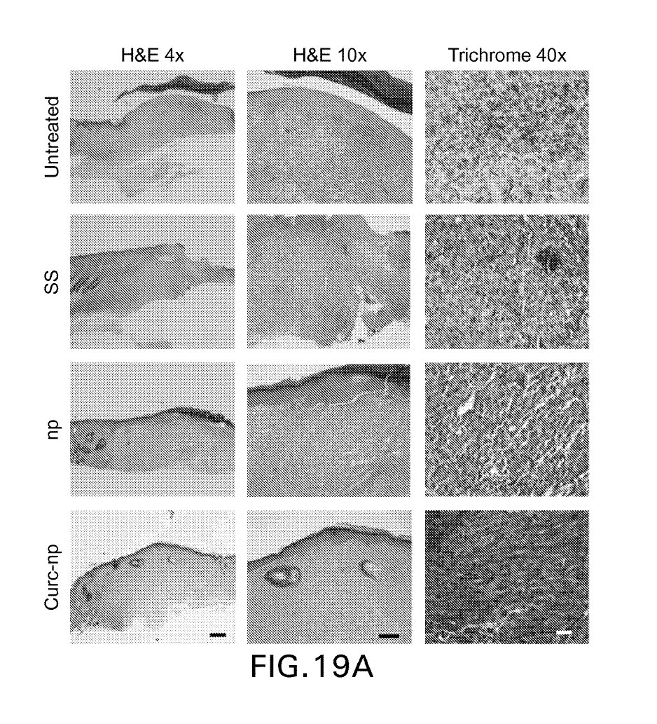

Topical administration of curcumin hybrid hydrogel nanoparticles significantly accelerated wound healing in mice as compared to untreated control, coconut oil, control np, and silver sulfadiazine groups (p≦0.0001, FIG. 18). Burn wounds demonstrate an expanding zone of inflammation in early stages post-injury, corresponding to progressive tissue loss. Curcumin hydrogel nanoparticles mitigated the observed wound expansion, and on day 4 curcumin hydrogel nanoparticle-treated wounds measured 98.1±4.4% compared to day 0, in contrast to size increases in untreated control (132.9±4.3%), coconut oil (153.0±4.04%), control np (124.7±4.41%), and silver sulfadiazine (127.5±13.2%). In addition to accelerated closure, qualitative assessment demonstrated that wounds treated with curcumin hybrid hydrogel nanoparticles displayed more well-formed granulation tissue and re-epithelialized earlier than other groups.

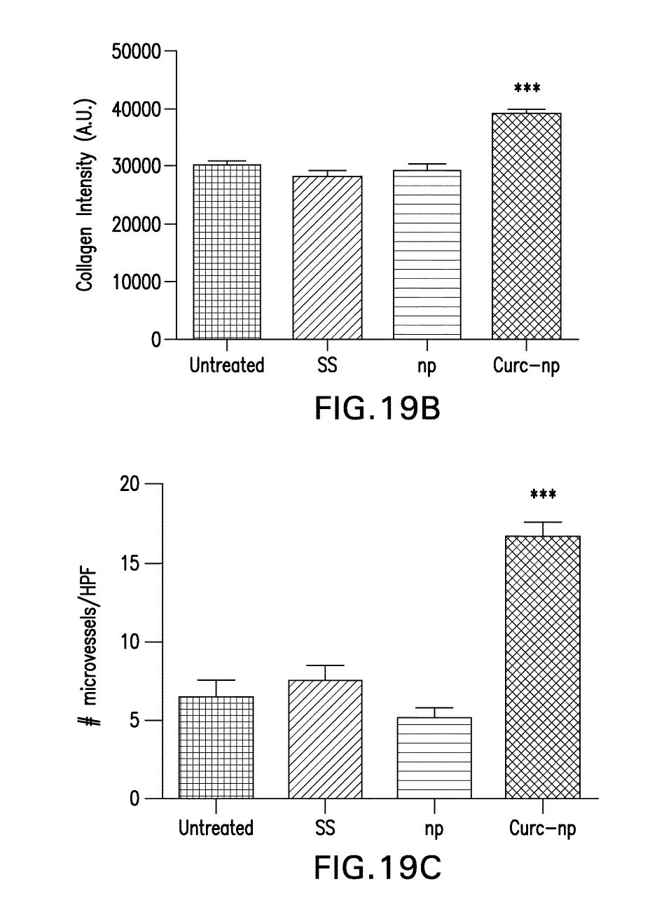

Further, histologic evaluation of wound sections from day 13 revealed distinct differences in maturity of the epidermis/dermis and quality of granulation tissue between curcumin hydrogel nanoparticles and other groups (FIG. 19A). While curcumin hybrid hydrogel nanoparticles demonstrated accelerated maturation and a well formed epidermis with compact orthokeratosis, other groups displayed inflammatory granulation tissue and partially re-epithelialized epidermis with overlying serum crust.

Evaluating collagen deposition, untreated control, silver sulfadiazine, and control np wounds displayed pale, necrotic, haphazardly deposited immature collagen (FIG. 19A). In contrast, the curcumin hydrogel nanoparticle-treated wounds displayed well organized compact collagen bundles, which were oriented parallel to the epidermis. Masson's trichrome staining revealed significantly increased collagen intensity (in arbitrary units, A.U.) in curcumin hydrogel nanoparticle-treated wounds compared to all other wounds (p≦0.0001; FIG. 19B).

New vessel formation, a hallmark of the proliferative phase of healing, was evaluated using CD34 staining. There was significantly greater neovascularization in wounds treated with curcumin hydrogel nanoparticles compared to all other groups (p≦0.0001; FIG. 19C), determined by number of stained microvessels per high-power field (HPF; 40×; 10 fields).

In Vitro Keratinocyte Migration Assay:

To explore a potential mechanism of curc-np in wound healing, a keratinocyte cellular migration assay was performed. Murine PAM212 keratinocytes were seeded in 6-well plates and grown until confluent. Four scratches were applied per well using 200 μl pipette tips prior to incubation with and without 0.5 mg/ml curc-np. Cell migration over 24 hours was imaged by time-lapse microscopy at 2-hour intervals in an environmental chamber using 4D spinning-disk confocal microscope (PerkinElmer, Waltham, Mass.) with 10× objective and Orca ER digital camera (Hamamatsu, Bridgewater, N.J.). Statistical analysis was conducted using Student's t-test.

No significant difference in relative wound area or migration rate was observed between untreated, control np and curcumin hydrogel nanoparticle-treated keratinocytes at 12 or 24 hours post-administration of scratch to cell monolayer (data not shown).

### 6.19 Example Formulations and Efficacy of Curcumin Hybrid Hydrogel Nanoparticles

Curcumin Containing Coconut Oil:

High purity curcumin is dissolved in melted coconut oil (up to several grams of curcumin per ten mls of melted coconut oil). The well mixed solution is then cooled. The solid material can be applied directly to the skin. The coconut oil melts at body temperature insuring ease of delivery. The blocks of curcumin containing coconut oil can be prepared as a roll on tube to be applied to targeted sites.

Curcumin Releasing Nanoparticles in Coconut Oil:

The formulation as in the above description except that the nanoparticles are uniformly mixed into powdered coconut oil (proprietary process) and compacted into a suitable block or roll on configuration for topical application. The use of the melted coconut oil (in the above formulation) has limitations (although still feasible) because there is some release of curcumin from the nanoparticles once they are mixed into liquid coconut oil. In contrast there is no release when the nanoparticles are mixed with the powdered form of the coconut oil.

Additional variations include the use of colorless curcumin or chemically modified curcumin. Other variations can include the use of other oils or mixtures with other oils such as butter of cacoa mixed with coconut oil to improve the consistency and melting temperature of the solid formulation.

Efficacy:

Curcumin containing coconut oil was applied to the following body parts at an amount that created a permanent (˜2 to 3 weeks) yellow stain at the site of administration: 1) knee (arthritic (osteoarthritis) and inflamed); 2) back; 3) thigh; and 4) face.

Effect on Blood Pressure:

Application of several mls of the material to any the sites other than the face (limitations as to the amount that can be applied due to the yellow staining effects), produced a very noticeable drop in both systolic and diastolic blood pressure (typical values: ˜120-100 mm Hg for systolic, and ˜84-70 mm Hg for diastolic). The initial drop lasted approximately one hour after which the values increased slightly but remained in the regime of 110/73 mm Hg for at least three weeks. This testing was done primarily on one test subject (5 applications spaced out over a period of months) but similar results were obtained on a second test subject (two applications separated by several months). No adverse effects were noticed except for an initial short period (˜15 minutes) of light headedness when large amounts were applied).

Effect on Inflamed Arthritic Knee:

Subjective sustained improvement in mobility with a concomitant reduction in pain. Effect appears to persist for two to three weeks following each application.

### 6.20 Efficacy of Curcumin Nanoparticles on Osteoarthritis (OA)

In this example, mice with OA (destabilization of the medial meniscus, DMM model, 8 weeks) were treated daily, starting on the day of OA surgery with topical nano-encapsulated curcumin (nano-curcumin, 7 mg nanoparticles, 70 μg curcumin), or with vehicle (coconut oil) alone.

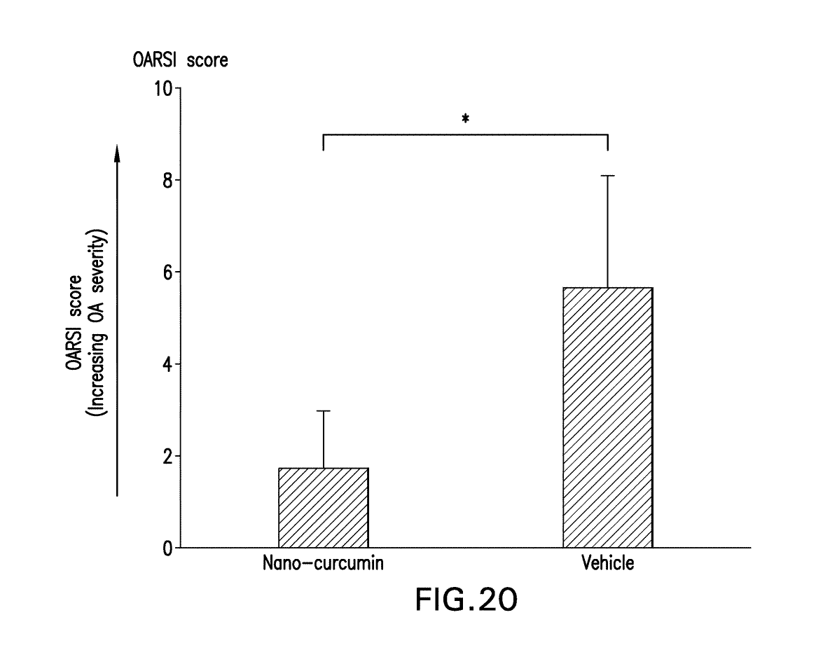

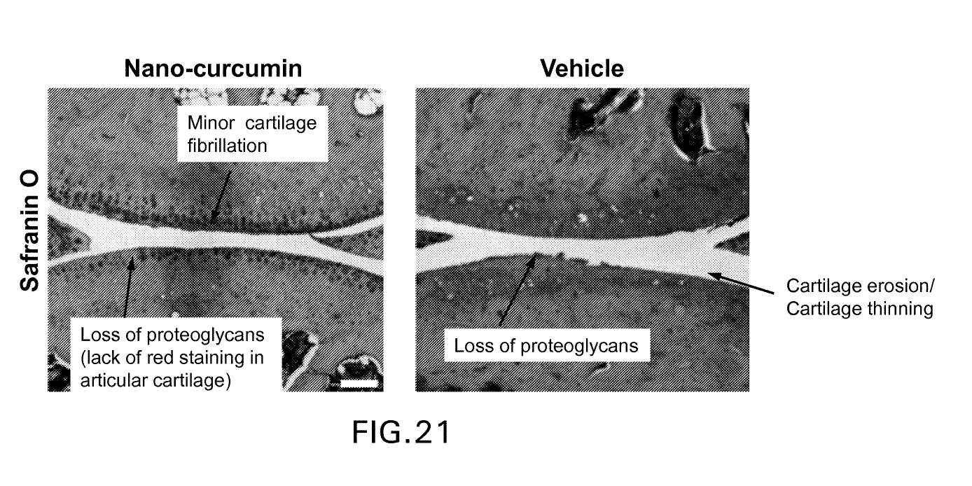

The results showed that nano-curcumin-treated mice exhibited a lower OA histologic score (using the OARSI scoring system) compared to OA mice treated with vehicle (FIG. 20; *p<0.05. n=3/group). Safranin O staining revealed OA mice treated with nano-encapsulated curcumin had cartilage with minor superficial damage, and loss of proteoglycans, while vehicle-treated mice exhibited cartilage erosion and a severe loss of proteoglycans (FIG. 21).

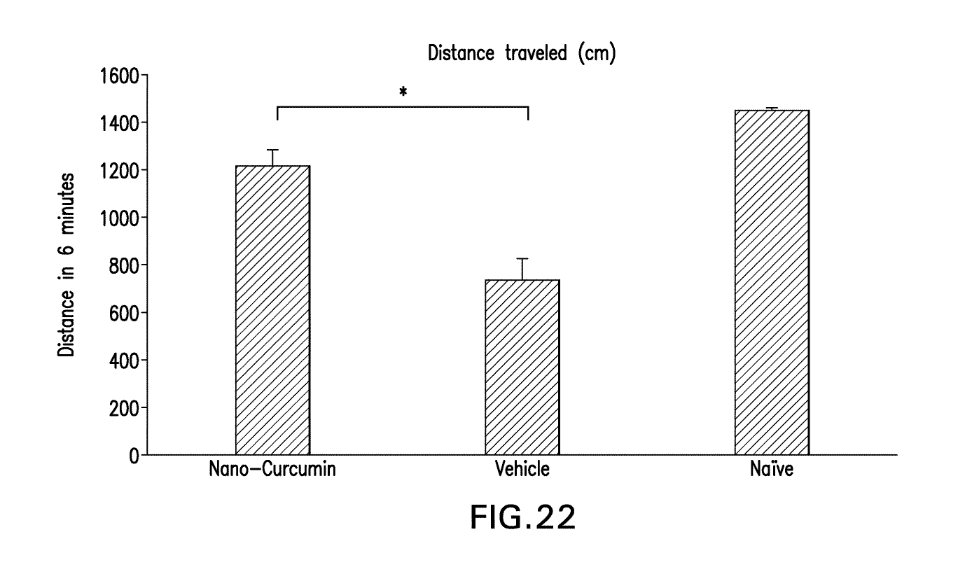

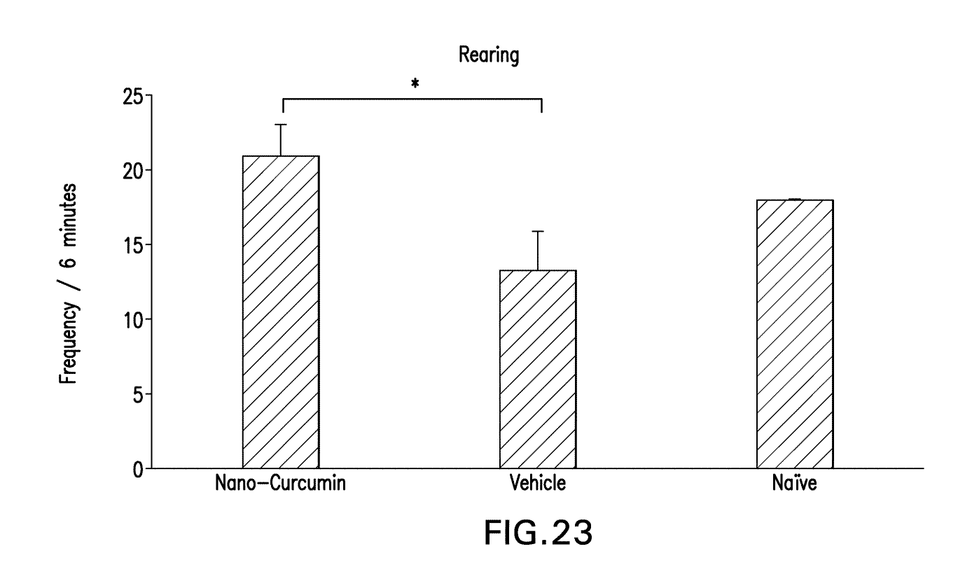

Further, the nano-curcumin-treated mice traveled a longer distance (FIG. 22), and reared more often (stood on their hind limbs) in an open box assay (FIG. 23), compared to vehicle-treated mice, and exhibited locomotive behaviors similar to naïve mice. *p<0.05. n=3/group.

The following examples (6.21-6.22) refer to the efficacy of myristic acid encapsulated nanoparticles in accordance with one or more embodiments of the present application.

### 6.21 Efficacy of Myristic Acid Encapsulated Nanoparticles in Treating Erectile Dysfunction (ED)

In this example, several healthy male rats underwent surgery to transect the cavernous nerve. The following experiments were conducted two months after the surgery. At that point, there were two factors operating to inhibit erectile activity: i) the transected cavernous nerve and ii) the extended period during which the erectile machinery undergoes physical and physiological changes secondary to the absence of stimulation thus making the requisite tissues more recalcitrant with respect to a positive response to any potential external stimulation.

The intracorporal pressure in the penis of these treated rats were monitored as a function of time subsequent to the administration of equivalent amounts of two different NO releasing nanoparticle formulations. Both formulations utilized a myristic acid encapsultated nitric oxide releasing nanoparticle formulation, but in one case the nanoparticles were prepared with a small PEG (400) where the second case utilized a larger PEG (1000). The inclusion of a larger PEG results in a more rapid release of NO.

The slow NO release myristic acid nanoparticles (PEG 400) produced minimal erectile activity but did induce a noticeable drop in systemic blood pressure. The rapid NO release myristic acid nanoparticles (PEG 1000), however, were effective in inducing significant erectile activity. The results were obtained for two rats in each category (four rats total). The results are consistent with the slow release NOnp not able to achieve a threshold level of NO to induce an erection. Furthermore the systemic effect of lowered blood pressure also works against achieving an erection. The more rapid release platform can create local concentrations that exceed the needed threshold in these extreme models of erectile dysfunction.

### 6.22 Efficacy of Myristic Acid Encapsulated Nanoparticles for Cardiovascular Endpoints

In this example, 5 mg of NO-releasing nanoparticles with and without myristic acid were applied into the cheek pouch of hamsters. More specifically, the hamsters were put into three treatment groups: 1) NO-nanoparticles with myristic acid (n=3) [NO-np-C14H28O2]; 2) NO-no without myristic acid (n=3) [NO-np]; and untreated (n=5).

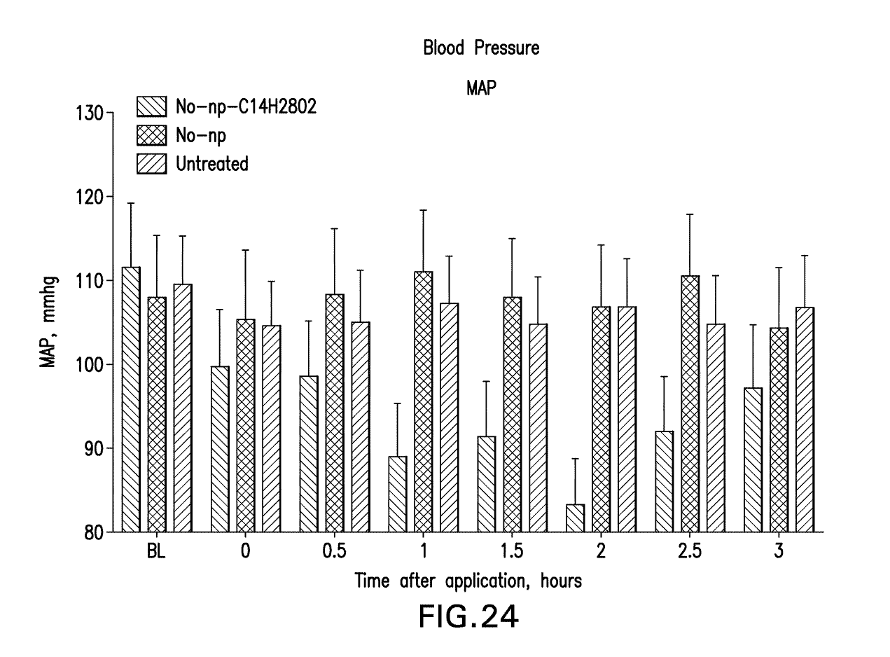

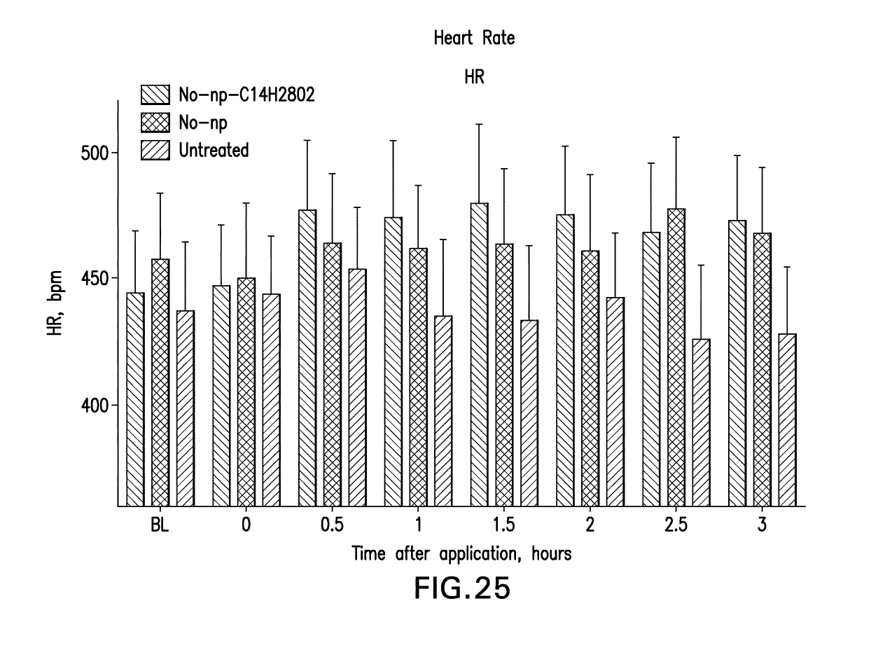

The results showed that treatment with NO-np-C14H28O2 resulted in greater decreases in blood pressure (mean artery pressure [MAP]) relative to baseline at all time points compared with treatment with NO-np and untreated (FIG. 24). Further, treatment with NO-np-C14H28O2 also resulted in greater increases in heart rate (beats per minute [bpm]) relative to baseline at all time points compared with treatment with NO-np and untreated (FIG. 25).

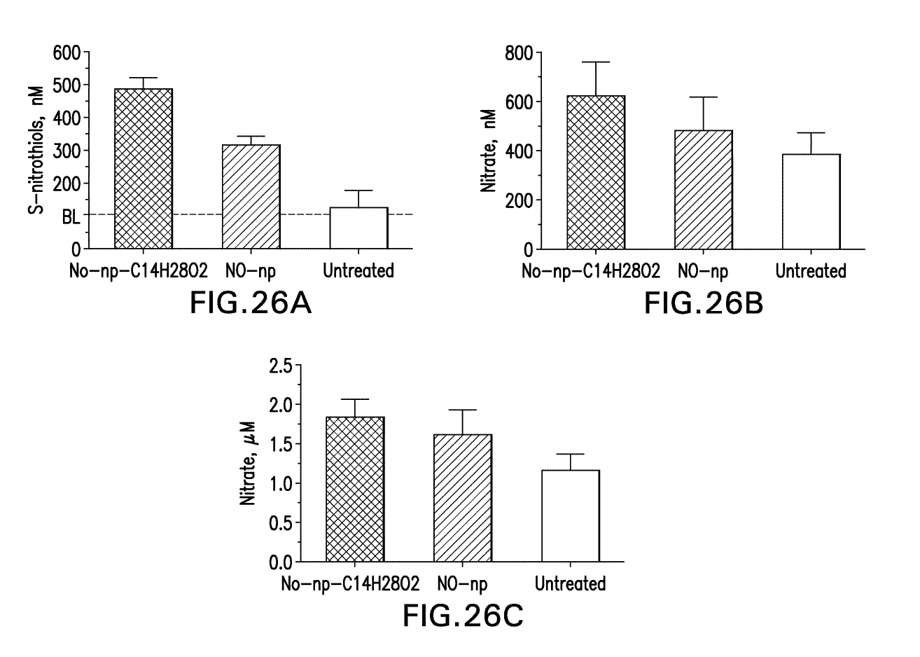

FIG. 26 shows the levels of NO-related products (S-nitrosothiols [FIG. 26A], nitrite [FIG. 26B], and nitrate [FIG. 26C]) in the blood following treatment for each treatment group. S-nitrosothiols, nitrite, and nitrate are all by products of NO being released into the circulation (either directly via slow release of circulating nanoparticles that have entered the bloodstream or a trickling into the bloodstream of NO and its byproducts from the local site where the nanoparticles were administered). The results show that treatment with NO-np-C14H28O2 resulted in greater levels of NO-related products entering the blood as compared with treatment with NO-np and untreated. These results suggest that NO-np-C14H28O2 penetrate more effectively, get into the blood stream more effectively, and/or create more NO-related products that get into the circulation. The time dependent change in blood pressure (FIG. 24) is also consistent with greater delivery of NO and NO-related products into the circulation with the NO-np-C14H28O2.

The present application is not to be limited in scope by the specific embodiments described herein. Indeed, various modifications of embodiments of the present application in addition to those described will become apparent to those skilled in the art from the foregoing description and accompanying figures. Such modifications are intended to fall within the scope of the appended claims. All references cited below are incorporated herein by reference in their entirety and for all purposes to the same extent as if each individual publication or patent or patent application was specifically and individually indicated to be incorporated by reference in its entirety for all purposes.

### REFERENCES

1. Adimi P, Hashemi S J, Mahmoudi M et al. In-vitro Activity of 10 Antifungal Agents against 320 Dermatophyte Strains Using Microdilution Method in Tehran. Iranian journal of pharmaceutical research: IJPR 2013; 12: 537-45.
2. Ahn J C, Kang J W, Shin J I et al. Combination treatment with photodynamic therapy and curcumin induces mitochondria-dependent apoptosis in AMC-HN3 cells. International journal of oncology 2012; 41: 2184-90.
3. Ahren M, Selegard L, Klasson A, Soderlind F, Abrikossova N, Skoglund C, Bengtsson T, Engstrom M, Kall P O, Uvdal K: Synthesis and characterization of PEGylated Gd2O3 nanoparticles for MRI contrast enhancement, Langmuir 2010, 26:5753-5762.
4. Altman S A, Randers L, Rao G. Comparison of trypan blue dye exclusion and fluorometric assays for mammalian cell viability determinations. Biotechnol Prog. 1993; 9(6):671-674.
5. Anand P, Sundaram C, Jhurani S, et al. Curcumin and cancer: an “old-age” disease with an “age-old” solution. Cancer Lett. 2008; 267(1):133-164.
6. Anitha A, Deepagan V, Divya Rani V, et al. Preparation, characterization, in vitro drug release and biological studies of curcumin loaded dextran sulphate-chitosan nanoparticles. Carbohydrate Polymers. 2011; 84(3):1158-1164.
7. Apisariyakul A, Vanittanakom N, Buddhasukh D. Antifungal activity of turmeric oil extracted from Curcuma longa (Zingiberaceae). Journal of ethnopharmacology 1995; 49: 163-9.
8. Araujo J, Logothetis C: Dasatinib: a potent SRC inhibitor in clinical development for the treatment of solid tumors, Cancer Treat Rev 2010, 36:492-500.
9. Araujo N C, Fontana C R, Bagnato V S et al. Photodynamic antimicrobial therapy of curcumin in biofilms and carious dentine. Lasers in medical science 2013.
10. Arunraj T, Rejinold N S, Mangalathillam S, et al. Synthesis, Characterization and Biological Activities of Curcumin Nanospheres. Journal of Biomedical Nanotechnology. 2014; 10(2):238-250.
11. Auletta C S: Acute, subchronic, and chronic toxicology. Edited by Derelanko M J, Hollinger M A. Boca Raton, London, New York, Washington D.C., CRC Press, 2002, pp. 69-126.
12. Aziz Z, Abu S F, Chong N J. A systematic review of silver-containing dressings and topical silver agents (used with dressings) for burn wounds. Burns. 2012; 38(3):307-318.
13. Baltazar Lde M, Santos P C, Paula T P et al. IFN-gamma impairs Trichophyton rubrum proliferation in a murine model of dermatophytosis through the production of IL-1beta and reactive oxygen species. Medical mycology 2014; 52: 293-302.
14. Baltazar Lde M, Soares B M, Carneiro H C et al. Photodynamic inhibition of Trichophyton rubrum: in vitro activity and the role of oxidative and nitrosative bursts in fungal death. The Journal of antimicrobial chemotherapy 2013; 68: 354-61.
15. Bhawana, Basniwal R K, Buttar H S, et al. Curcumin nanoparticles: preparation, characterization, and antimicrobial study. J Agric Food Chem. 2011; 59(5):2056-2061.
16. Bogdan C. Nitric oxide and the immune response. Nature immunology. October 2001; 2(10):907-916.
17. Broughton 2nd G, Janis J E, Attinger C E. The basic science of wound healing. Plast Reconstr Surg. 2006; 117(7 Suppl):12S-34S.
18. Bruzell E M, Morisbak E, Tonnesen H H. Studies on curcumin and curcuminoids. XXIX. Photoinduced cytotoxicity of curcumin in selected aqueous preparations. Photochemical & photobiological sciences: Official journal of the European Photochemistry Association and the European Society for Photobiology 2005; 4: 523-30.
19. Buytaert E, Dewaele M, Agostinis P. Molecular effectors of multiple cell death pathways initiated by photodynamic therapy. Biochimica et biophysica acta 2007; 1776: 86-107.
20. Calzavara-Pinton P, Rossi M T, Sala R et al. Photodynamic antifungal chemotherapy. Photochemistry and photobiology 2012; 88: 512-22.
21. Cheppudira B, Fowler M, McGhee L, et al. Curcumin: a novel therapeutic for burn pain and wound healing. Expert Opin Investig Drugs. 2013; 22(10):1295-1303.
22. Chereddy K K, Coco R, Memvanga P B, et al. Combined effect of PLGA and curcumin on wound healing activity. J Control Release. 2013; 171(2):208-215.
23. Chirgwin J M, Guise T A: Skeletal metastases: decreasing tumor burden by targeting the bone microenvironment, J Cell Biochem 2007, 102:1333-1342
24. Chouake J, Schairer D, Kutner A, et al. Nitrosoglutathione generating nitric oxide nanoparticles as an improved strategy for combating Pseudomonas aeruginosa-infected wounds. Journal of drugs in dermatology: JDD. December 2012; 11(12):1471-1477.
25. Church D, Elsayed S, Reid O, et al. Burn wound infections. Clin Microbiol Rev. 2006; 19(2):403-434.
26. Coelho L M, Aquino-Ferreira R, Maffei C M et al. In vitro antifungal drug susceptibilities of dermatophytes microconidia and arthroconidia. The Journal of antimicrobial chemotherapy 2008; 62: 758-61.
27. Conway J G, McDonald B, Parham J, Keith B, Rusnak D W, Shaw E, Jansen M, Lin P, Payne A, Crosby R M, Johnson J H, Frick L, Lin M H, Depee S, Tadepalli S, Votta B, James I, Fuller K, Chambers T J, Kull F C, Chamberlain S D, Hutchins J T: Inhibition of colony-stimulating-factor-1 signaling in vivo with the orally bioavailable cFMS kinase inhibitor GW2580, Proc Natl Acad Sci USA 2005, 102:16078-16083.
28. Cooke J P, Andon N, Loscalzo J. S-nitrosocaptopril. II. Effects on vascular reactivity. The Journal of pharmacology and experimental therapeutics. June 1989; 249(3):730-734.
29. Dahl T A, McGowan W M, Shand M A et al. Photokilling of bacteria by the natural dye curcumin. Archives of microbiology 1989; 151: 183-5.
30. Dai T, Fuchs B B, Coleman J J et al. Concepts and principles of photodynamic therapy as an alternative antifungal discovery platform. Frontiers in microbiology 2012; 3: 120.
31. De Groote M A, Fang F C. NO inhibitions: antimicrobial properties of nitric oxide. Clin Infect Dis. 1995; 21 Suppl 2:S162-165.
32. De Groote M A, Fang F C. NO inhibitions: antimicrobial properties of nitric oxide. Clinical infectious diseases: an official publication of the Infectious Diseases Society of America 1995; 21 Suppl 2: S162-5.
33. de Macedo J L, Santos J B. Bacterial and fungal colonization of burn wounds. Mem Inst Oswaldo Cruz. 2005; 100(5):535-539.
34. Diefenbach A, Schindler H, Rollinghoff M, Yokoyama W M, Bogdan C. Requirement for type 2 NO synthase for IL-12 signaling in innate immunity. Science. May 7 1999; 284(5416):951-955.
35. Donnelly R F, McCarron P A, Tunney M M. Antifungal photodynamic therapy. Microbiological research 2008; 163: 1-12.
36. Dovigo L N, Carmello J C, de Souza Costa C A et al. Curcumin-mediated photodynamic inactivation of Candida albicans in a murine model of oral candidiasis. Medical mycology 2013; 51: 243-51.
37. Dovigo L N, Pavarina A C, Ribeiro A P et al. Investigation of the photodynamic effects of curcumin against Candida albicans. Photochemistry and photobiology 2011; 87: 895-903.
38. Ecological Effects Test Guidelines: OPPTS 850.1075 Fish Acute Toxicity Test, Freshwater and Marine. United States Environmental Protection Agency; 1996.
39. Fang F C. Perspectives series: host/pathogen interactions. Mechanisms of nitric oxide-related antimicrobial activity. The Journal of clinical investigation 1997; 99: 2818-25.
40. Flatley J, Barrett J, Pullan S T, Hughes M N, Green J, Poole R K. Transcriptional responses of Escherichia coli to S-nitrosoglutathione under defined chemostat conditions reveal major changes in methionine biosynthesis. The Journal of biological chemistry. Mar. 18 2005; 280(11):10065-10072.
41. Flora G, Gupta D, Tiwari A. Nanocurcumin: a promising therapeutic advancement over native curcumin. Crit Rev Ther Drug Carrier Syst. 2013; 30(4):331-368.
42. Friedman A, Blecher K, Sanchez D, et al. Susceptibility of Gram-positive and − negative bacteria to novel nitric oxide-releasing nanoparticle technology. Virulence. May-June 2011; 2(3):217-221.
43. Friedman A J, Blecher K, Schairer D, et al. Improved antimicrobial efficacy with nitric oxide releasing nanoparticle generated S-nitrosoglutathione. Nitric oxide: biology and chemistry/official journal of the Nitric Oxide Society. Nov. 30 2011; 25(4):381-386.
44. Friedman A J, Han G, Navati M S et al. Sustained release nitric oxide releasing nanoparticles: characterization of a novel delivery platform based on nitrite containing hydrogel/glass composites. Nitric oxide: biology and chemistry/official journal of the Nitric Oxide Society 2008; 19: 12-20.
45. Gaston B. Nitric oxide and thiol groups. Biochimica et biophysica acta. May 5 1999; 1411(2-3):323-333.
46. Ghelardi E, Celandroni F, Gueye S A et al. Potential of Ergosterol synthesis inhibitors to cause resistance or cross-resistance in Trichophyton rubrum. Antimicrobial agents and chemotherapy 2014; 58: 2825-9.
47. Godley B F, Shamsi F A, Liang F Q et al. Blue light induces mitochondrial DNA damage and free radical production in epithelial cells. The Journal of biological chemistry 2005; 280: 21061-6.
48. Gopinath D, Ahmed M R, Gomathi K, et al. Dermal wound healing processes with curcumin incorporated collagen films. Biomaterials. 2004; 25(10):1911-1917.
49. Guimaraes A J, Frases S, Gomez F J et al. Monoclonal antibodies to heat shock protein 60 alter the pathogenesis of Histoplasma capsulatum. Infection and immunity 2009; 77: 1357-67.
50. Gunes H, Gulen D, Mutlu R, et al. Antibacterial effects of curcumin: an in vitro minimum inhibitory concentration study. Toxicol Ind Health. 2013.
51. Gupta A K, Humke S. The prevalence and management of onychomycosis in diabetic patients. European journal of dermatology: ETD 2000; 10: 379-84.
52. Gupta R, Kumar A. Bioactive materials for biomedical applications using sol-gel technology. Biomedical Materials. 2008; 3(3):034005.
53. Gupta S C, Kismali G, Aggarwal B B. Curcumin, a component of turmeric: from farm to pharmacy. Biofactors. 2013; 39(1):2-13.
54. Gupta S C, Patchva S, Aggarwal B B. Therapeutic roles of curcumin: lessons learned from clinical trials. The AAPS journal. 2013; 15(1):195-218.
55. Hamblin M R, Hasan T. Photodynamic therapy: a new antimicrobial approach to infectious disease? Photochemical & photobiological sciences: Official journal of the European Photochemistry Association and the European Society for Photobiology 2004; 3: 436-50.
56. Han G, Martinez L R, Mihu M R, et al. Nitric oxide releasing nanoparticles are therapeutic for Staphylococcus aureus abscesses in a murine model of infection. PLoS One. 2009; 4(11):e7804.
57. Han G, Tar M, Kuppam D S, et al. Nanoparticles as a novel delivery vehicle for therapeutics targeting erectile dysfunction. J Sex Med. 2010; 7(1 Pt 1):224-233.
58. Haukvik T, Bruzell E, Kristensen S et al. Photokilling of bacteria by curcumin in different aqueous preparations. Studies on curcumin and curcuminoids XXXVII. Die Pharmazie 2009; 64: 666-73.
59. Hegge A B, Andersen T, Melvik J E et al. Formulation and bacterial phototoxicity of curcumin loaded alginate foams for wound treatment applications: studies on curcumin and curcuminoides XLII. Journal of pharmaceutical sciences 2011; 100: 174-85.
60. Hegge A B, Bruzell E, Kristensen S et al. Photoinactivation of Staphylococcus epidermidis biofilms and suspensions by the hydrophobic photosensitizer curcumin—effect of selected nanocarrier: studies on curcumin and curcuminoides XLVII. European journal of pharmaceutical sciences: official journal of the European Federation for Pharmaceutical Sciences 2012; 47: 65-74.
61. Hegge A B, Nielsen T T, Larsen K L et al Impact of curcumin supersaturation in antibacterial photodynamic therapy—effect of cyclodextrin type and amount: studies on curcumin and curcuminoides XLV. Journal of pharmaceutical sciences 2012; 101: 1524-37.
62. Hegge A B, Vukicevic M, Bruzell E et al. Solid dispersions for preparation of phototoxic supersaturated solutions for antimicrobial photodynamic therapy (aPDT): Studies on curcumin and curcuminoides L. European journal of pharmaceutics and biopharmaceutics: official journal of Arbeitsgemeinschaft fur Pharmazeutische Verfahrenstechnik eV 2013; 83: 95-105.
63. Henry P J, Drummer O H, Horowitz J D. S-nitrosothiols as vasodilators: implications regarding tolerance to nitric oxide-containing vasodilators. British journal of pharmacology. November 1989; 98(3):757-766.
64. Institute CaLS. Reference method for broth dilution antifungal susceptibility testing of filamentous fungi. CLSI, Wayne, Pa., 2008.
65. Irmler I, Schmidt K, Starck J M. Developmental variability during early embryonic development of zebra fish, Danio rerio. Journal of experimental zoology Part B, Molecular and developmental evolution. 2004; 302(5):446-457.
66. Irvine K M, Burns C J, Wilks A F, Su S, Hume D A, Sweet M J: A CSF-1 receptor kinase inhibitor targets effector functions and inhibits pro-inflammatory cytokine production from murine macrophage populations, Faseb J 2006, 20:1921-1923.
67. Jarboe L R, Hyduke D R, Tran L M, Chou K J, Liao J C. Determination of the Escherichia coli S-nitrosoglutathione response network using integrated biochemical and systems analysis. The Journal of biological chemistry. Feb. 22 2008; 283(8):5148-5157.
68. Jori G, Fabris C, Soncin M et al. Photodynamic therapy in the treatment of microbial infections: basic principles and perspective applications. Lasers in surgery and medicine 2006; 38: 468-81.
69. Kleinpenning M M, Smits T, Frunt M H et al. Clinical and histological effects of blue light on normal skin. Photodermatology, photoimmunology & photomedicine 2010; 26: 16-21.
70. Kowaluk E A, Poliszczuk R, Fung H L. Tolerance to relaxation in rat aorta: comparison of an S-nitrosothiol with nitroglycerin. European journal of pharmacology. Dec. 15 1987; 144(3):379-383.
71. Kozlow W, Guise T A: Breast cancer metastasis to bone: mechanisms of osteolysis and implications for therapy, J Mammary Gland Biol Neoplasia 2005, 10:169-180.
72. Kulac M, Aktas C, Tulubas F, et al. The effects of topical treatment with curcumin on burn wound healing in rats. Journal of molecular histology. 2013; 44(1):83-90.
73. Kumar S S D, Surianarayanan M, Vijayaraghavan R, et al. Curcumin loaded poly (2-hydroxyethyl methacrylate) nanoparticles from gelled ionic liquid—In vitro cytotoxicity and anti-cancer activity in SKOV-3 cells. Eur J Pharm Sci. 2014; 51:34-44.
74. Kunwar A, Batik A, Mishra B, et al. Quantitative cellular uptake, localization and cytotoxicity of curcumin in normal and tumor cells. Biochimica et Biophysica Acta (BBA)—General Subjects. 2008; 1780(4):673-679.
75. Lee C, Miura K, Liu X, Zweier J L. Biphasic regulation of leukocyte superoxide generation by nitric oxide and peroxynitrite. The Journal of biological chemistry. Dec. 15 2000; 275(50):38965-38972.
76. Li Q, Frank M, Thisse C I, et al. Zebrafish: a model system to study heritable skin diseases. J Invest Dermatol. 2011; 131(3):565-571.
77. Liebmann J, Born M, Kolb-Bachofen V. Blue-light irradiation regulates proliferation and differentiation in human skin cells. The Journal of investigative dermatology 2010; 130: 259-69.
78. Liew F Y, Cox F E. Nonspecific defence mechanism: the role of nitric oxide. Immunology today. March 1991; 12(3):A17-21.
79. Lin E Y, Gouon-Evans V, Nguyen A V, Pollard J W: The macrophage growth factor, CSF-1, in mammary gland development and cancer, Journal of Mammary Gland Development and Neoplasia 2002, 7:147-162
80. Lin E Y, Li J-F, Bricard G, Wang W, Deng Y, Sellers R, Porcelli S A, Pollard J W: VEGF restores delayed tumor progression in tumors depleted of macrophages, Molecular Oncology, 2007, 1:288.
81. Lin E Y, Li J F, Gnatovskiy L, Deng Y, Zhu L, Grzesik D A, Qian H, Xue X N, Pollard J W: Macrophages regulate the angiogenic switch in a mouse model of breast cancer, Cancer Res 2006, 66:11238-11246.
82. Lin E Y, Nguyen A V, Russell R G, Pollard J W: Colony-stimulating factor 1 promotes progression of mammary tumors to malignancy, J Exp Med 2001, 193:727-740.
83. Lin E Y, Orlofsky A, Berger M S, Prystowsky M B: Characterization of A1, a novel hemopoietic-specific early-response gene with sequence similarity to bcl-2, J Immunol 1993, 151:1979-1988.
84. Lin E Y, Orlofsky A, Wang H G, Reed J C, Prystowsky M B: A1, a Bcl-2 family member, prolongs cell survival and permits myeloid differentiation, Blood 1996, 87:983-992.
85. Lin E Y, Pollard J W: Tumor-associated macrophages press the angiogenic switch in breast cancer, Cancer Res 2007, 67:5064-5066.
86. Loscalzo J, Smick D, Andon N, Cooke J. S-nitrosocaptopril. I. Molecular characterization and effects on the vasculature and on platelets. The Journal of pharmacology and experimental therapeutics. June 1989; 249(3):726-729.
87. Mani H, Sidhu G S, Kumari R, et al. Curcumin differentially regulates TGF-beta1, its receptors and nitric oxide synthase during impaired wound healing. Biofactors. 2002; 16(1-2):29-43.
88. Manzano-Gayosso P, Mendez-Tovar L J, Hernandez-Hernandez F et al. [Antifungal resistance: an emerging problem in Mexico]. Gaceta medica de Mexico 2008; 144: 23-6.
89. Martinez L R, Han G, Chacko M, et al. Antimicrobial and healing efficacy of sustained release nitric oxide nanoparticles against Staphylococcus aureus skin infection. J Invest Dermatol. 2009; 129(10):2463-2469.
90. Martinez-Rossi N M, Peres N T, Rossi A. Antifungal resistance mechanisms in dermatophytes. Mycopathologia 2008; 166: 369-83.
91. Mizutani K, Sud S, McGregor N A, Martinovski G, Rice B T, Craig M J, Varsos Z S, Roca H, Pienta K J: The chemokine CCL2 increases prostate tumor growth and bone metastasis through macrophage and osteoclast recruitment, Neoplasia 2009, 11:1235-1242.
92. Mohanty C, Das M, Sahoo S K. Sustained wound healing activity of curcumin loaded oleic acid based polymeric bandage in a rat model. Mol Pharm. 2012; 9(10):2801-2811.
93. Morgan G, Lipton A: Antitumor effects and anticancer applications of bisphosphonates, Semin Oncol 2010, 37 Suppl 2:S30-40.
94. Nacharaju P, Tuckman-Vernon C, Maier K E, et al. A nanoparticle delivery vehicle for S-nitroso-N-acetyl cysteine: sustained vascular response. Nitric oxide: biology and chemistry/official journal of the Nitric Oxide Society. Oct. 15 2012; 27(3):150-160.
95. Nanomaterial-Biological Interactions Knowledgebase. ONAMI & Oregon State University; 2010. http://nbi.oregonstate.edu/.
96. Narayanan A, Kehn-Hall K, Senina S et al. Curcumin inhibits Rift Valley fever virus replication in human cells. The Journal of biological chemistry 2012; 287: 33198-214.
97. Nguyen T A, Friedman A J. Curcumin: a novel treatment for skin-related disorders. J Drugs Dermatol. 2013; 12(10):1131-1137.
98. Park B S, Kim J G, Kim M R, et al. Curcuma longa L. constituents inhibit sortase A and Staphylococcus aureus cell adhesion to fibronectin. J Agric Food Chem. 2005; 53(23):9005-9009.
99. Park S I, Sold F N, McCauley L K: Roles of Bone Marrow Cells in Skeletal Metastases: No Longer Bystanders, Cancer Microenviron 2011.
100. Pavlakis N, Schmidt R, Stockier M: Bisphosphonates for breast cancer, Cochrane Database Syst Rev 2005, CD003474.
101. Pawloski J R, Hess D T, Stamler J S. Export by red blood cells of nitric oxide bioactivity. Nature. Feb. 1 2001; 409(6820):622-626.
102. Pelgrift R Y, Friedman A J. Nanotechnology as a therapeutic tool to combat microbial resistance. Advanced drug delivery reviews. Nov. 30 2013; 65(13-14):1803-1815.
103. Perri S, Prokopovich P, Pratten J et al. Nanoparticles: their potential use in antibacterial photodynamic therapy. Photochemical & photobiological sciences: Official journal of the European Photochemistry Association and the European Society for Photobiology 2011; 10: 712-20.
104. Priyadarsini K. Photophysics, photochemistry and photobiology of curcumin: Studies from organic solutions, bio-mimetics and living cells. Journal of Photochemistry and Photobiology C:Photochemistry Reviews 2009; 10: 81-95.
105. Pullan S T, Gidley M D, Jones R A, et al. Nitric oxide in chemostat-cultured Escherichia coli is sensed by Fnr and other global regulators: unaltered methionine biosynthesis indicates lack of S nitrosation. Journal of bacteriology. March 2007; 189(5):1845-1855.
106. Radi R, Cassina A, Hodara R. Nitric oxide and peroxynitrite interactions with mitochondria. Biological chemistry 2002; 383: 401-9.
107. Rai D, Singh J K, Roy N, et al. Curcumin inhibits FtsZ assembly: an attractive mechanism for its antibacterial activity. Biochem J. 2008; 410(1):147-155.
108. Richardson A R, Dunman P M, Fang F C. The nitrosative stress response of Staphylococcus aureus is required for resistance to innate immunity. Molecular microbiology. August 2006; 61(4):927-939.

109. Richardson A R, Libby S J, Fang F C. A nitric oxide-inducible lactate dehydrogenase enables Staphylococcus aureus to resist innate immunity. Science. Mar. 21 2008; 319(5870):1672-1676.
110. Richardson G, Benjamin N. Potential therapeutic uses for S-nitrosothiols. Clinical science. January 2002; 102(1):99-105.
111. Rizzo L Y, Golombek S K, Mertens M E, et al. In vivo nanotoxicity testing using the zebrafish embryo assay. Journal of Materials Chemistry B. 2013; 1(32):3918-3925.
112. Roodman G D: Mechanisms of bone metastasis, N Engl J Med 2004, 350:1655-1664.
113. Rotta I, Sanchez A, Goncalves P R et al. Efficacy and safety of topical antifungals in the treatment of dermatomycosis: a systematic review. The British journal of dermatology 2012; 166: 927-33.
114. Rudrappa T, Bais H P. Curcumin, a known phenolic from Curcuma longa, attenuates the virulence of Pseudomonas aeruginosa PAO1 in whole plant and animal pathogenicity models. J Agric Food Chem. 2008; 56(6):1955-1962.
115. Ruggiero S L, Mehrotra B, Rosenberg T J, Engroff S L: Osteonecrosis of the jaws associated with the use of bisphosphonates: a review of 63 cases, J Oral Maxillofac Surg 2004, 62:527-534.
116. Sanchez D A, Schairer D, Tuckman-Vernon C, et al. Amphotericin B releasing nanoparticle topical treatment of Candida spp. in the setting of a burn wound. Nanomedicine. 2013.
117. Sandur S K, Ichikawa H, Pandey M K, et al. Role of pro-oxidants and antioxidants in the anti-inflammatory and apoptotic effects of curcumin (diferuloylmethane). Free Radic Biol Med. 2007; 43(4):568-580.
118. Sanoj Rejinold N, Sreerekha P, Chennazhi K, et al. Biocompatible, biodegradable and thermo-sensitive chitosan-g-poly (N-isopropylacrylamide) nanocarrier for curcumin drug delivery. Int J Biol Macromol. 2011; 49(2):161-172.
119. Santos D A, Banos M E, Hamdan J S. Establishing a method of inoculum preparation for susceptibility testing of Trichophyton rubrum and Trichophyton mentagrophytes. Journal of clinical microbiology 2006; 44: 98-101.
120. Santos D A, Hamdan J S. In vitro antifungal oral drug and drug-combination activity against onychomycosis causative dermatophytes. Medical mycology 2006; 44: 357-62.
121. Schairer D O, Chouake J S, Nosanchuk J D et al. The potential of nitric oxide releasing therapies as antimicrobial agents. Virulence 2012; 3: 271-9.
122. Schmid M C, Varner J A: Myeloid cells in the tumor microenvironment: modulation of tumor angiogenesis and tumor inflammation, J Oncol 2010, 2010:201026.
123. Seleem M N, Munusamy P, Ranjan A, et al. Silica-antibiotic hybrid nanoparticles for targeting intracellular pathogens. Antimicrob Agents Chemother. 2009; 53(10):4270-4274.
124. Shaffer J E, Lee F, Thomson S, Han B J, Cooke J P, Loscalzo J. The hemodynamic effects of S-nitrosocaptopril in anesthetized dogs. The Journal of pharmacology and experimental therapeutics. February 1991; 256(2):704-709.
125. Sharma M, Manoharlal R, Puri N et al. Antifungal curcumin induces reactive oxygen species and triggers an early apoptosis but prevents hyphae development by targeting the global repressor TUP1 in Candida albicans. Bioscience reports 2010; 30: 391-404.
126. Shehzad A, Lee Y S. Molecular mechanisms of curcumin action: signal transduction. Biofactors. 2013; 39(1):27-36.
127. Shen L, H. F. Ji and H. Y. Zhang. A TD-DFT study on triplet excited-state properties of curcumin and its implications in elucidating the photosensitizing mechanisms of the pigment. Chem Phys Lett 2005; 409: 300-3.
128. Sidhu G S, Singh A K, Thaloor D, et al. Enhancement of wound healing by curcumin in animals. Wound Repair Regen. 1998; 6(2):167-177.
129. Singh R, Kristensen S, Tonnesen H H. Influence of cosolvents, ionic strength and the method of sample preparation on the solubilization of curcumin by Pluronics and HP-gamma-cyclodextrin. Studies of curcumin and curcuminoids, XLIV. Die Pharmazie 2012; 67: 131-42.
130. Smijs T G, Pavel S. The susceptibility of dermatophytes to photodynamic treatment with special focus on Trichophyton rubrum. Photochemistry and photobiology 2011; 87: 2-13.
131. Solinas G, Germano G, Mantovani A, Allavena P: Tumor-associated macrophages (TAM) as major players of the cancer-related inflammation, J Leukoc Biol 2009, 86:1065-1073.
132. Somparn P, Phisalaphong C, Nakornchai S, et al. Comparative antioxidant activities of curcumin and its demethoxy and hydrogenated derivatives. Biol Pharm Bull. 2007; 30(1):74-78.
133. Strahle U, Scholz S, Geisler R, et al. Zebrafish embryos as an alternative to animal experiments—a commentary on the definition of the onset of protected life stages in animal welfare regulations. Reprod Toxicol. 2012; 33(2):128-132.
134. Syng-ai C, Kumari A L, Khar A. Effect of curcumin on normal and tumor cells: role of glutathione and bcl-2. Mol Cancer Ther. 2004; 3(9):1101-1108.
135. Tonnesen H H, de Vries H, Karlsen J et al. Studies on curcumin and curcuminoids. IX: Investigation of the photobiological activity of curcumin using bacterial indicator systems. Journal of pharmaceutical sciences 1987; 76: 371-3.
136. Topman G, Lin F H, Gefen A. The natural medications for wound healing—Curcumin, Aloe-Vera and Ginger—do not induce a significant effect on the migration kinematics of cultured fibroblasts. J Biomech. 2013; 46(1):170-174.
137. Truong L, Harper S L, Tanguay R L. Evaluation of Embryotoxicity Using the Zebrafish Model. In: Gautier J-C, ed. Drug Safety Evaluation: Methods and Protocols. Vol 691. New York, N.Y.: Humana Press; 2011:271-279.
138. Truong L, Harper S L, Tanguay R L. Evaluation of embryotoxicity using the zebrafish model. Methods Mol Biol. 2011; 691:271-279.
139. Uda N R, Creus M. Selectivity of Inhibition of N-Succinyl-1,1-Diaminopimelic Acid Desuccinylase in Bacteria: The product of dapE-gene Is Not the Target of 1-Captopril Antimicrobial Activity. Bioinorganic chemistry and applications. 2011; 2011:306465.
140. van Sorge N M, Beasley F C, Gusarov I, et al. Methicillin-resistant Staphylococcus aureus bacterial nitric-oxide synthase affects antibiotic sensitivity and skin abscess development. The Journal of biological chemistry. Mar. 1 2013; 288(9):6417-6426.
141. Vatansever F, de Melo W C, Avci P et al. Antimicrobial strategies centered around reactive oxygen species—bactericidal antibiotics, photodynamic therapy, and beyond. FEMS microbiology reviews 2013; 37: 955-89.
142. Vena G A, Chieco P, Posa F et al. Epidemiology of dermatophytoses: retrospective analysis from 2005 to 2010 and comparison with previous data from 1975. The new microbiologica 2012; 35: 207-13.
143. Verallo-Rowell V M, Dillague K M, Syah-Tjundawan B S. Novel antibacterial and emollient effects of coconut and virgin olive oils in adult atopic dermatitis. Dermatitis: contact, atopic, occupational, drug. 2008; 19(6):308-315.
144. Vermout S, Tabart J, Baldo A et al. Pathogenesis of dermatophytosis. Mycopathologia 2008; 166: 267-75.
145. Vindenes H, Bjerknes R. Microbial colonization of large wounds. Burns. 1995; 21(8):575-579.
146. Wang Y, Lu Z, Wu H, et al. Study on the antibiotic activity of microcapsule curcumin against foodborne pathogens. Int J Food Microbiol. 2009; 136(1):71-74.
147. Wataha J C, Lockwood P E, Lewis J B et al. Biological effects of blue light from dental curing units. Dental materials: official publication of the Academy of Dental Materials 2004; 20: 150-7.
148. Wikene K O, Hegge A B, Bruzell E et al. Formulation and characterization of lyophilized curcumin solid dispersions for antimicrobial photodynamic therapy (aPDT): studies on curcumin and curcuminoids LII. Drug development and industrial pharmacy 2014: 1-9.
149. Woo S B, Hellstein J W, Kalmar J R: Narrative [corrected] review: bisphosphonates and osteonecrosis of the jaws, Ann Intern Med 2006, 144:753-761.
150. Wuthi-udomlert M, Grisanapan W, Luanratana O et al. Antifungal activity of Curcuma longa grown in Thailand. The Southeast Asian journal of tropical medicine and public health 2000; 31 Suppl 1: 178-82.
151. Yuan H, Bao X, Du Y-Z, et al. Preparation and evaluation of SiO₂-deposited stearic acid-g-chitosan nanoparticles for doxorubicin delivery. Int J Nanomedicine. 2012; 7:5119.
152. Zhang J, Lu Y, Pienta K J: Multiple roles of chemokine (C-C motif) ligand 2 in promoting prostate cancer growth, J Natl Cancer Inst 2010, 102:522-528.
153. Zorofchian Moghadamtousi S, Abdul Kadir H, Hassandarvish P et al. A Review on Antibacterial, Antiviral, and Antifungal Activity of Curcumin. BioMed research international 2014; 2014: 186864.

1. A method of preparing a hybrid hydrogel paramagnetic nanoparticle comprising the steps of:
(a) hydrolyzing TMOS;
(b) sonicating the hydrolyzed TMOS to form a TMOS solution;
(c) mixing deionized water with gadolinium chloride hexahydrate, europium chloride hexahydrate, PEG, chitosan, and methanol to form a mixture;
(d) vortexing the mixture;
(e) mixing the TMOS solution, an amine-containing silane, and ammonium hydroxide with the mixture to form a hydrogel mixture;
(f) vortexing the hydrogel mixture to form a hydrogel;
(g) lyophilizing the resulting hydrogel to form a dry material;
(h) ball-malling the dry material to form a powder; and
(i) mixing the resulting powder with an amine-binding PEG.

2. The method of claim 1, wherein step (a) comprises mixing TMOS with deionized water and hydrochloric acid.

3. The method of claim 1, wherein the amine-containing silane is 3-aminopropylmethoxysilane.

4. The method of claim 1, wherein step (c) further comprising mixing a therapeutic agent.

5. The method of claim 4, wherein the therapeutic agent is a chemotherapeutic, a nutraceutical, nitric oxide, a nitrosothiol, an imaging agent, melanin, a plasmid, siRNA, a nitro fatty acid, salts and ions or a combination thereof.

6. The method of claim 1, wherein step (c) further comprises mixing the sonicated mixture with one or more NO-responsive fluorophores.

7. The method of claim 6, wherein the fluorophore is diamino fluorescein.

8. A method of preparing a hybrid hydrogel NO-releasing nanoparticle comprising the steps of:
(a) hydrolyzing TMOS;
(b) sonicating the hydrolyzed TMOS to form a TMOS solution;
(c) mixing an unsaturated fatty acid, with sodium nitrite, a buffer solution, PEG, chitosan, and methanol to form a mixture;
(d) vortexing the mixture;
(e) mixing the TMOS solution and an amine-containing silane with the mixture to form a hydrogel mixture;
(f) vortexing the hydrogel mixture to form a hydrogel;
(g) lyophilizing the resulting hydrogel to form a dry material; and
(h) ball-malling the dry material to form a powder.

9. The method of claim 8, wherein the amine-containing silane is 3-aminopropylmethoxysilane.

10. The method of claim 8, wherein the unsaturated fatty acid is a linoleic acid.

11. The method of claim 10, wherein the linoleic acid is a conjugated linoleic acid.

12. The method of claim 8, wherein the unsaturated fatty acid is oleic acid.

13. The method of claim 8, wherein step (a) comprises mixing TMOS with deionized water and hydrochloric acid.

14. A method of preparing a hybrid hydrogel NO-releasing nanoparticle comprising the steps of:
(a) hydrolyzing TMOS;
(b) sonicating the hydrolyzed TMOS to form a TMOS solution;
(c) mixing methanol with polyvinyl alcohol, a buffer solution, glycerol, chitosan, and sodium nitrite to form a mixture;
(d) vortexing the mixture;
(e) mixing the TMOS solution with the mixture to form a hydrogel;
(f) lyophilizing the resulting hydrogel to form a dry material; and
(g) ball-malling the dry material to form a powder.

15. The method of claim 14, wherein step (a) comprises mixing TMOS with deionized water and hydrochloric acid.

16-47. (canceled)
